(19) United States
(12) Patent Application Publication
(10) Pub. No.: US 2026/0042725 A1
(43) Pub. Date: Feb. 12, 2026

(54) TWO-DIMENSIONAL COVALENT ORGANIC FRAMEWORKS FOR HARVESTING MECHANICAL ENERGY

(71) Applicant: City University of Hong Kong, Kowloon (HK)

(72) Inventors: Qichun ZHANG, Ma On Shan (HK); Qianfeng GU, Fengyang City (CN)

(73) Assignee: City University of Hong Kong, Kowloon (HK)

(21) Appl. No.: 19/239,215

(22) Filed: Jun. 16, 2025

Related U.S. Application Data
(60) Provisional application No. 63/682,027, filed on Aug. 12, 2024.

(51) Int. Cl. C07C 43/225; C07C 211/28; C07C 211/30

(52) U.S. Cl. CPC C07C 43/225; C07C 211/28; C07C 211/30

(57) ABSTRACT

The invention provides structures and synthesis of a class of piezoelectric monomers and COFs with fluorine-substituted side chains tethered on the peripheral position of an aromatic moiety (thus different functional side chains). The multi-fluoro groups on the mentioned piezoelectric COFs provide abundant piezoelectric active sites, which enable the application as the active layer for PENGs. The exemplary embodiments also provide a process for the fabrication of a piezoelectric COF and a process for the fabrication of the piezoelectric nanogenerator utilizing mentioned piezoelectric COF.

### CROSS-REFERENCE TO RELATED APPLICATION

This application claims priority to and the benefit of U.S. Provisional Patent Application No. 63/682,027 filed in the United States Patent and Trademark Office on Aug. 12, 2024, the entire contents of which are incorporated herein by reference.

### FIELD OF INVENTION

This invention relates in general to piezoelectric covalent organic frameworks, and their use in energy harvesting devices.

### BACKGROUND OF INVENTION

Self-powered technologies for flexible and wearable electronics have been attracting substantial attention due to the promising potentials in electronic skin, human health monitoring and physiological activities [25]. In this context, developing materials with self-powering capability that can harvest mechanical energy from human gestures is considered an important solution for wearable and portable electronics [26]. To realize energy generation when mechanical stress is applied, high polarization and non-centrosymmetric characteristics of these materials are critical. Due to the energy generation capability during transduction, materials with piezoelectricity (piezoelectrics) have been depicted as promising candidates. Piezoelectrics with non-centrosymmetric characteristics can generate electrical energy upon mechanical deformation. The deformation of the crystal structure upon external forces in a certain direction brings about polarization with spatially separated opposite electrical charges for piezoelectricity.

Piezoelectricity can arise in crystalline materials that are lacking inversion symmetry including a variety of inorganic materials, organic polymers and perovskites [27-29]. Among those piezoelectric materials, piezoelectric polymers are promising alternatives, where polyvinylidene fluoride (PVDF) with high mechanical strength, thermal stability and piezoelectric properties is of great significance. Particularly, j-phase PVDF with lack of symmetry in the distribution of negatively charged fluorine ions can generate electric dipoles (—CH₂/—CF₂ dipoles) and thereby produce piezoelectric response. In this background, PVDF and its copolymers including poly(vinylidene fluoride-trifluoroethylene) [PVDF-TrFE] have drawn lots of interest. In order to obtain high concentration of β-phase PVDF, additives and harsh conditions including mechanical stretching, annealing or strong electric field were employed for treatment. [30] However, the reliance on toxic constituents, complicated synthesis and poor sustainability raises great concern and limits the further deployment in self-powered systems based on those piezoelectrics. Moreover, the random distribution and lack of long-range alignment of dipole moments within polymer chains severely restrict the evolution of domains and the achievement of permanent polarization during electric poling. Therefore, there is a need for piezoelectric materials with low toxicity, good stability and ordered structure.

The discovery of high-performance organic piezoelectric materials is necessary to the development of self-powering technologies. Organic piezoelectric nanogenerators (PENGs) are attractive in harvesting mechanical energy for various self-powering systems. However, their practical applications are severely restricted by their low output open circuit voltage.

Frameworks with low asymmetry and functional N or O-rich groups can be good candidates for energy harvest. Particularly, high piezoelectric response can be obtained in frameworks with reduced symmetry form. For example, UiO-66 (Hf/Zr)-type MOFs with permanently charged groups (—NH₂, —OH, and —COOH) can induce larger polarity of Hf/Zr—O bonds. Blending these MOFs with polymers also induce interfacial polarization at the conductor-insulator interface (also known as Maxwell-Wangner-Sillar polarization) to greatly enhance the piezoelectric constant due to the increased degree of crystallinity and polar β phase content. Compared with these MOFs, two-dimensional (2D) covalent organic frameworks (COFs), renowned for their orderly structures and enduring porosity extending across long distances, stand out as promising candidates for enhancing piezoelectric responses. In the pursuit of fabricating such functional structures, it is crucial to maintain non-centrosymmetry and stable electromechanical responses at periodic intervals. Particularly, it would be much easier to modify the polarization of 2D COFs. Drawing inspiration from the successful implementation of fluoropolymer-based energy harvesters, grafting fluorine-substituted alkyl chains onto COFs is able to realize piezoelectricity in these materials because their ordered structures are beneficial to dipole alignment and net spontaneous polarization. Moreover, employing these COFs in piezoelectric PENG can efficiently convert mechanical stress into electricity and facilitate the development of next-generation self-powering electronics.

### REFERENCES

All referenced literatures throughout this disclosure are incorporated herein by reference in their entirety, which include the following references:
[1] a) K. I. Park, M. Lee, Y. Liu, S. Moon, G. T. Hwang, G. Zhu, J. E. Kim, S. O. Kim, D. K. Kim, Z. L. Wang, K. J. Lee, Adv. Mater. 2012, 24, 2999-3004; b) L. Persano, C. Dagdeviren, Y. Su, Y. Zhang, S. Girardo, D. Pisignano, Y. Huang, J. A. Rogers, Nat. Commun. 2013, 4, 1633; c) R. Yang, Y. Qin, L. Dai, Z. L. Wang, Nat. Nanotechnol. 2009, 4, 34-39.
[2] S. Anwar, M. H. Amiri, S. Jiang, M. M. Abolhasani, P. R. F. Rocha, K. Asadi, Adv. Funct. Mater. 2021, 31, 2004326.
[3] a) Y. Su, W. Li, X. Cheng, Y. Zhou, S. Yang, X. Zhang, C. Chen, T. Yang, H. Pan, G. Xie, G. Chen, X. Zhao, X. Xiao, B. Li, H. Tai, Y. Jiang, L. Q. Chen, F. Li, J. Chen, Nat. Commun. 2022, 13, 4867; b) S. Peng, X. Yang, Y. Yang, S. Wang, Y. Zhou, J. Hu, Q. Li, J. He, Adv. Mater. 2019, 31, e1807722; c) H. Y. Zhang, Y. Y. Tang, Z. X. Gu, P. Wang, X. G. Chen, H. P. Lv, P. F. Li, Q. Jiang, N. Gu, S. Ren, R. G. Xiong, Science 2024, 383, 1492-1498.
[4] a) S. Hajra, M. Sahu, A. M. Padhan, I. S. Lee, D. K. Yi, P. Alagarsamy, S. S. Nanda, H. J. Kim, Adv. Funct. Mater. 2021, 31; b) S. Hajra, J. Panda, J. Swain, H.-G. Kim, M. Sahu, M. K. Rana, R. Samantaray, H. J. Kim, R. Sahu, Nano Energy 2022, 101; c) R. A. Shaukat, Q. M. Saqib, J. Kim, H. Song, M. U. Khan, M. Y. Chougale, J. Bae, M. J. Choi, Nano Energy 2022, 96, 107128.
[5] a) C. Zhang, D. Lei, C. Xie, X. Hang, C. He, H. L. Jiang, Adv. Mater. 2021, 33, e2106308; b) Y. Sun, J. Gao, Y. Cheng, Y.-W. Zhang, K. Zeng, J. Phys. Chem. C 2019, 123, 3122-3129.
[6] B. H. Moghadam, M. Hasanzadeh, A. Simchi, ACS Appl. Nano Mater. 2020, 3, 8742-8752.
[7] a) F. Kang, X. Wang, C. Chen, C.-S. Lee, Y. Han, Q. Zhang, J. Am. Chem. Soc. 2023, 145, 15465-15472; b) J. Sun, F. Kang, D. Yan, T. Ding, Y. Wang, X. i Zhou, Q. Zhang, Angew. Chem. Int. Ed. 2024, 63, e202406511; c) Q. Gu, J. Zha, C. Chen, X. Wang, W. Yao, J. Liu, F. Kang, J. Yang, Y. Y. Li, D. Lei, Z. Tang, Y. Han, C. Tan, Q. Zhang, Adv. Mater. 2024, 36, 2306414; d) S. Xu, Q. Zhang, Mater. Today Energy 2021, 20, 100635; e) J. Sun, Y. Xu, Y. Lv, Q. Zhang, X. Zhou, CCS Chem. 2023, 5, 1259-1276; f) Y. Shi, J. Yang, F. Gao, Q. Zhang, ACS Nano 2023, 17, 1879-1905.
[8] a) M. M. Yang, T. Y. Zhu, A. B. Renz, H. M. Sun, S. Liu, P. M. Gammon, M. Alexe, Nat. Mater. 2024, 23, 95-100; b) H. Abdolmaleki, A. B. Haugen, K. B. Buhl, K. Daasbjerg, S. Agarwala, Adv. Sci. 2023, 10, e2205942; c) G. Ico, A. Showalter, W. Bosze, S. C. Gott, B. S. Kim, M. P. Rao, N. V. Myung, J. Nam, J. Mater. Chem. A 2016, 4, 2293-2304.
[9] a) Y. Liu, M. Pharr, G. A. Salvatore, ACS Nano 2017, 11, 9614-9635; b) X. Peng, K. Dong, C. Ye, Y. Jiang, S. Zhai, R. Cheng, D. Liu, X. Gao, J. Wang, Z. L. Wang, Sci. Adv. 2020, 6, eaba9624; c) Z. Wen, M. H. Yeh, H. Guo, J. Wang, Y. Zi, W. Xu, J. Deng, L. Zhu, X. Wang, C. Hu, L. Zhu, X. Sun, Z. L. Wang, Sci. Adv. 2016, 2, e1600097.
[10] a) C. Fan, H. Wu, J. Guan, X. You, C. Yang, X. Wang, L. Cao, B. Shi, Q. Peng, Y. Kong, Y. Wu, N. A. Khan, Z. Jiang, Angew. Chem. Int. Ed. 2021, 60, 18051-18058; b) H. Duan, K. Li, M. Xie, J. M. Chen, H. G. Zhou, X. Wu, G. H. Ning, A. I. Cooper, D. Li, J. Am. Chem. Soc. 2021, 143, 19446-19453; c) V. Lakshmi, C. H. Liu, M. Rajeswara Rao, Y. Chen, Y. Fang, A. Dadvand, E. Hamzehpoor, Y. Sakai-Otsuka, R. S. Stein, D. F. Perepichka, J. Am. Chem. Soc. 2020, 142, 2155-2160.
[11] a) L. Li, Q. Yun, C. Zhu, G. Sheng, J. Guo, B. Chen, M. Zhao, Z. Zhang, Z. Lai, X. Zhang, Y. Peng, Y. Zhu, H. Zhang, J. Am. Chem. Soc. 2022, 144, 6475-6482; b) K. Liu, H. Qi, R. Dong, R. Shivhare, M. Addicoat, T. Zhang, H. Sahabudeen, T. Heine, S. Mannsfeld, U. Kaiser, Z. Zheng, X. Feng, Nat. Chem. 2019, 11, 994-1000; c) D. Kurandina, B. Huang, W. Xu, N. Hanikel, A. Daru, G. D. Stroscio, K. Wang, L. Gagliardi, F. D. Toste, O. M. Yaghi, Angew. Chem. Int. Ed. 2023, 62, e202307674.
[12] a) K. P. Huang, P. Lin, H. C. Shih, J. Appl. Phys. 2004, 96, 354-360; b) X. Wang, W. Wang, Y. Liu, M. Ren, H. Xiao, X. Liu, Anal. Chem. 2016, 88, 3926-3934.
[13] a) Z. Meng, R. M. Stolz, K. A. Mirica, J. Am. Chem. Soc. 2019, 141, 11929-11937; b) C. Song, J. Zheng, Q. Zhang, H. Yuan, A. Yu, W. Zhang, S. Zhang, G. Ouyang, Anal. Chem. 2023, 95, 7770-7778; c) Y. Ma, H. Yang, J. Guo, C. Sathe, A. Agui, J. Nordgren, Appl. Phys. Lett. 1998, 72, 3353-3355; d) P. V. Bakharev, M. Huang, M. Saxena, S. W. Lee, S. H. Joo, S. O. Park, J. Dong, D. C. Camacho-Mojica, S. Jin, Y. Kwon, M. Biswal, F. Ding, S. K. Kwak, Z. Lee, R. S. Ruoff, Nat. Nanotechnol. 2020, 15, 59-66; e) Y. Su, X. Rong, A. Gao, Y. Liu, J. Li, M. Mao, X. Qi, G. Chai, Q. Zhang, L. Suo, L. Gu, H. Li, X. Huang, L. Chen, B. Liu, Y. S. Hu, Nat. Commun. 2022, 13, 4181; f) J. Huang, Y. Shi, G. Z. Huang, S. Huang, J. Zheng, J. Xu, F. Zhu, G. Ouyang, Angew. Chem. Int. Ed. 2022, 61, e202206749.
[14] a) D. S. BIOVIA, 2017; b) S. Wu, M. Li, H. Phan, D. Wang, T. S. Herng, J. Ding, Z. Lu, J. Wu, Angew. Chem. Int. Ed. 2018, 57, 8007-8011; c) E. Jin, K. Geng, S. Fu, M. A. Addicoat, W. Zheng, S. Xie, J. S. Hu, X. Hou, X. Wu, Q. Jiang, Q. H. Xu, H. I. Wang, D. Jiang, Angew. Chem. Int. Ed. 2022, 61, e202115020.
[15] a) Y. Peng, Y. Huang, Y. Zhu, B. Chen, L. Wang, Z. Lai, Z. Zhang, M. Zhao, C. Tan, N. Yang, F. Shao, Y. Han, H. Zhang, J. Am. Chem. Soc. 2017, 139, 8698-8704; b) S. Bi, C. Yang, W. Zhang, J. Xu, L. Liu, D. Wu, X. Wang, Y. Han, Q. Liang, F. Zhang, Nat. Commun. 2019, 10, 2467.
[16] Q. Cao, L. Wan, Z. Xu, W. Kuang, H. Liu, X. Zhang, W. Zhang, Y. Lu, Y. Yao, B. Wang, K. Liu, Adv. Mater. 2023, 35, e2210550.
[17] a) C. F. Fitie, W. S. Roelofs, M. Kemerink, R. P. Sijbesma, J. Am. Chem. Soc. 2010, 132, 6892-6893; b) S. Cantekin, T. F. de Greef, A. R. Palmans, Chem. Soc. Rev. 2012, 41, 6125-6137.
[18] S. S. Kubakaddi, Phys. Rev. B 2004, 69, 035317.
[19] a) M. L. Xu, M. Lu, G. Y. Qin, X. M. Wu, T. Yu, L. N. Zhang, K. Li, X. Cheng, Y. Q. Lan, Angew. Chem. Int. Ed. 2022, 61, e202210700; b) S. Paul, S. Barman, A. Pal, A. Mukherjee, S. Ghosh, A. Datta, Chem. Mater. 2023, 35, 6463-6471.
[20] T. M. Guo, F. F. Gao, Y. J. Gong, Z. G. Li, F. Wei, W. Li, X. H. Bu, J. Am. Chem. Soc. 2023, 145, 22475-22482.
[21] a) K.-A. N. Duerloo, M. T. Ong, E. J. Reed, J. Phys. Chem. Lett. 2012, 3, 2871-2876; b) K. Zhao, Y. Guo, Y. Shen, Q. Wang, Y. Kawazoe, P. Jena, J. Phys. Chem. Lett. 2020, 11, 3501-3506.
[22] R. Yang, Y. Qin, C. Li, L. Dai, Z. L. Wang, Appl. Phys. Lett. 2009, 94, 022905.
[23] a) M. Li, T. Jiang, Y. Ren, H. Jiang, Nano Energy 2022, 103; b) J. Wang, Y. Li, Z. Xie, Y. Xu, J. Zhou, T. Cheng, H. Zhao, Z. L. Wang, Adv. Energy Mater. 2020, 10.
[24] G. Zhu, W. Q. Yang, T. Zhang, Q. Jing, J. Chen, Y. S. Zhou, P. Bai, Z. L. Wang, Nano Lett. 2014, 14, 3208-3213.
[25] Z. Wen, M. H. Yeh, H. Guo, J. Wang, Y. Zi, W. Xu, J. Deng, L. Zhu, X. Wang, C. Hu, L. Zhu, X. Sun, Z. L. Wang, Sci. Adv. 2016, 2, e1600097.
[26] F. R. Fan, W. Tang, Z. L. Wang, Adv. Mater. 2016, 28, 4283.
[27] Z. L. Wang, J. Song, Science 2006, 312, 242.
[28] L. Persano, C. Dagdeviren, Y. Su, Y. Zhang, S. Girardo, D. Pisignano, Y. Huang, J. A. Rogers, Nat. Commun. 2013, 4, 1633.
[29] T. M. Guo, F. F. Gao, Y. J. Gong, Z. G. Li, F. Wei, W. Li, X. H. Bu, J. Am. Chem. Soc. 2023, 145, 22475.
[30] J. Wang, H. Li, J. Liu, Y. Duan, S. Jiang, S. Yan, J. Am. Chem. Soc. 2003, 125, 1496.
[31] A. L. Wolfe, K. K. Duncan, J. P. Lajiness, K. Zhu, A. S. Duerfeldt, D. L. Boger, J. Med. Chem. 2013, 56, 6845-6857.
[32] J. J. Yu, L. Y. Zhao, Z. T. Shi, Q. Zhang, G. London, W. J. Liang, C. Gao, M. M. Li, X. M. Cao, H. Tian, B. L. Feringa, D. H. Qu, J. Org. Chem. 2019, 84, 5790-5802.
[33] Y. R. Wang, H. M. Ding, X. Y. Ma, M. Liu, Y. L. Yang, Y. Chen, S. L. Li, Y. Q. Lan, Angew. Chem. Int. Ed. 2022, 61, e202114648.
[34] S. Bera, S. Guerin, H. Yuan, J. O'Donnell, N. P. Reynolds, O. Maraba, W. Ji, L. J. W. Shimon, P. A. Cazade, S. A. M. Tofail, D. Thompson, R. Yang, E. Gazit, Nat. Commun. 2021, 12, 2634.
[35] T. M. Guo, F. F. Gao, Y. J. Gong, Z. G. Li, F. Wei, W. Li, X. H. Bu, J. Am. Chem. Soc. 202 3, 145, 22475-22482.
[36] D. S. BIOVIA, 2017.
[37] a) S. Wu, M. Li, H. Phan, D. Wang, T. S. Herng, J. Ding, Z. Lu, J. Wu, Angew. Chem. Int. Ed. 2018, 57, 8007-8011; b) L. Li, Q. Yun, C. Zhu, G. Sheng, J. Guo, B. Chen, M. Zhao, Z. Zhang, Z. Lai, X. Zhang, Y. Peng, Y. Zhu, H. Zhang, J. Am. Chem. Soc. 2022, 144, 6475-6482; c) E. Jin, K. Geng, S. Fu, M. A. Addicoat, W. Zheng, S. Xie, J. S. Hu, X. Hou, X. Wu, Q. Jiang, Q. H. Xu, H. I. Wang, D. Jiang, Angew. Chem. Int. Ed. 2022, 61, e202115020.
[38] a) G. Kresse, D. Joubert, Phys. Rev. B 1999, 59, 1758-1775; b) G. Kresse, J. Furthmuller, Phys. Rev. B 1996, 54, 11169-11186; c) G. Kresse, J. Hafner, Phys. Rev. B 1993, 47, 558-561; d) W. Kohn, L. J. Sham, Phys. Rev. 1965, 140, A1133-A1138.
[39] J. P. Perdew, J. A. Chevary, S. H. Vosko, K. A. Jackson, M. R. Pederson, D. J. Singh, C. Fiolhais, Phys. Rev. B 1992, 46, 6671-6687.
[40] a) K. Zhao, Y. Guo, Y. Shen, Q. Wang, Y. Kawazoe, P. Jena, J. Phys. Chem. Lett. 2020, 11, 3501-3506; b) X. Wu, D. Vanderbilt, D. R. Hamann, Phys. Rev. B 2005, 72, 035105.
[41] a) S. Baroni, P. Giannozzi, A. Testa, Phys. Rev. Lett. 1987, 58, 1861-1864; b) X. Gonze, Phys. Rev. A 1995, 52, 1096-1114; c) M. N. Blonsky, H. L. Zhuang, A. K. Singh, R. G. Hennig, ACS Nano 2015, 9, 9885-9891.
[42] a) M. Li, T. Jiang, Y. Ren, H. Jiang, Nano Energy 2022, 103; b) J. Wang, Y. Li, Z. Xie, Y. Xu, J. Zhou, T. Cheng, H. Zhao, Z. L. Wang, Adv. Energy Mater. 2020, 10.
[43] R. A. Shaukat, Q. M. Saqib, J. Kim, H. Song, M. U. Khan, M. Y. Chougale, J. Bae, M. J. Choi, Nano Energy 2022, 96, 107128.
[44] a) C. Fan, H. Wu, J. Guan, X. You, C. Yang, X. Wang, L. Cao, B. Shi, Q. Peng, Y. Kong, Y. Wu, N. A. Khan, Z. Jiang, Angew. Chem. Int. Ed. 2021, 60, 18051-18058; b) H. Duan, K. Li, M. Xie, J. M. Chen, H. G. Zhou, X. Wu, G. H. Ning, A. I. Cooper, D. Li, J. Am. Chem. Soc. 2021, 143, 19446-19453; c) V. Lakshmi, C. H. Liu, M. Rajeswara Rao, Y. Chen, Y. Fang, A. Dadvand, E. Hamzehpoor, Y. Sakai-Otsuka, R. S. Stein, D. F. Perepichka, J. Am. Chem. Soc. 2020, 142, 2155-2160; d) Y. Peng, Y. Huang, Y. Zhu, B. Chen, L. Wang, Z. Lai, Z. Zhang, M. Zhao, C. Tan, N. Yang, F. Shao, Y. Han, H. Zhang, J. Am. Chem. Soc. 2017, 139, 8698-8704; e) K. Liu, H. Qi, R. Dong, R. Shivhare, M. Addicoat, T. Zhang, H. Sahabudeen, T. Heine, S. Mannsfeld, U. Kaiser, Z. Zheng, X. Feng, Nat. Chem. 2019, 11, 994-1000.
[45] N. V. Venkataraman, S. Vasudevan, J. Phys. Chem. B 2002, 106, 7766-7773.
[46] a) N. M. Mackie, D. G. Castner, E. R. Fisher, Langmuir 1998, 14, 1227-1235; b) K. P. Huang, P. Lin, H. C. Shih, J. Appl. Phys. 2004, 96, 354-360; c) X. Wang, W. Wang, Y. Liu, M. Ren, H. Xiao, X. Liu, Anal. Chem. 2016, 88, 3926-3934. S62
[47] Z. Meng, R. M. Stolz, K. A. Mirica, J. Am. Chem. Soc. 2019, 141, 11929-11937.
[48] a) C. Song, J. Zheng, Q. Zhang, H. Yuan, A. Yu, W. Zhang, S. Zhang, G. Ouyang, Anal. Chem. 2023, 95, 7770-7778; b) Y. Ma, H. Yang, J. Guo, C. Sathe, A. Agui, J. Nordgren, Appl. Phys. Lett. 1998, 72, 3353-3355; c) P. V. Bakharev, M. Huang, M. Saxena, S. W. Lee, S. H. Joo, S. O. Park, J. Dong, D. C. Camacho-Mojica, S. Jin, Y. Kwon, M. Biswal, F. Ding, S. K. Kwak, Z. Lee, R. S. Ruoff, Nat. Nanotechnol. 2020, 15, 59-66; d) Y. Su, X. Rong, A. Gao, Y. Liu, J. Li, M. Mao, X. Qi, G. Chai, Q. Zhang, L. Suo, L. Gu, H. Li, X. Huang, L. Chen, B. Liu, Y. S. Hu, Nat. Commun. 2022, 13, 4181; e) J. Huang, Y. Shi, G. Z. Huang, S. Huang, J. Zheng, J. Xu, F. Zhu, G. Ouyang, Angew. Chem. Int. Ed. 2022, 61, e202206749.
[49] Y. H. Hu, L. Zhang, Phys. Rev. B 2010, 81, 174103.

### SUMMARY OF INVENTION

According to a first aspect of the invention, there is provided a two-dimensional (2D) COF compound, which includes piezoelectric monomers that are inter-connected via covalent bonds. Each of the piezoelectric monomers contains fluorinated alkyl chains tethered on peripheral positions of an aromatic moiety.

In some embodiments, the fluorinated alkyl chains tethered on the aromatic moiety contain asymmetric fluorinated alkyl chains.

In some embodiments, one of the fluorinated alkyl chains contains at least one methylene group that is substituted by fluorine.

In some embodiments, at least some of the fluorinated alkyl chains have different lengths.

In some embodiments, one of the fluorinated alkyl chains contains at least one carbon.

In some embodiments, one of the fluorinated alkyl chains is 3,3,4,4,5,5,6,6,7,7,8,8,8-trideca-fluorooctyl side chain.

In some embodiments, at least one of the fluorinated alkyl chains contains one or more difluoromethylene (—CF₂) moieties.

In some embodiments, each one of the fluorinated alkyl chains contains one or more difluoromethylene (—CF₂) moieties.

In some embodiments, the piezoelectric monomers include two or more different monomers connected to each other.

In some embodiments, the two or more different monomers includes triamino-/tetraamino-monomers in condensation reaction with dialdehyde-monomers, or diamino-monomers in condensation reaction with trialdehyde-/tetraaldehyde-monomers.

According to another aspect of the invention, there is provided a method of synthesizing the COF compound above or its variation. The method includes steps of a) providing an aromatic precursor; and b) anchoring fluorinated alkyl chains onto the aromatic precursor.

In some embodiments, the aromatic precursor includes hydroxyl groups or halogen atoms on the peripheral positions, which facilitate fluorinated reaction.

In some embodiments, Step b) further includes mixing the aromatic precursor with a fluorinated agent and a solvent to obtain a reaction mixture.

In some embodiments, the solvent is chosen from a group consisting of N,N-Dimethylformamide, 1,2-dichlorobenzene, dichloromethane, 1,2-dichloroethane, tetrahydrofuran, n-hexane, ethyl acetate, toluene, acetonitrile, or a mixture thereof.

In some embodiments, Step c) is conducted in an alkaline condition.

In some embodiments, the method further includes steps of: c) quenching the reaction mixture; d) filtering the reaction mixture; e) washing the reaction mixture; and f) drying the reaction mixture.

According to a further aspect of the invention, there is provided a piezoelectric nanogenerator device which includes: a) a top electrode; b) a bottom electrode; and c) an active layer configured between the top electrode and the bottom electrode. The active layer is fabricated using the COF compound as mentioned above or its variation.

According to a further aspect of the invention, there is provided a method of preparing an active layer for a piezoelectric nanogenerator device. The method includes the steps of: a) mixing a COF compound as mentioned above (or its variation) with a polymer material and a solvent to form a mixture; and b) drying the mixture to obtain the active layer.

According to a further aspect of the invention, there is provided a class of COF structures with alkyl side chains, which include fluorine-substituted alkyl chains.

Optionally, the alkyl side chains with various lengths are included.

Optionally, the alkyl side chain includes of one or more difluoromethylene (—CF₂) moieties.

Optionally, the alkyl side chain could be symmetry or asymmetry on the peripheral position of an aromatic moiety.

Optionally, the fully fluorine-substituted alkyl side chain is included, which comprises fluorine-substituted alkyl side chains with different lengths.

In a further aspect of the invention, there is provided a series of COFs with various backbones due to the connection patterns of different monomers which comprises of triamino-/tetraamino-monomers for condensation reaction with dialdehyde-monomers, and diamino-monomers for condensation reaction with trialdehyde-/tetraaldehyde-monomers to build two-dimensional COFs.

In a further aspect of the invention, there is provided a piezoelectric COF based nanogenerator device, comprising of a top electrode, a bottom electrode and an active layer in the middle of two electrodes. The active layer includes a piezoelectric COF with certain weight ratio in commercial matrix such as polydimethylsiloxane (PDMS), polyurethane (PU) and polyvinyl alcohol (PVA).

Optionally, metals including Al, Cu, Au and so on can be used for the top and bottom electrode. In a piezoelectric nanogenerator (PENG) the top and bottom electrodes can be the same or different as well.

Optionally, the PENG further includes flexible substrates including polytetrafluoroethylene (PTFE) and polyvinyl alcohol (PVA) for wearable devices.

In a further aspect of the invention, there is provided a method of preparing piezoelectric monomers comprising the steps of: anchoring side chain onto an aromatic precursor with alkaline agents in solution, quenching the mixture, filtration, washing and drying the cake.

Optionally, the side chain contains alkyl chains with different lengths. The side chain can be fully fluorinated. The side alkyl chain comprises at least one fluorinated group.

Optionally, the aromatic precursor comprises hydroxyl groups or halogen atoms on the peripheral positions facilitating the fluorinated reaction.

Optionally, the step of substitution reaction conducts in an alkaline condition.

Optionally, the substitution reaction uses alkaline reagents including potassium carbonate (K₂CO₃), cesium carbonate (Cs₂CO₃), pyridine, triethylamine (Et₃N) and so on.

Optionally, the step of fluorinated reaction comprises mixing an alkaline reagent and a solvent with the aromatic precursor and alkyl chain reactant to form the mixture, the solvent comprising any one of N,N-dimethylformamide (DMF), dimethylacetamide (DMAc), acetone, acetonitrile (CH3CN), 1,2-dichloromethane (DCM), chloroform (CHCl₃), ethyl acetate (EA), tetrahydrofuran (THF), n-hexane or a mixture there of.

Optionally, the step of washing includes using water, N,N-dimethylformamide (DMF), dichloromethane (DCM), chloroform (CHCl₃), ethyl acetate (EA), tetrahydrofuran (THF) and n-hexane.

In a further aspect of the invention, there is provided a method of fabricating a PENG device, comprising the steps of: assembling components of the PENG device within a larger case and a smaller case in a predetermined order, and packing the larger case and the smaller case together with a polymer (including polyethylene glycol terephthalate (PET), polyurethane (PU), polyimide (PI), polyethylene oxide (PEO) and polyethylene (PE)) for encapsulation.

Exemplary embodiments of the invention thus provide structures and synthesis of a class of piezoelectric monomers and COFs with fluorine-substituted side chains tethered on the peripheral position of an aromatic moiety (thus different functional side chains). The multi-fluoro groups on the mentioned piezoelectric COFs provide abundant piezoelectric active sites, which enable the application as the active layer for PENGs. The exemplary embodiments also provide a process for the fabrication of a piezoelectric COF and a process for the fabrication of the piezoelectric nanogenerator utilizing mentioned piezoelectric COF. There are also disclosed structures and synthesis of a class of piezoelectric COFs containing a different backbone with various connection patterns (thus different COF backbones). Fabrication of the PENG based on a mentioned piezoelectric COF is also disclosed. One of the main applications is using the prepared 2D COF as the functional material of the active layer for PENG.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

### BRIEF DESCRIPTION OF FIGURES

The foregoing and further features of the present invention will be apparent from the following description of embodiments which are provided by way of example only in connection with the accompanying figures, of which:

FIG. 1 illustrates several Fourier transform infrared (FT-IR) spectra obtained from characterization of the piezoelectric monomers and the reactant according to an embodiment of the invention.

FIG. 2 depicts the ¹H and ¹⁹F nuclear magnetic resonance (NMR) spectra obtained from characterization of the piezoelectric monomers.

FIG. 3 illustrates several FT-IR spectra obtained from characterization of the benzene core based piezoelectric monomers and corresponding COFs.

FIG. 4 shows several FT-IR spectra obtained from characterization of the triazine core based piezoelectric monomers and corresponding COFs.

FIG. 5 illustrates several of X-Ray photoelectron spectra (XPS) including C1s, N1s, O1s and F1s regions obtained from characterization of the benzene core based piezoelectric COFs.

FIG. 6 illustrates several XPS spectra including C1s, N1s, O1s and F1s regions obtained from characterization of the triazine core based piezoelectric COFs.

FIG. 7 illustrates a number of powder X-ray diffraction (PXRD) patterns obtained from characterization of COFs with benzene and triazine cores.

FIG. 8 shows the high-resolution transmission electron microscopy (HRTEM) images with clear lattice labelled for COFs with benzene and triazine cores.

FIG. 9 shows the atomic force microscopy (AFM) images of COFs with benzene and triazine cores.

FIG. 10 shows the topographic images, vertical PFM amplitudes and corresponding phase images with amplitude butterfly loops and piezoresponse phase hysteresis loops for benzene core COFs.

FIG. 11 shows the topographic images, vertical PFM amplitudes and corresponding phase images with amplitude butterfly loops and piezoresponse phase hysteresis loops for triazine core COFs.

FIG. 12 shows the Kelvin probe force microscopy (KPFM) surface potential images of COFs with benzene core and the corresponding surface potential curves along the line indicating dramatic differences in the surface potential compared to the substrate.

FIG. 13 shows the KPFM surface potential images of COFs with triazine core and the corresponding surface potential curves along the line indicating dramatic differences in the surface potential compared to the substrate.

FIG. 14 shows the high piezoelectric constant (d₃₃) value of piezoelectric COFs with benzene and triazine cores implying their high piezoresponse.

FIG. 15 is a schematic illustration of the piezoelectric nanogenerator (PENG) device and the optical photograph of the PENG device after encapsulating with polymers for testing.

FIG. 16 shows the output open-circuit voltage (V_{oc}) of PENG fabricated using piezoelectric COFs with benzene core as well as the V_{oc} of PENG with both the forward and reverse connections (polarity switching test).

FIG. 17 is a graph of output short circuit current (I_{sc}) of PENG and variation of I_{sc} or power with load resistance for PENG based on piezoelectric COFs with benzene core.

FIG. 18 shows the stability curves over time up to 600 s for PENG based on piezoelectric COFs with benzene core.

FIG. 19 shows the long-term stability of the PENG device based on piezoelectric COFs with benzene core for 14 days.

FIG. 20 shows the output open-circuit voltage (V_{oc}) of PENG fabricated using piezoelectric COFs with triazine core as well as the V_{oc} of PENG with both the forward and reverse connections (polarity switching test).

FIG. 21 is a graph of output short circuit current (I_{sc}) of PENG and variation of I_{sc} or power with load resistance for PENG based on piezoelectric COFs with triazine core.

FIG. 22 shows the stability curves over time up to 600 s for PENG based on piezoelectric COFs with triazine core.

FIG. 23 shows the long-term stability of the PENG device based on piezoelectric COFs with triazine core for 14 days.

FIG. 24 shows the photograph of PENG fabricated using piezoelectric COFs successfully driving commercial light emitting diodes (LEDs) arrays showing word “CityU-SDU” and the photograph of PENG successfully driving the LED display timer.

FIG. 25: a) Schematic representation for the synthesis of CityU-13 and CityU-14. b) Top and side views of refined AA-stacking structures of CityU-13 and CityU-14, where hydrogen atoms are ignored for simplification. c) Average background subtraction filter-denoised HRTEM images of CityU-13 and CityU-14. The lattice spacing and corresponding planes are labelled. Insets are the corresponding fast Fourier transform (FFT) patterns.

FIG. 26 shows synthetic routes to 13F-OTs and 13F—CHO.

FIG. 27: (a) The 1H NMR spectrum of 13F-OTs in CDCl₃. (b) The 19F NMR spectrum of 13F-OTs in CDCl₃. The corresponding H and F in particular positions were labelled in the molecular structure.

FIG. 28: (a) The FTIR spectra of DHTP and 13F—CHO. The dramatic attenuation of OH signal (3262 cm⁻¹) indicates the efficient reaction between DHTP and 13F-OTs. (b) The enlarged FTIR spectra of DHTP and 13F—CHO from 500 to 1500 cm⁻¹, where C—F signals (1200 cm⁻¹) could be clearly found.

FIG. 29: (a) The ¹H NMR spectrum of 13F—CHO in CDCl₃. (b) The ¹⁹F NMR spectrum of 13F—CHO in CDCl₃. The corresponding H and F in particular positions were labelled in the molecular structure.

FIG. 30 illustrates the synthetic routes to CityU-13 and CityU-14.

FIG. 31: (a) FTIR spectra of CityU-13 with TAPB and 13F—CHO for comparison. (b) FTIR spectra of CityU-14 with corresponding precursors TAPT and 13F—CHO for comparison. The highlighted range in the spectra clearly shows the change of signals from precursors compared to that from corresponding COFs. The attenuation of signals from amino group (3420, 3345, and 3205 cm⁻¹ for TAPB and 3460, 3320, and 3205 cm⁻¹ for TAPT) and aldehyde group (1680 cm⁻¹) demonstrate the successful condensation reaction between them with the formation of imine bond (1620 cm⁻¹).[37b, 44] The insets are the enlarged spectra that clearly show the signals from alkyl groups (2960, 2920, 2875, and 2850 cm⁻¹) in the side chains.[45](c) The enlarged FTIR spectra of CityU-13 in the range of 500-1500 cm⁻¹ with corresponding precursors TAPB and 13F—CHO for comparison. (d) The enlarged FTIR spectra of CityU-14 in the range of 500-1500 cm⁻¹ with corresponding precursors TAPT and 13F—CHO for comparison. The fluorinated side chains were retained after the condensation reaction due to the retention of alkyl and CF2 (1200 cm⁻¹) signals.[46]

FIG. 32 shows the total XPS spectra of (a) CityU-13 and (b) CityU-14. The F elements in the side chain could be clearly found in the survey spectra of two COFs, indicating the fluorinated alkyl chain was retained during the construction of COFs through condensation reaction. In addition, the atomic ratio of C/N/O/F in CityU-13 is 60.11/2.56/3.67/33.66, which is close to that from simulated structure (C/N/O/F=50.18/2.39/2.73/42.12). The atomic ratio of C/N/O/F in CityU-14 is 55.09/6.31/4.75/33.86, which is close to that from the simulated structure (C/N/O/F=46.99/5.96/2.72/42.01).

FIG. 33 shows XPS spectra of CityU-13: (a) C is. (b) N is. (c) O is. (d) F is. Different colors show the attributions of different signals. C═N peak (286.9 eV) in C1s spectra indicates the formation of imine bond after condensation reaction between 13F—CHO and TAPB. C═N signal in N1s spectra (399.1 eV) also indicates the formation of imine bond after condensation reaction.[47] C—O peak in O1s spectra shows the linkage of fluorinated side chain after reaction between 13F-OTs and DHTP. The pronounced C—F₂ (291.7 eV) and C—F₃ (294.0 eV) signals in C1s spectra and obvious C—F (689.2 eV), C—F₂ (690.6 eV) and C—F₃ (691.9 eV) peaks in F is spectra imply the fluorinated alkyl chain in CityU-13. [46a, 48]

FIG. 34 shows the XPS spectra of CityU-14: (a) C 1s. (b) N 1s. (c) O 1s. (d) F 1s. Different colors show the attributions of different signals. C═N peak (287.2 eV) in C1s spectra indicates the formation of imine bond after condensation reaction between 13F—CHO and TAPT. C═N signal in N1s spectra (398.9 eV) also indicates the formation of imine bond after condensation reaction.[47] C—O peak in O1s spectra shows the linkage of fluorinated side chain after reaction between 13F-OTs and DHTP. The pronounced C—F₂ (291.8 eV) and C—F₃ (293.8 eV) signals in C1s spectra and obvious C—F (689.1 eV), C—F₂ (690.7 eV) and C—F₃ (692.2 eV) peaks in F is spectra imply the fluorinated alkyl chain in CityU-14. [46a, 48]

FIG. 35 illustrates SEM image and elemental mappings of CityU-13 and CityU-14. Elemental mappings exhibit uniform distribution of different elements within both COFs.

FIG. 36 shows EDS spectra of CityU-13 and CityU-14. Both of them exhibit conspicuous F signals.

FIG. 37 shows the diffraction patterns of (a) CityU-13 and (b) CityU-14 with Pawley refined results and simulated AA-stacking models as comparison. The experimental PXRD patterns are well consistent with the simulated results with an AA-stacking model. The unit cell parameters of both COFs were iteratively optimized with the convergence of R_{wp} and Rₚ. The small differences and satisfied agreement factors (R_{wp} and Rₚ) after Pawley refinement clearly confirm the periodic structures with the satisfied crystallinity for both COFs.

FIG. 38 illustrates TEM images of (a) CityU-13 and (b) CityU-14; and HRTEM images of (c) CityU-13 and (d) CityU-14. The pronounced lattice fringes of both COFs could be found in HRTEM images, indicating their ordered structure with high crystallinity. The region in the white dashed box was selected for Average background subtraction filter-denoised HRTEM images.

FIG. 39 shows nitrogen adsorption (ADS) and desorption (DES) isotherms linear plots of (a) CityU-13 and (b) CityU-14 collected at 77 K. The Brunauer-Emmett-Teller (BET) surface areas of both COFs are close to each other due to their similar structure. In addition, FIG. 39 shows the pore size distribution based on NLDFT calculation for (c) CityU-13 and (d) CityU-14. The pore size of CityU-13 (3.0 nm) is close to that of the optimized structure with an AA-stacking model, where the pore size is ˜3.0 nm. The pore size of CityU-14 (2.7 nm) is also close to that of the optimized structure with the pore size of around 3.0 nm.

FIG. 40 shows water contact angles of (a) CityU-13 and (b) CityU-14. The large water contact angles of both COFs are ascribed to the introduction of fluorinated alkyl chains with high hydrophobicity.

FIG. 41 shows TGA curves of (a) CityU-13 and (b) CityU-14. The value labelled here indicates the temperature at which the weight loss occurs. It is worth noting that both COFs are stable up to 300° C. without remarkable weight loss.

FIG. 42 shows FTIR spectra of (a) CityU-13 and (b) CityU-14 after a long period of time up to 14 days at room temperature. The negligible change in the signals, especially the peak at 1618 cm⁻¹ that are attributed to imine-linkage clearly demonstrate the stability of frameworks.

FIG. 43 shows the Raman spectra of (a) CityU-13 and (b) CityU-14 after a long period of time up to 14 days at room temperature. The strong Raman bands around 1600 cm-1 can be primarily attributed to aromatic benzene rings in frameworks. [43, 49] The negligible change in the signals also demonstrate the stability of frameworks.

FIG. 44 illustrates PXRD patterns of (a) CityU-13 and (b) CityU-14 after a long period of time up to 14 days at room temperature. The strong diffraction peaks even after 14 days provide solid evidence of the good stability.

FIG. 45 shows the total energy as a function of the AIMD simulation time for CityU-13 and CityU-14 monolayer at 300 K.

FIG. 46 illustrates 2D and 3D Location dependent Raman mapping of (a) CityU-13 and (b) CityU-14 with size of 20×20 μm². The laser excitation is 633 nm.

FIG. 47 illustrates topographic images of (a) CityU-13 and (b) CityU-14.

FIG. 48: a) Vertical PFM amplitudes and corresponding amplitude butterfly loops of CityU-13 and CityU-14. b) PFM phase images and corresponding piezoresponse phase hysteresis loops of CityU-13 and CityU-14. c) KPFM surface potential images and the corresponding surface potential curves along the black line indicating dramatic differences compared to the Al substrate.

FIG. 49 shows (a, b) Lateral PFM amplitudes and phase images of CityU-13; and (c, d) Corresponding amplitude butterfly loops and piezoresponse phase hysteresis loops.

FIG. 50 shows (a, b) Lateral PFM amplitudes and phase images of CityU-14; and (c, d) Corresponding amplitude butterfly loops and piezoresponse phase hysteresis loops.

FIG. 51 shows piezoelectric coefficient (d₃₃) measurement of (a, b) CityU-13 and (c, d) CityU-14 using quasi-static (Berlincourt) method. The high d₃₃ value of both COFs implies their high piezo response.

FIG. 52 shows AFM topological images of (a) CityU-13 and (b) CityU-14.

FIG. 53 includes (a) surface potential images of CityU-13 and CityU-14 by KPFM characterization; and (b) Surface potential curves along the white line.

FIG. 54: a) Schematic diagram of a piezoelectric energy harvester with Al/active layer/Al architecture. b) Output open-circuit voltage (V_{oc}) of PENG based on CityU-13. c) V_{oc} of PENG based on CityU-13 with both the forward and reverse connections. d) Output short circuit current (I_{sc}) of CityU-13 based PENG. e) Variation of I_{sc} and power with load resistance for PENG based on CityU-13. The inset shows a photo of PENG. f) Photograph of CityU-13 based PENG successfully driving 76 commercial light emitting diodes (LEDs) showing word “CityU-SDU”. g) Photograph of CityU-13 based PENG successfully driving the LED display timer. h) Stability tests over time up to 600 s for PENG based on CityU-13.

FIG. 55 shows the cross-sectional SEM and EDS mapping images of COF based active layer and bottom Al electrode. The obvious Al signal at the top section while the pronounced C/F information at the bottom demonstrates the sandwich structure of PENG with Al electrode and COF based active layer.

FIG. 56 are the cross-sectional SEM images of COF based active layer and top Al electrode. The COFs active layer was closely attached onto the Al electrode without an obvious air gap.

FIG. 57: (a) Output open-circuit voltage (V_{oc}) of CityU-14 based PENG. (b) V_{oc} of CityU-14 based PENG with both the forward and reverse connections. (c) Output short circuit current (I_{sc}) of CityU-14 based PENG. The I_{sc} of CityU-13 and CityU-14 are close due to their similar structure, polarity and piezoelectric coefficients. (d) Variation of I_{sc} and power with load resistance for PENG based on CityU-14. The inset shows the photo of PENG based on CityU-14. (e) Stability tests over time up to 600 s for PENG based on CityU-14. The output open circuit voltages of both COFs-based PENGs were retained during 600 s without dramatic fluctuation, demonstrating their excellent stabilities.

FIG. 58 shows variation of V_{oc} and power with load resistance for PENG based on (a) CityU-13 and (b) CityU-14.

FIG. 59 is a side view photo of a piezoelectric nanogenerator driving the 76 LEDs showing the word “CityU-SDU”. COFs-based PENG can successfully power up the array of 76 LEDs, proving the practical applications of COFs-based PENG.

FIG. 60 shows the long-term stability of the PENG device based on (a) CityU-13 and (b) CityU-14 for 14 days.

FIG. 61 shows comparison of the output open circuit voltage between COFs in this work and other reported organic piezoelectrics.

FIG. 62: a) DFT simulation of dipole moments for CityU-13 and CityU-14. The arrow shows the conventional direction of the dipole moment vector. b) Schematics of dipoles alignment and net spontaneous polarization in COFs without stress. c) Schematic illustration of piezoelectricity with electric pulse due to the separation of positive/negative charges upon stress.

### DETAILED DESCRIPTION OF PREFERRED EMBODIMENTS

Before any embodiments of the invention are explained in detail, it is to be understood that the invention is not limited in its application to the details of construction and the arrangement of components set forth in the following description or illustrated in the following drawings. The invention is capable of being embodied in other forms and of being practiced or of being carried out in various ways. Also, it is to be understood that the phraseology and terminology used herein is for the purpose of description and should not be regarded as limiting. Terms of degree, such as “substantially” or “about” are understood by those of ordinary skill to refer to reasonable ranges outside of the given value, for example, general tolerances associated with manufacturing, assembly, testing, and use of the described embodiments.

The collective contribution from the nanostructures with high crystallinity and ordered arrangement (dipole ordering) will enhance the performance of devices. Moreover, it has been shown that energy harvesters based on porous PVDF-TrFE films as well as nanofibers, with pores confined inside the polymer, show enhanced output power of the nanogenerator. In this context, isoreticular COFs have drawn increasing attention due to the permanent porosity, high crystallinity and ordered structure. These promising properties make COFs satisfying candidates for diverse applications including adsorption, catalysis and battery. Importantly, the inherent connection patterns and wide tunability in unsymmetrical building units allow developing COFs with noncentrosymmetric crystal structure, which is a pivotal prerequisite to the piezoelectric effect. Such effects with polarization and dipole moment appear in COFs due to a charge-transfer process under an applied mechanical force. In addition, fluorine plays a significant role in the piezoelectric effect. Aiming to improve the piezoelectric energy conversion efficiency, fluorine-containing alkyl chain as a promising structural motif could be introduced into highly ordered COFs for intrinsic polarizability and enhanced charge transfer. Therefore, development of piezoelectric COFs is a promising way to improve the performance of piezoelectric nanogenerators (PENG) and is highly demanded.

Herein, a series of imine-linked 2D COFs with fluorine-substituted side chains is provided to trigger the asymmetry, intrinsic polarizability and piezoelectric properties. Applying them in PENGs, superb output open circuit voltage, short circuit current and long stability under an applied force could be obtained.

The tunable structures of piezoelectric COF by judicious design of the building units enable diversity of piezoelectric COFs based on the connection patterns and functionalization of side chains (implantation of fluorochains). Moreover, the post-treatment of the side chain will also introduce piezoelectric properties into COFs. Therefore, the adjustment of piezoelectric COF can be easy to be implemented.

In one embodiment, there is provided a class of aldehyde monomers that includes several fluorinated alkyl groups tethered on the peripheral position of an aromatic moiety. As used herein, “fluorinated” refers to fluorine-rich aromatic compounds with asymmetric, symmetric forms through the substitution reaction of hydroxyl with fluorinated alkyl chain. In one embodiment, modifying the length of the alkyl chain and changing the number of fluorine substituted groups help to tune the piezoelectric properties. To be specific, the number of carbons in the alkyl chain could be at least one; the alkyl chain could contain no fluorine or be fully fluorinated or contain partial fluorinated segments with at least one fluorinated methylene. In one embodiment, modifying the backbone of the monomers could also tune the piezoelectric properties.

As will be appreciated by those skilled in the art, the synthetic methods of piezoelectric monomers and COFs are concise and efficient. Piezoelectric COFs are chemically robust with permanent porosity, and show pronounced spontaneous polarity for piezoelectricity. As such, the piezoelectric COFs may be applied as active materials in organic piezoelectric nanogenerators (PENGs) for self-powered systems.

In some embodiments, the piezoelectric monomer may be prepared from an aromatic precursor and a fluorinated agent.

The aromatic precursor may contain any substitutes of hydrogen atoms and halogen groups on the peripheral positions fascinating the fluorine substitution reaction, such as hydroxyl, bromine or iodine groups. For example, one fluorinated substituted precursor is 2,5-Dihydroxyterephthalaldehyde.

The fluorinated agent is a fluorine-contained alkyl chain. The number of carbon with fluorine anchored may be at least one. For example, one fluorinated agent is 3,3,4,4,5,5,6,6,7,7,8,8,8-tridecafluorooctan-1-ol (13F—OH).

The method of preparing the fluorinated monomers may including the steps of: activating fluorinated agent with 4-toluenesulfonyl chloride, and then anchoring fluorinated agent on an aromatic precursor in solution, quenching the mixture, filtration, washing and drying the cake.

The step of fluorinated substitution comprises mixing a fluorinated agent and a solvent with the aromatic precursor to form the mixture, the solvent comprising any one of N,N-dimethylformamide, 1,2-dichlorobenzene, dichloromethane, 1,2-dichloroethane, tetrahydrofuran, acetone, n-hexane, ethyl acetate, toluene, or acetonitrile or a mixture thereof.

The step of fluorinated substitution reaction conducts in an alkaline condition.

The alkaline condition includes any of one potassium carbonate, cesium carbonate, triethylamine or pyridine.

Upon completion of reaction, the reaction mixture may be quenched by the addition of a suitable solution or solvent. Such solvent may be an aqueous or an organic solvent known in the art. Non-limiting examples of such solvent include water, dichloromethane, chloroform, ethyl acetate and the organic solvent as defined above.

Solution form of the fluorinated product obtained after the above may be separated from the reaction mixture via a suitable separation techniques known in the art. Non-limiting examples of such separation technique include extraction, filtration, centrifugation and decantation. The resulting solid product may be optionally subjected to drying at about room temperature to about 50-70° C., about 70-90° C., or about 90-110° C.

The drying above may be undertaken optionally under vacuum for a period of about 4 hours to 6 hours, about 6 hours to 8 hours, about 8 hours to 12 hours, about 8 hours to 14 hours, about 10 hours to 12 hours.

Once the fluorinated precursor and COFs above are recovered, the fluorinated precursor and COFs may be subjected for material characterization. The non-limiting examples of such characterization include Fourier transform infrared (FT-IR), nuclear magnetic resonance (NMR), X-ray diffraction (XRD), UV-Visible absorption spectroscopy (UV-Vis), X-ray Photoelectron Spectroscopy (XPS), High-resolution transmission electron microscopy (HRTEM) and suitable methods to evaluate the electrochemical properties of the composition described herein.

In the present disclosure, there is provided the exemplary conditions for preparing the fluorinated monomers and COFs as defined above. The exemplary conditions are provided for illustration purposes only and it is to be understood that the conditions are not limited to these exemplary conditions.

### Exemplary Condition 1

When hydroxyl-substituted aromatic precursor and p-toluensulfonyl substituted fluorinated agent are mixed at a mole ratio of 1:2 or 1:3 or 1:4 or 1:5 or 1:6 or 1:7 or 1:8 or 1:9 or 1:10, in the presence of polar solvents such as N-methylpyrrolidone, dimethylformamide and acetonitrile under argon or nitrogen atmosphere, followed by stirring at 80-120° C. for about 8-24 hours, the fluorinated chain may be anchored and may be represented by the following structures.

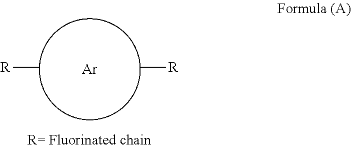

### Exemplary Condition 2

When hydroxyl-substituted aromatic precursor and p-toluensulfonyl substituted fluorinated agent are mixed at a mole ratio of 1:3 or 1:4 or 1:5 or 1:6 or 1:7 or 1:8 or 1:9 or 1:10, in the presence of polar solvents such as N-methylpyrrolidone, dimethylformamide and acetonitrile under argon or nitrogen atmosphere, followed by stirring at 80-120° C. for about 8-24 hours, the fluorinated chain may be anchored and may be represented by the following structures.

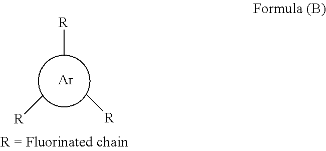

### Exemplary Condition 3

When hydroxyl-substituted aromatic precursor and p-toluensulfonyl substituted fluorinated agent are mixed at a mole ratio of 1:4 or 1:5 or 1:6 or 1:7 or 1:8 or 1:9 or 1:10, in the presence of acetonitrile under argon or nitrogen atmosphere, followed by stirring at 80-120° C. for about 8-24 hours, the fluorinated chain may be anchored and may be represented by the following structures.

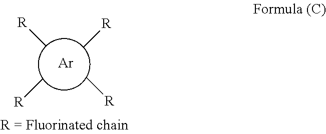

### Exemplary Condition 4

When fluorinated aldehyde monomer and the amino monomer are mixed at a mole ratio of 3:2, in the presence of 1,2-dichlorobenzene, n-butanol, mesitylene and 1,4-dioxane with the 6M acetic acid as catalyst in the pyrex tube, followed by several cycles of freeze-pump-thaw, the mixture in pyrex tube is heated at 100-150° C. for about 3-7 days. Besides, mesitylene and dioxane can also be employed as the solvents. The COF with fluorinated chains may be represented by the following structures.

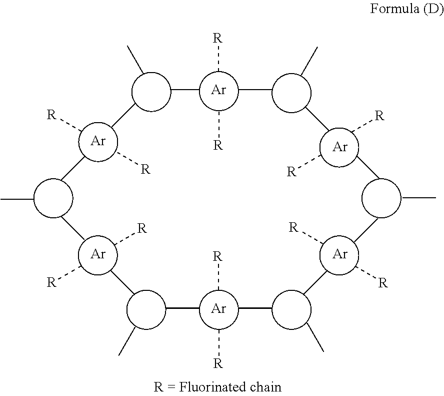

### Exemplary Condition 5

When fluorinated aldehyde monomer and the amino monomer are mixed at a mole ratio of 2:1, in the presence of 1,2-dichlorobenzene, n-butanol, mesitylene and 1,4-dioxane with the 6M acetic acid as catalyst in the pyrex tube, followed by several cycles of freeze-pump-thaw, the mixture in pyrex tube is heated at 100-150° C. for about 3-7 days. Besides, mesitylene and dioxane can also be employed as the solvents. The COF with fluorinated chains may be represented by the following structures.

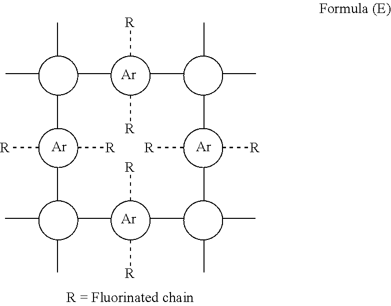

As can be seen from the exemplary conditions and the examples provided in the present disclosure, the process for preparing piezoelectric COFs with fluorinated alkyl chains as described herein is undertaken in mild and simple conditions. Hence, this method may be scaled-up in a straightforward manner.

As discussed above, the piezoelectric COFs with fluorinated alkyl chains may be used in mechanical energy harvesting applications. More specifically, the CH₂/CF₂ dipoles provided by the side chains generate dipole moments and the separation of positive/negative charges upon mechanical stress facilitates the piezoelectricity with electrical pulse (output open circuit voltage). Especially, the ordered arrangement of COF structures with alignment of pores will lead to the alignment of dipoles in the side chain for enhanced piezoelectric response. Therefore, the collective effect of fluorinated alkyl chain and the ordered COFs structure can be perfectly combined to produce good piezoelectric properties.

Exemplary, non-limiting embodiments of a piezoelectric nanogenerator (PENG) comprising a piezoelectric active layer disclosed herein and method of making said COF based PENG, will now be disclosed.

There is provided a PENG comprising a top and bottom electrode with COFs as the active layer in the middle. The piezoelectric active layer comprising a piezoelectric COF may be prepared by mixing the COF with a matrix material that may include polydimethylsiloxane (PDMS), polyvinyl pyrrolidone (PVP) or other polymer materials in an appropriate ratio.

The method of mixing the active material i.e. piezoelectric COFs defined herein with the matrix material stated above may be further mixed in the presence of a solvent, and the thus-obtained paste may be coated on a substrate such as a glass, stainless steel or aluminum sheet using a coater.

The solvent may be then removed under vacuum at about 60° C., about 70° C., about 80° C., about 90° C., about 100° C., about 110° C., or about 120° C. for about 6 to 9 hours, about 6 to 12 hours, about 6 to 16 hours, about 9 to 12 hours, about 9 to 16 hours, preferably for about 10 hours.

The active layer as described herein may be assembled in a hermetically sealed two electrode system and this system is used to further evaluate the electrochemical performance after wrapping with a polymer such as polyethylene terephthalate (PET) as described herein. The thickness of the active layer could be around 10-30 μm, 30-60 μm, 60-90 μm, 90-120 μm, 120-150 μm, 150-180 μm, 180-210 μm, 210-240 μm, 240-270 μm and 270-300 μm.

Unless otherwise specifically provided, all tests herein are conducted at standard conditions which include a room and testing temperature of 25° C., sea level (1 atm.) pressure, and all measurements are made in metric units. Furthermore, all percentages, ratios, etc. herein are by weight, unless specifically indicated otherwise. It is understood that unless otherwise specifically noted, the materials, chemicals, etc. described herein are typically commodity items and/or industry-standard items available from a variety of suppliers worldwide. All the reagents and solvents are used without further purification unless otherwise specified. 1,4-dioxane, mesitylene, n-butanol and 1,2-dichlorobenzene are purchased as extra dry grade with water lower than 50 ppm.

As mentioned above, when used, the PENG comprising a COF based active layer as described above may exhibit high output open circuit voltage, high output short circuit current, high output power and long cycling period. Therefore, advantageously, the COFs with fluorinated substituted chain as defined herein may potentially be used as active layers for high-performance PENG.

TABLE 1
The amino monomers

| | |
|---|---|
| 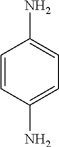 | I |
| 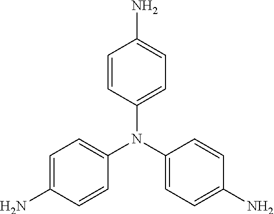 [SMILES: Nc1ccc(N(c2ccc(N)cc2)c2ccc(N)cc2)cc1] | II |
| 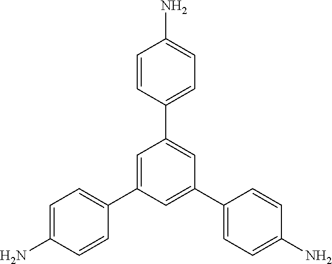 [SMILES: Nc1ccc(-c2cc(-c3ccc(N)cc3)cc(-c3ccc(N)cc3)c2)cc1] | III |
| 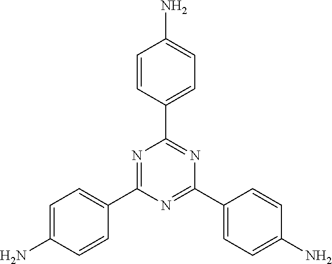 [SMILES: Nc1ccc(-c2nc(-c3ccc(N)cc3)nc(-c3ccc(N)cc3)n2)cc1] | IV |
| 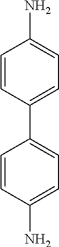 [SMILES: Nc1ccc(-c2ccc(N)cc2)cc1] | V |
| 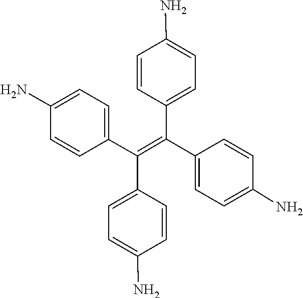 [SMILES: Nc1ccc(C(=C(c2ccc(N)cc2)c2ccc(N)cc2)c2ccc(N)cc2)cc1] | VI |
| 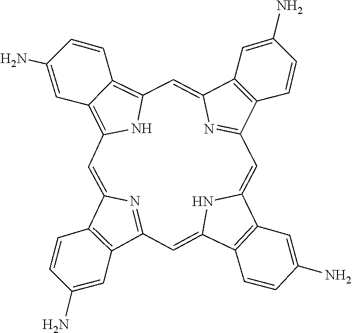 [SMILES: Nc1ccc2c(c1)-c1cc3[nH]c(cc4nc(cc5[nH]c(cc-2n1)c1cc(N)ccc51)-c1cc(N)ccc1-4)c1cc(N)ccc31] | VII |
| 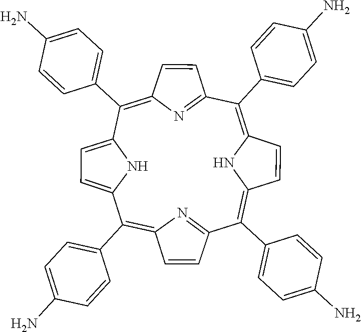 [SMILES: Nc1ccc(-c2c3nc(c(-c4ccc(N)cc4)c4ccc([nH]4)c(-c4ccc(N)cc4)c4nc(c(-c5ccc(N)cc5)c5ccc2[nH]5)C=C4)C=C3)cc1] | VIII |

### Exemplary Compounds Synthesized by the Methods of the Invention

Exemplary compounds that can be synthesized by the methods of the present invention are depicted in Tables 1 and 2 any of the monomers and their COFs after condensation reactions are acceptable.

TABLE 2
The aldehyde monomers

| | |
|---|---|
| 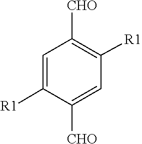 | I |
| 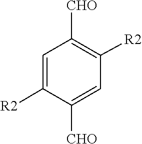 | II |
| 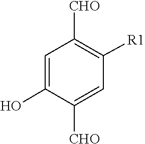 | III |
| 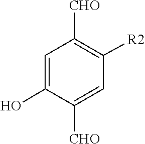 | IV |
| 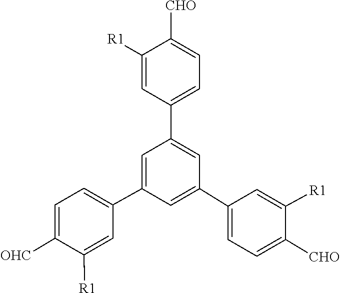 | V |
| 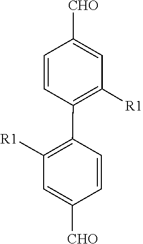 | VI |
| 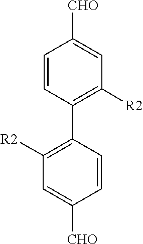 | VII |
| 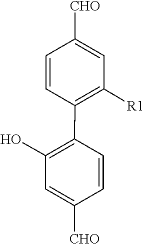 | VIII |
| 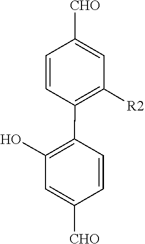 | IX |
| 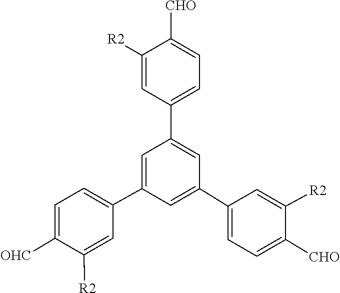 | X |

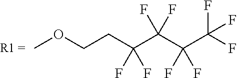 [SMILES: COCCC(F)(F)C(F)(F)C(F)(F)C(F)(F)F]

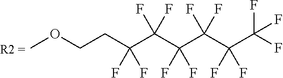 [SMILES: COCCC(F)(F)C(F)(F)C(F)(F)C(F)(F)C(F)(F)C(F)(F)F]

### Example 1: The Preparation of I

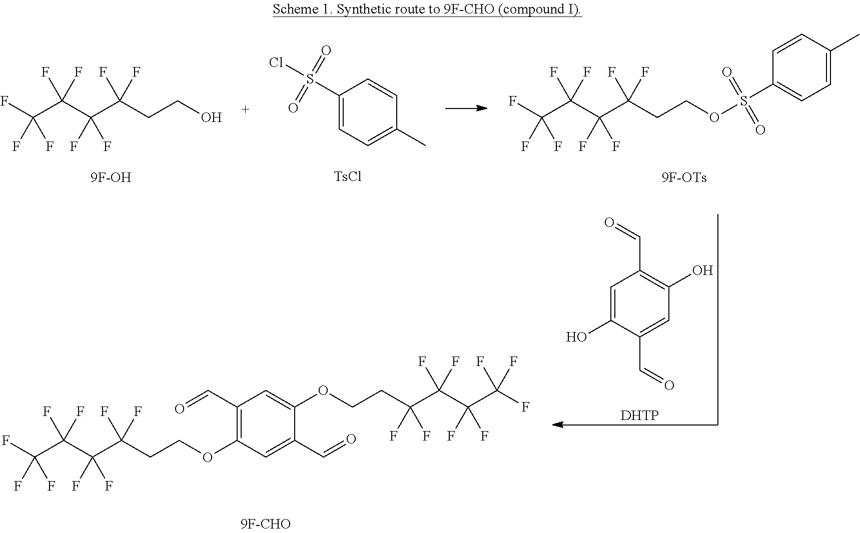

560 mg (13.76 mmol) of NaOH was loaded in the round bottom flask (RBF) before adding 16 mL of DI water and 16 mL of THF. After cooling down in the ice bath, 1.593 mL (9.6 mmol) of 9F—OH was injected to form solution 1.

Solution 2 was prepared by dissolving 1.664 g (8.96 mmol) of TsCl in 16 mL of THF. Then, solution 2 was dropwise added into solution 1. The resulting mixture was stirred for 8 hours before pouring into ice water. The white powder was harvested in 70% yield after filtration and dried under vacuum.

After obtaining 9F-OTs, 14 mg (0.084 mmol) of DHTP, 206 mg (0.492 mmol) of 9F-OTs and 243 mg of Cs₂CO₃ (0.748 mmol) were loaded into the RBF before three cycles of vacuum-N₂ purge. After injecting 10 mL of superdry CH₃CN into the RBF, the mixture was refluxed for 12 hours before extracting with dichloromethane (DCM), brine and DI water subsequently. The organic layer was collected, combined and evaporated. The resulting mixture was purified by column chromatography using the mixed solvent of DCM and n-hexane (1:2 in v/v) as eluent to afford pale yellow 9F—CHO (compound I).

### Example 2: The Preparation of II

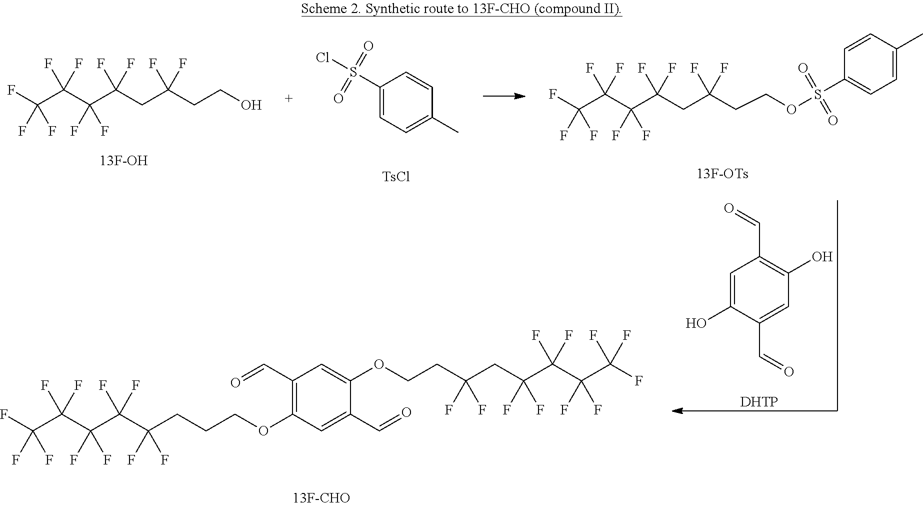

560 mg (13.76 mmol) of NaOH was loaded in the round bottom flask (RBF) before adding 16 mL of DI water and 16 mL of THF. After cooling down in the ice bath, 2.072 mL (9.6 mmol) of 13F—OH was injected to form solution 1.

Solution 2 was prepared by dissolving 1.664 g (8.96 mmol) of TsCl in 16 mL of THF. Then, solution 2 was dropwise added into solution 1. The resulting mixture was stirred for 8 hours before pouring into ice water. The white powder was harvested in 70% yield after filtration and dried under vacuum.

After obtaining 13F-OTs, 14 mg (0.084 mmol) of DHTP, 255 mg (0.492 mmol) of 13F-OTs and 243 mg of Cs₂CO₃ (0.748 mmol) were loaded into the RBF before three cycles of vacuum-N₂ purge. After injecting 10 mL of superdry CH₃CN into the RBF, the mixture was refluxed for 12 hours before extracting with dichloromethane (DCM), brine and DI water subsequently. The organic layer was collected, combined and evaporated. The resulting mixture was purified by column chromatography using the mixed solvent of DCM and n-hexane (1:2 in v/v) as eluent to afford pale yellow 13F—CHO (compound II).

### Example 3: Materials Characterization

a. FT-IR

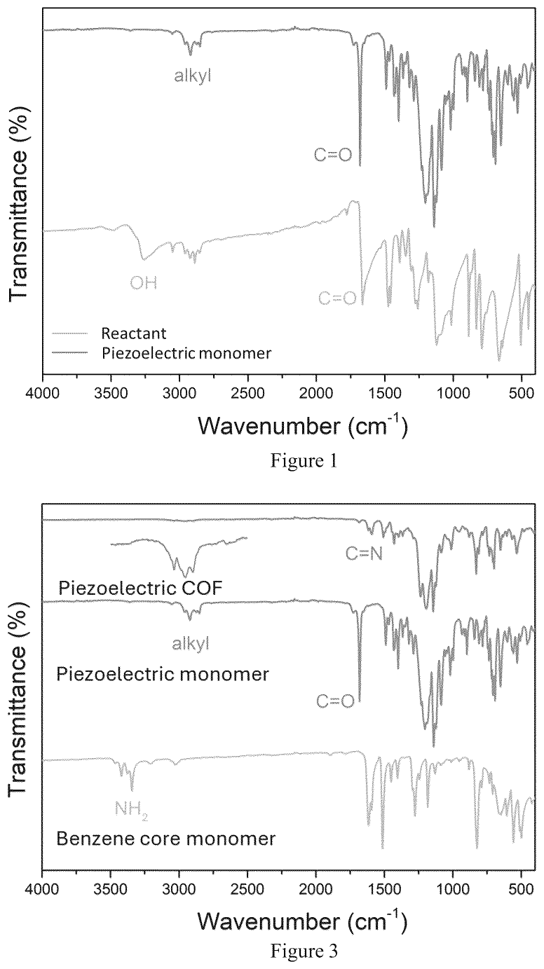

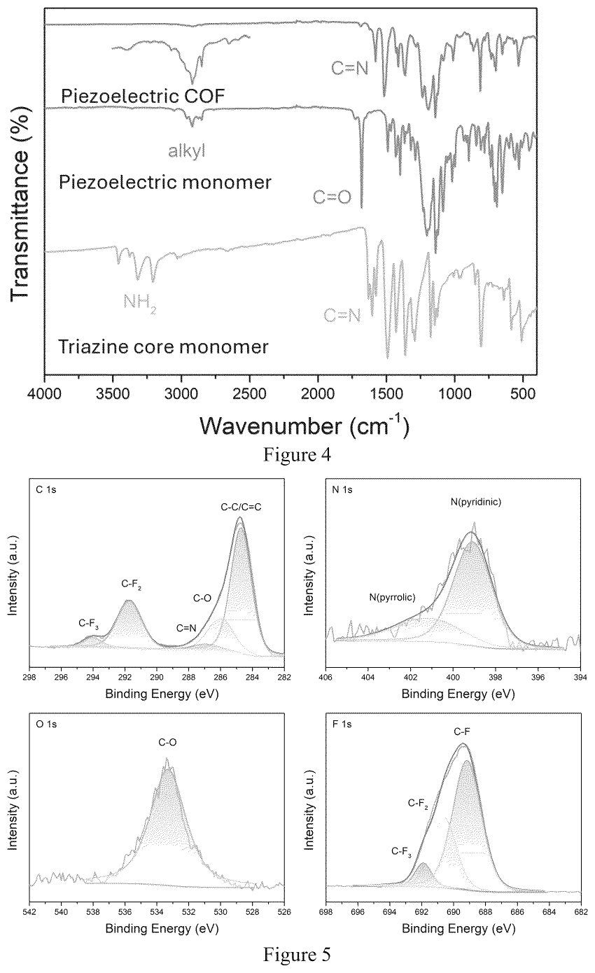

In FIG. 1, the strong peak around 3260 cm⁻¹ assigned to the OH in reactant (13F—OH) was attenuated after reacting with 13F-OTs. The new peak that arose around 2920 cm⁻¹ was assigned to the alkyl signals after substitution of OH, indicating the fluorinated alkyl chain was anchored. In FIGS. 3 and 4, The vanished peaks at 1680 cm⁻¹ (assigned to C═O) and in the range of 3200-3470 cm⁻¹ (assigned to NH₂), the newly appeared peaks at 1620 cm⁻¹ (assigned to C═N), and the unchanged peak at 1200 cm⁻¹ (assigned to C—F) indicated the successful polycondensation between 13F—CHO and TAPB or TAPT.

b. NMR

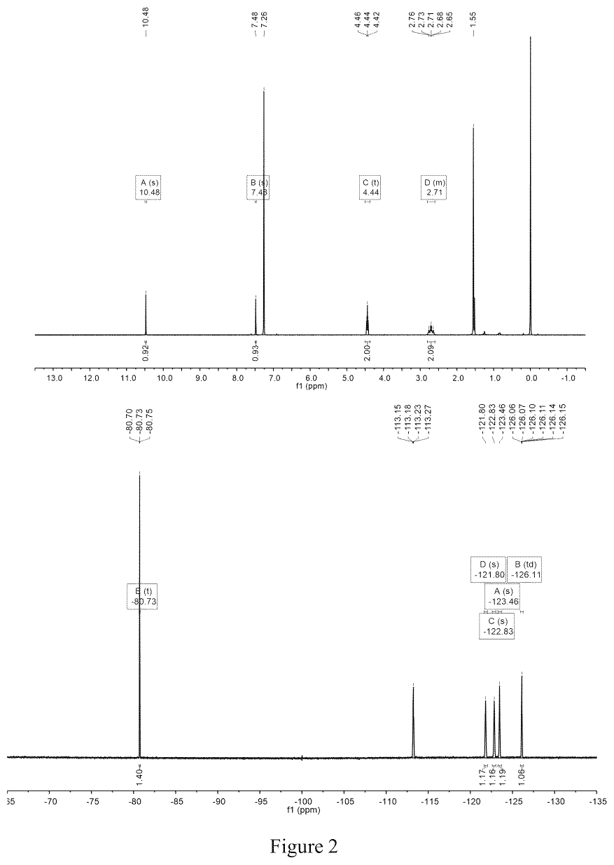

In FIG. 2, the signals from those hydrogens in compounds 13F—CHO were well defined. Moreover, with the help of ¹⁹F-NMR, the fluorine in compounds 13F—CHO were identified as well.

c. XPS

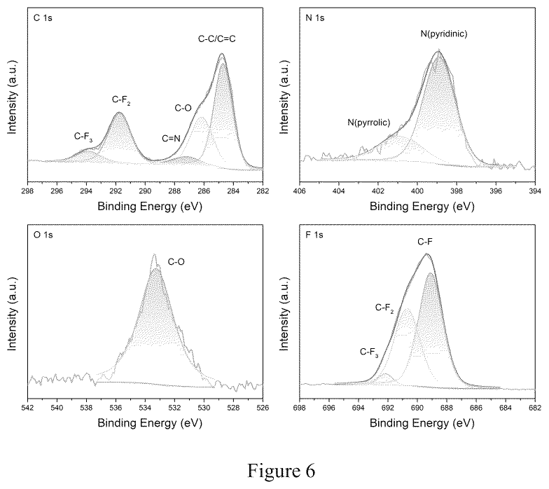

In FIGS. 5 and 6, the pronounced signals in X-ray photoelectron spectroscopy (XPS) spectra indicate the elemental and bonding information of corresponding piezoelectric COFs. The peaks at 399.0 eV in N1s spectra are attributed to the C═N bond while the peaks at 291.7 eV and 293.9 eV in C1s spectra are assigned to fluorinated alkyl groups. Besides, the peaks at 689.1 eV, 690.6 eV and 692.1 eV in F1s spectra are also assigned to fluorinated alkyl groups.

d. PXRD

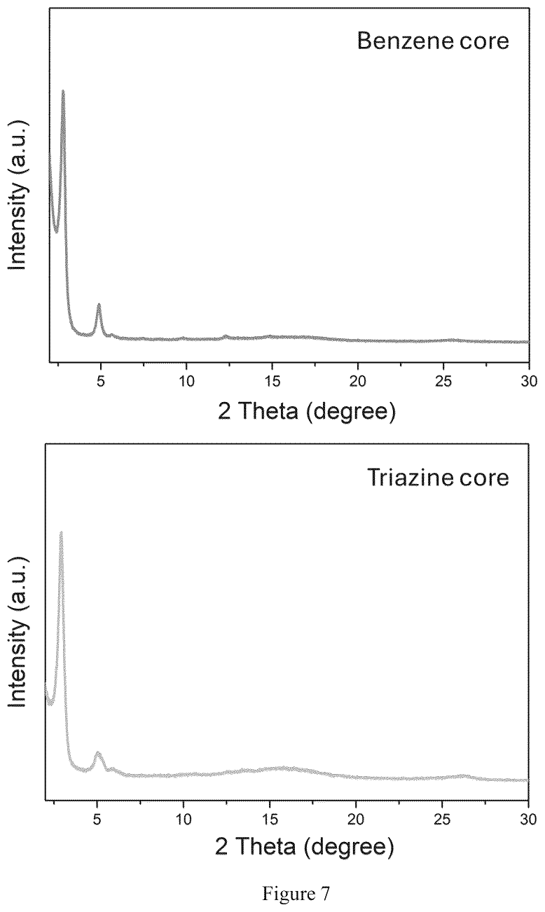

In FIG. 7, the sharp peak in powder X-ray diffraction patterns confirm the ordered structure with high crystallinity of piezoelectric COFs. The diffraction peaks at 2.780 for benzene core COF and 2.91° for triazine core COF belong to the (100) facet, while the diffraction signal at 4.89° for benzene core COF and 5.10° for triazine core COF are ascribed to the (110) facet.

e. HRTEM

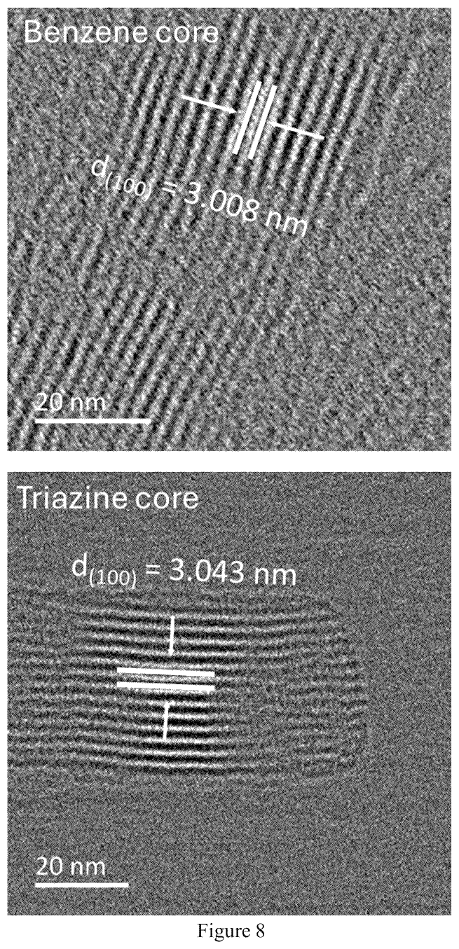

In FIG. 8, high resolution transmission electron microscope (HRTEM) images demonstrate the ordered structure of piezoelectric COFs. The regular lattice fringes with spacing of 3.01 nm for benzene core COF and 3.04 nm for triazine core COF were ascribed to the (100) plane. Moreover, the lattice spacing of two COFs were consistent with the pore diameters of benzene core COF (27.98 Å) and triazine core COF (28.33 Å) in the simulated COF structures, indicating that the grid stacking along the c axis led to the distinct one-dimensional open channels with a uniform diameter.

### Example 4: Piezoelectric Properties

a. PFM

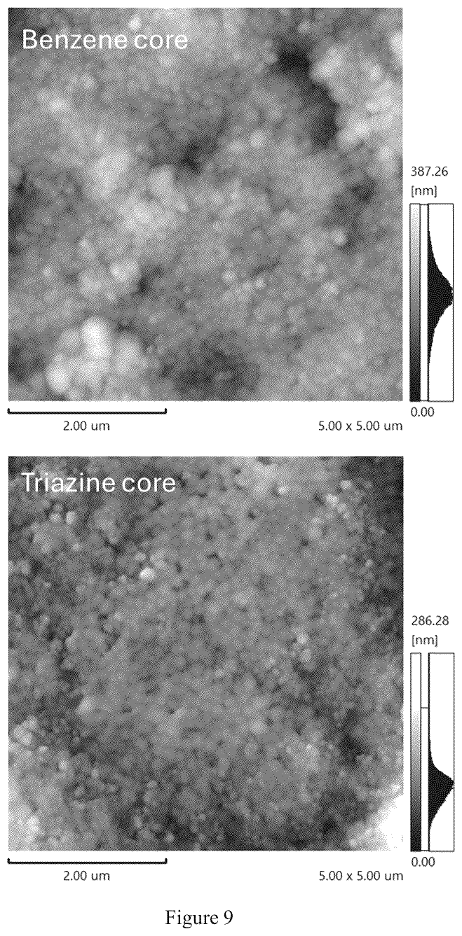

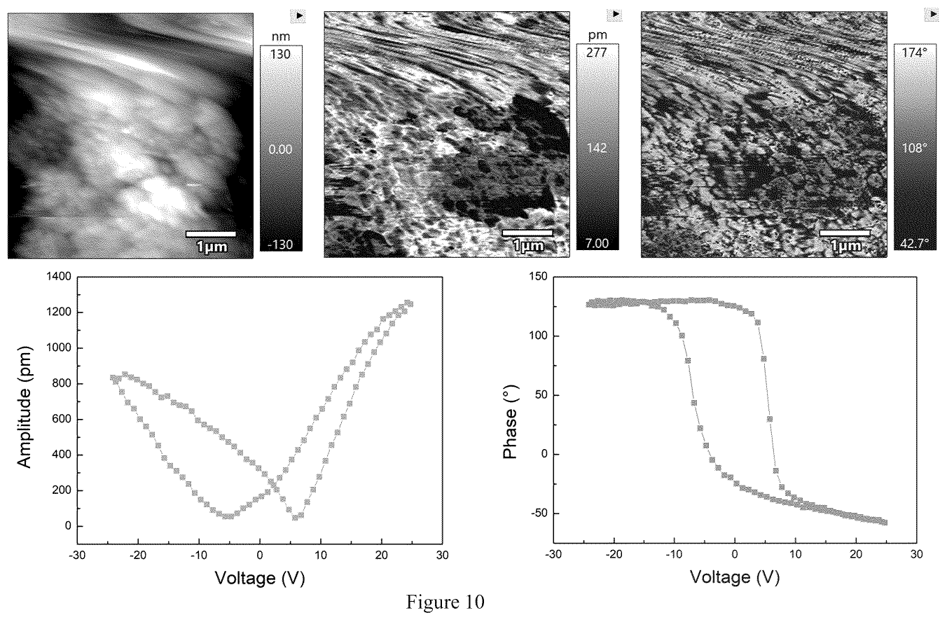

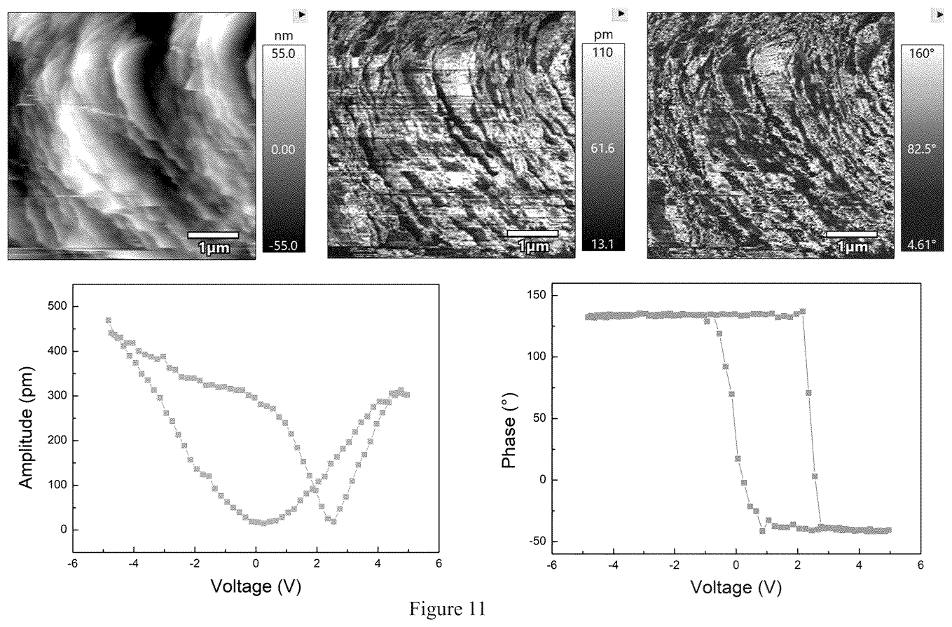

In FIG. 9, atomic force microscopy (AFM) test was carried out first in a 5×5 μm² range to investigate the morphology. In FIGS. 10 and 11, the topographic images of benzene core COF and triazine core COF were acquired in the same range using piezoelectric response force microscopy (PFM). The vertical PFM amplitude and corresponding phase diagrams of both COFs were polled by the tip voltage of ±25 V from the same region. The dramatic differences in amplitude and corresponding phases with the change in bias indicate significant changes of intensity and polarization direction in the domain structures. In addition, nanoscale hysteresis loops underneath the PFM tip were further carried out to verify the ferroelectric domains and switchable polarization. The butterfly shaped amplitude hysteresis loops and corresponding phase hysteresis loops dependent on bias voltage confirmed that the polarization of both COFs could be switched by electric field. Such a switchable nature of the domain structures provides robust evidence for the occurrence of piezoelectricity in two COFs.

b. KPFM

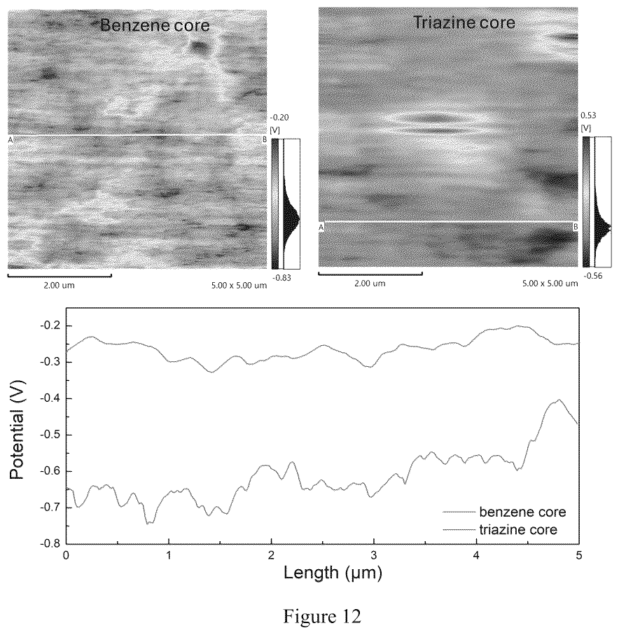

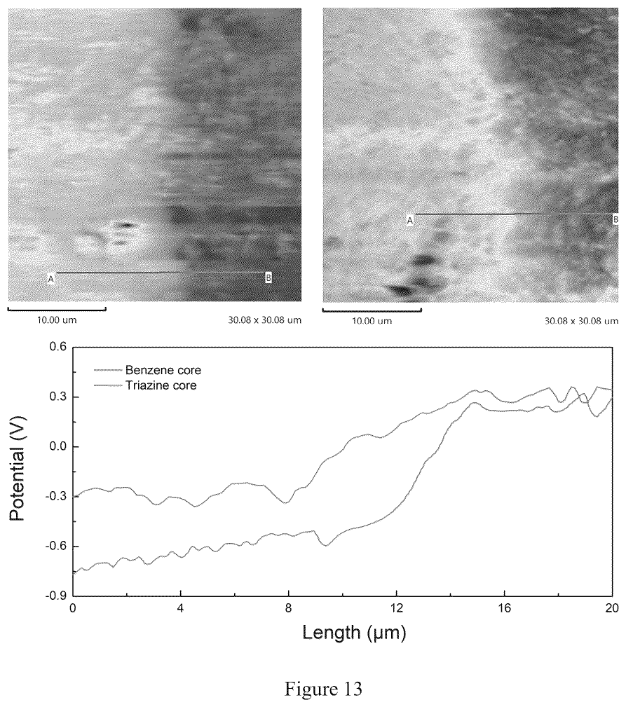

In FIGS. 12 and 13, the Kelvin probe force microscope (KPFM) was employed to measure surface potential and further provide powerful evidence for piezoelectric property. Under the stress of the KPFM probe tip, an inward built-in electric field formed inside both COFs could migrate electrons outward to the surface of COFs. Therefore, a negative surface voltage of about 0.6 V for benzene core COF and 0.3 V for triazine core COF was generated. The negative surface potential was uniformly distributed on the surface of both COFs, which was more obvious compared to the aluminum (Al) substrate.

c. Piezoelectric Coefficient (d₃₃)

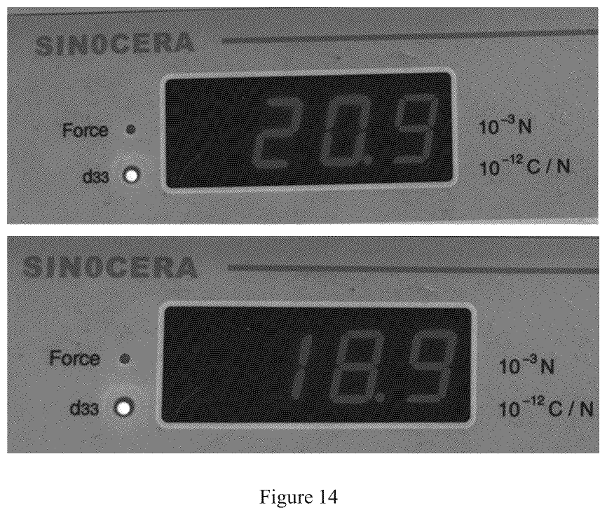

In FIG. 14, the direct piezoelectric effect of both COFs was determined by a quasi-static method. High piezoelectric coefficient d₃₃ was obtained for benzene core COF (20.9 pC/N) and triazine core COF (18.9 pC/N), testifying the high piezoelectric response of both COFs.

### Example 5: Piezoelectric Nanogenerator (PENG)

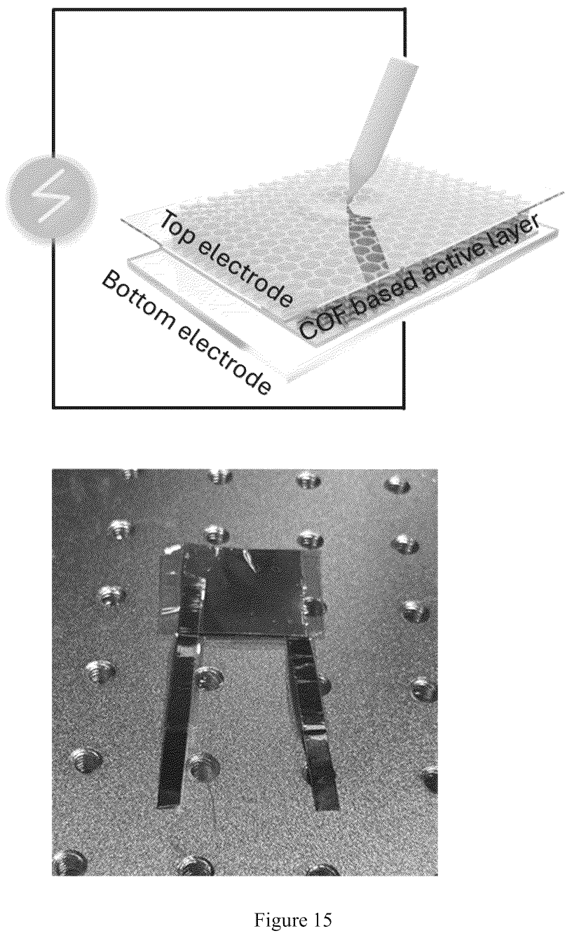

Benzene core COF and triazine core COF were employed as the active layer to fabricate the PENG with Al top and bottom electrodes. The schematic illustration and photos of the PENG were shown in FIG. 15.

a. Output Open Circuit Voltage (V_{oc})

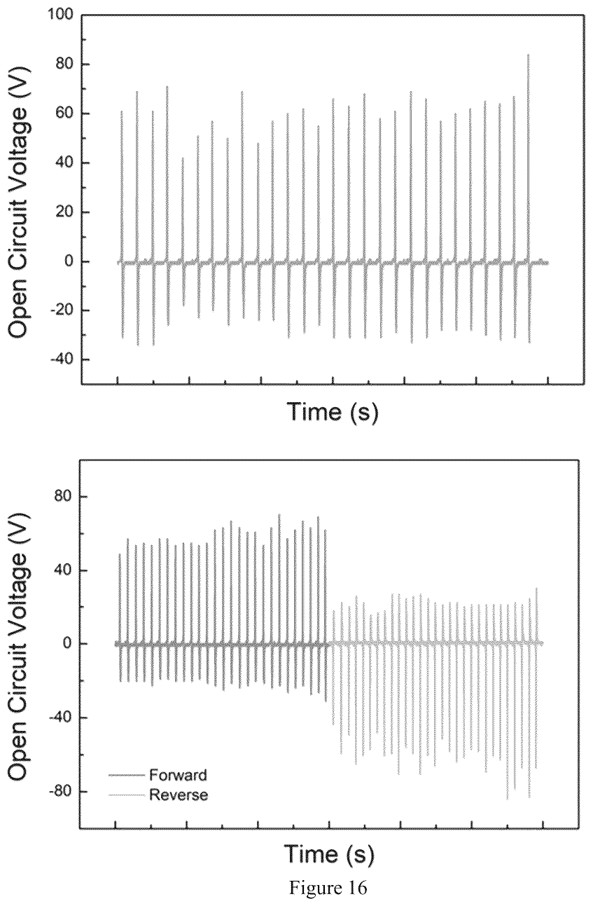

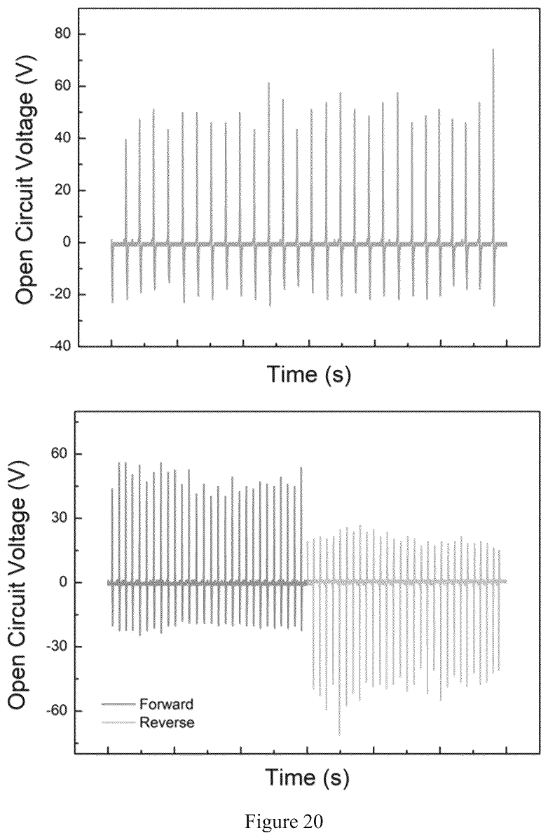

In FIG. 16, the open circuit voltage (V_{oc}) for PENG based on benzene core COF is quite high (60 V). The positive voltage originated from the dipole alignment and net spontaneous polarization upon mechanical contact, where dipoles came from substituted F-containing alkyl chains. The dipole alignment and net spontaneous polarization were further confirmed by the polarity switching test, where the voltage peaks could be positive for forward connection and negative for reverse connection. This result confirms that the origin of electrical output is only from the COFs, further evidencing the dipole alignment in both COFs. The same phenomenon could be found for triazine core COF as shown in FIG. 20, where the V_{oc} is high up to 50 V. The exceptional performance of both COFs-based PENGs, especially for the output open circuit voltage, is currently the highest among all reported organic piezoelectric materials.

b. Output Short Circuit Current (I_{sc}) and Instantaneous Power (P)

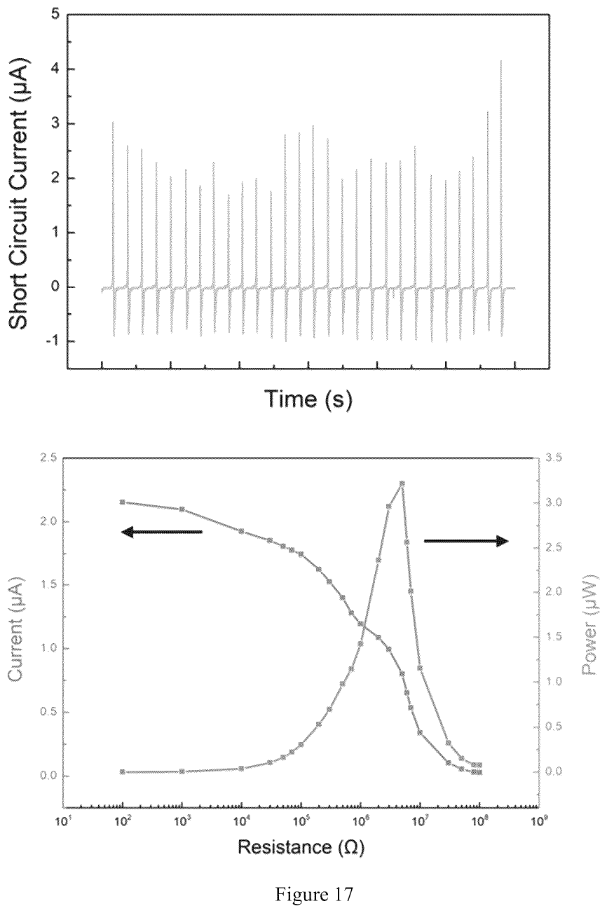

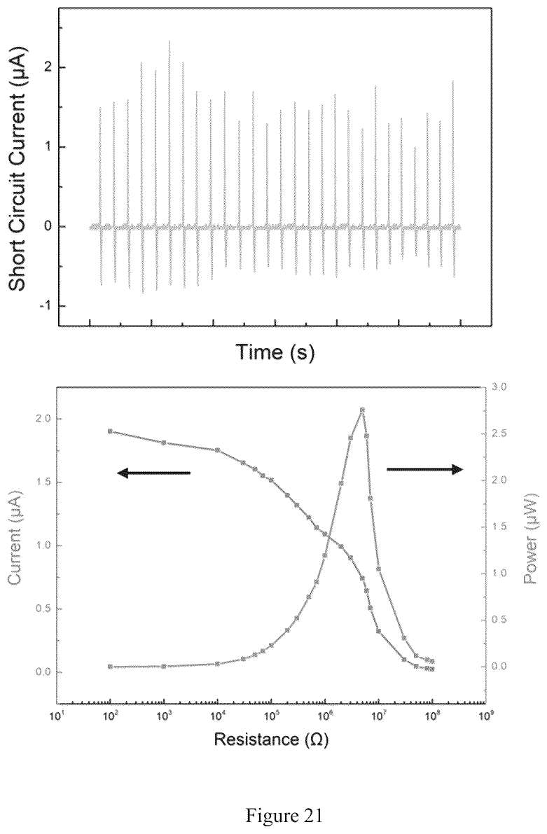

In FIG. 17, the short circuit current (I_{sc}) of PENGs based on benzene core COF is 2.4 μA. The output current gradually raised with the increase of external loading resistances. The currents of COFs-based PENGs were changed upon various resistance, indicating the current originated from both COFs rather than the input power source. Furthermore, the instantaneous power (P) of PENGs based on benzene core COF was calculated by varying the loading resistances from 100Ω to 100 MΩ. The instantaneous power of the benzene core COF based PENG device reached 3.3 μW at the 5 MΩ load resistance. In FIG. 21, the I_{sc} for triazine core COF is 1.5 μA while the instantaneous power of its PENG device reached 2.8 μW at the 5 MΩ load resistance. The exceptional power output demonstrates the proficiency in harvesting sustainable energy from mechanical impacts. Hence, they will be well-suited for self-powered electronic devices, eliminating the necessity for any external batteries.

c. Stability

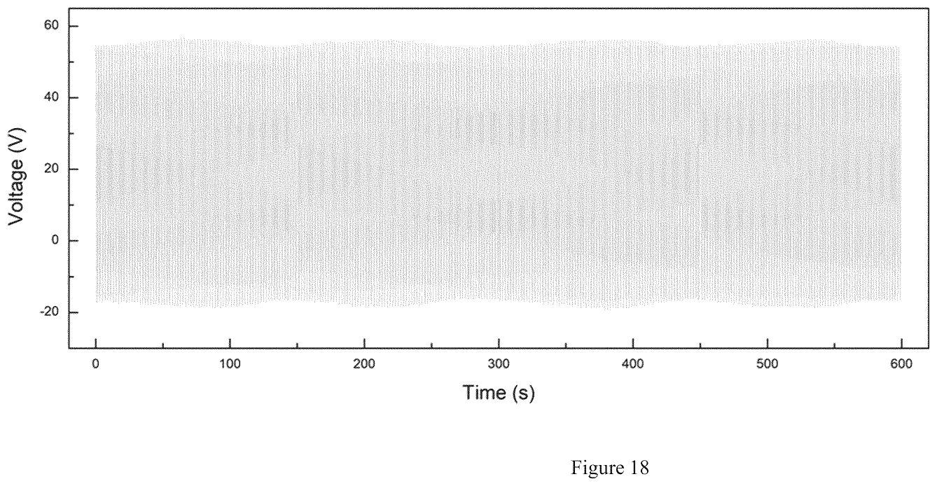

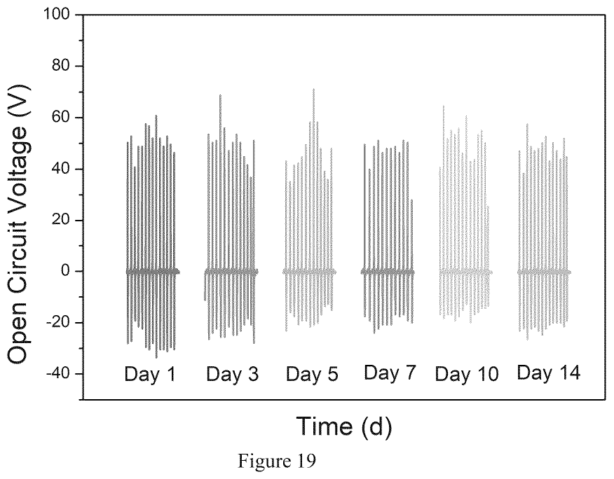

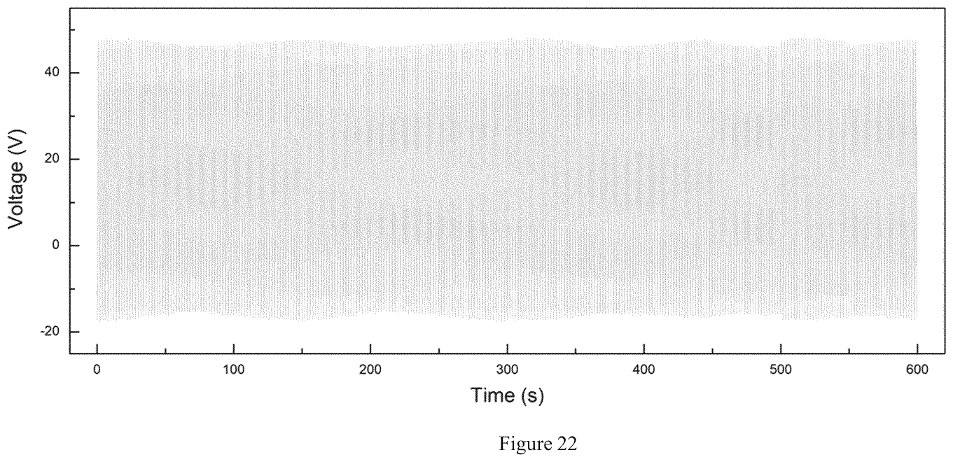

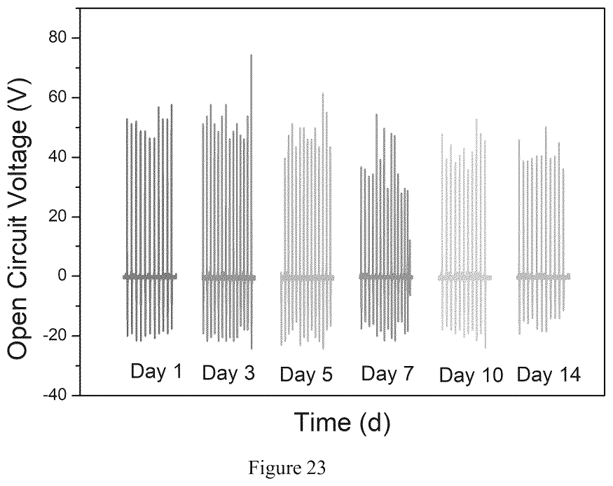

Stability is a pivotal parameter for practical applications. Superior long-term durability (over 600s) of the PENG based on two piezoelectric COFs were revealed under continuous force without any noticeable degradations of piezoelectric output voltage (as shown in FIGS. 18 and 22). Additionally, two COFs based PENG could be operated for even longer duration (up to 14 days) without obvious changes in output voltages, indicating the superior stability for practical application (as shown in FIGS. 19 and 23).

d. Practical Performance

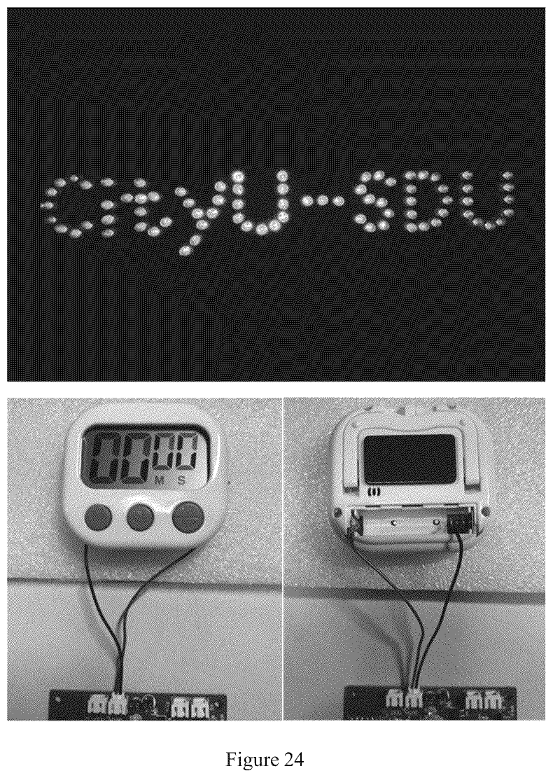

In FIG. 24, to verify the practical performance, piezoelectric COF based PENG was employed to directly power up an array of 76 light emitting diodes (LEDs) with the word pattern of “CityU-SDU” and a LED display timer by using the full-wave bridge rectifier circuit and capacitor.

According to another aspect of the invention, there is provided two two-dimensional (2D) covalent organic frameworks (COFs, CityU-13 and CityU-14), functionalized with fluorinated alkyl chains for PENGs. The piezoelectricity of both COFs was evidenced by switchable polarization, characteristic butterfly amplitude loops, phase hysteresis loops, conspicuous surface potentials and high piezoelectric coefficient value (d₃₃). The PENGs fabricated with COFs displayed highest output open circuit voltages (60 V for CityU-13 and 50 V for CityU-14) and delivered satisfactory short circuit current with an excellent stability of over 600 seconds. The superior open circuit voltages of CityU-13 and CityU-14 rank in top 1 and 2 among all reported organic materials-based PENGs.

In particular, two imine-linked COFs (named as CityU-13 and CityU-14) with 3,3,4,4,5,5,6,6,7,7,8,8,8-tridecafluorooctyl side chains are presented. The fluorinated polar chains were anchored to trigger the asymmetry and intrinsic polarizability for non-negligible piezoelectric properties. The piezoelectric properties were demonstrated by dipole moment, piezoelectric force microscopy (PFM), kelvin probe force microscopy (KPFM) and piezoelectric coefficient (d₃₃). Applying them in PENGs, both COFs exhibit superb output open circuit voltage (V_{oc}, 60 V for CityU-13; 50 V for CityU-14), short circuit current (I_{sc}, 2.4 μA for CityU-13; 1.5 μA for CityU-14), instantaneous power (P, 3.3 μW for CityU-13; 2.8 W for CityU-14) and long stability (over 600 s) under an applied force without poling conditions. This marks the inaugural instance where a fluorinated alkyl motif has been incorporated into COFs for piezoelectric applications, thereby establishing the groundwork for the development of functionalized COFs and broadening the horizon of low-dimensional piezoelectric materials towards PENGs and self-powered systems.

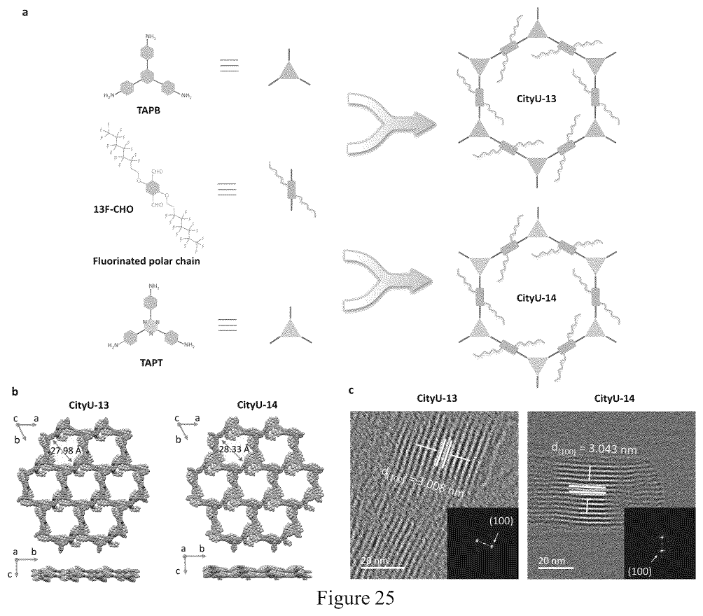

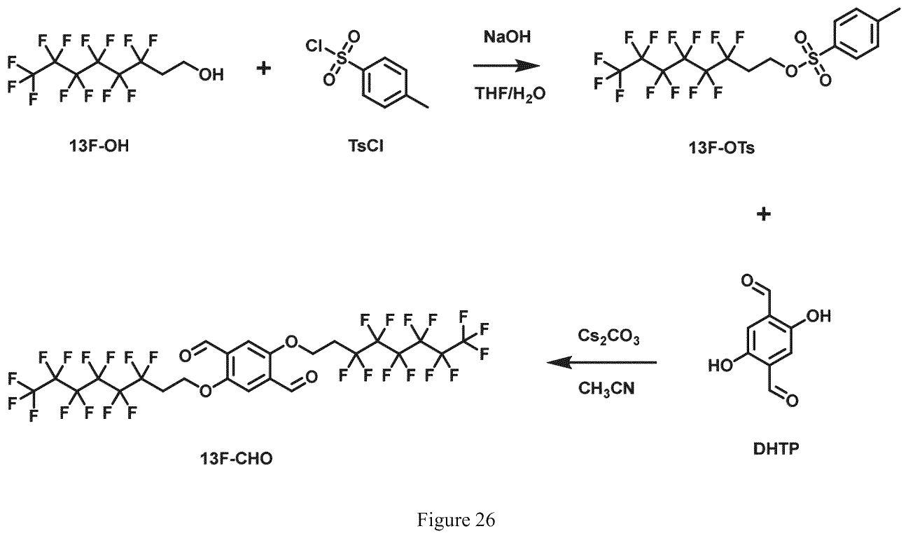

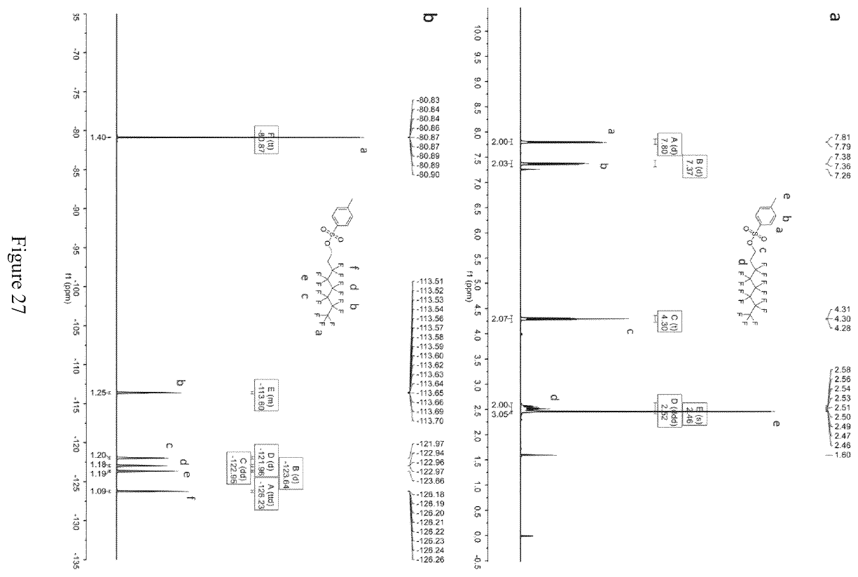

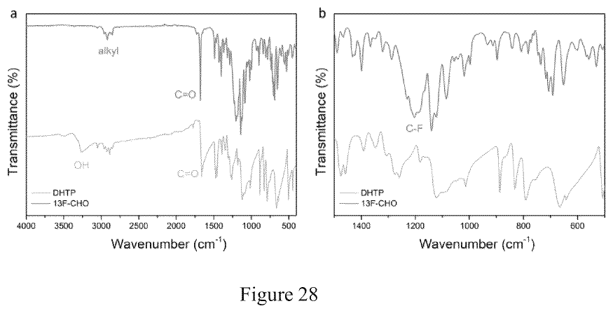

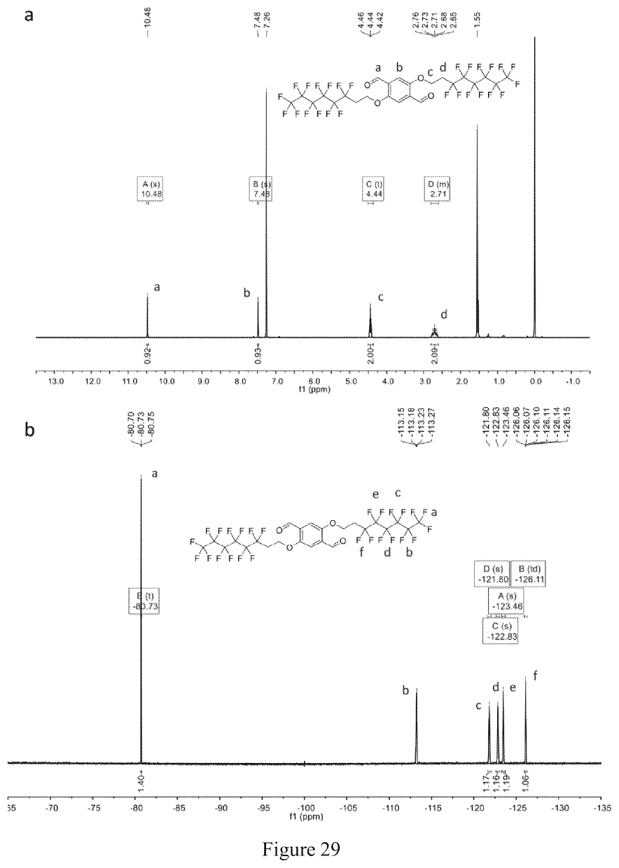

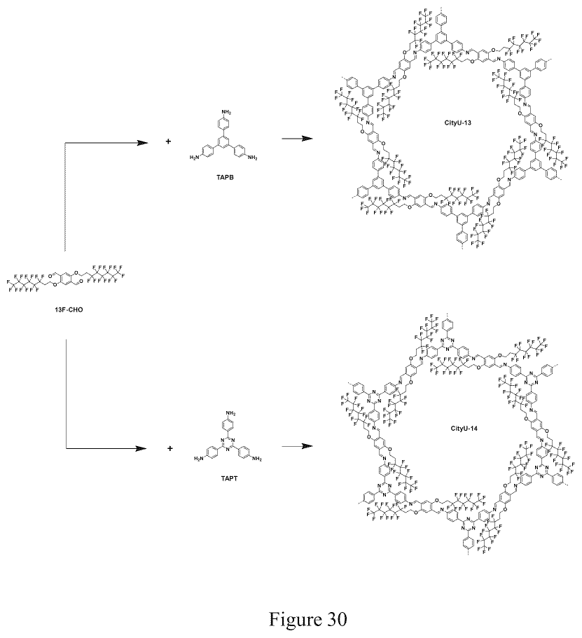

The synthetic routes of CityU-13 and CityU-14 were illustrated in FIG. 25a, where the COFs precursor 2,5-bis((3,3,4,4,5,5,6,6,7,7,8,8,8-tridecafluorooctyl)oxy)terephthalaldehyde (13F—CHO) was synthesized using 3,3,4,4,5,5,6,6,7,7,8,8,8-tridecafluorooctan-1-ol (13F—OH) as a starting agent (FIG. 26). The structure of 13F—CHO was confirmed by nuclear magnetic resonance (NMR) and Fourier transform infrared (FTIR) spectra (FIGS. 27-29). CityU-13 and CityU-14 were obtained by reacting 13F—CHO with 1,3,5-Tris(4-aminophenyl) benzene (TAPB) or tris(4-aminophenyl) triazine (TAPT) in a mixed solvent of superdry 1,2-dichlorobenzene (DCB) and n-butyl alcohol (n-BuOH) under the catalysis of acetic acid (HOAc) at 120° C. for 72 hours (FIG. 25a and FIG. 30).

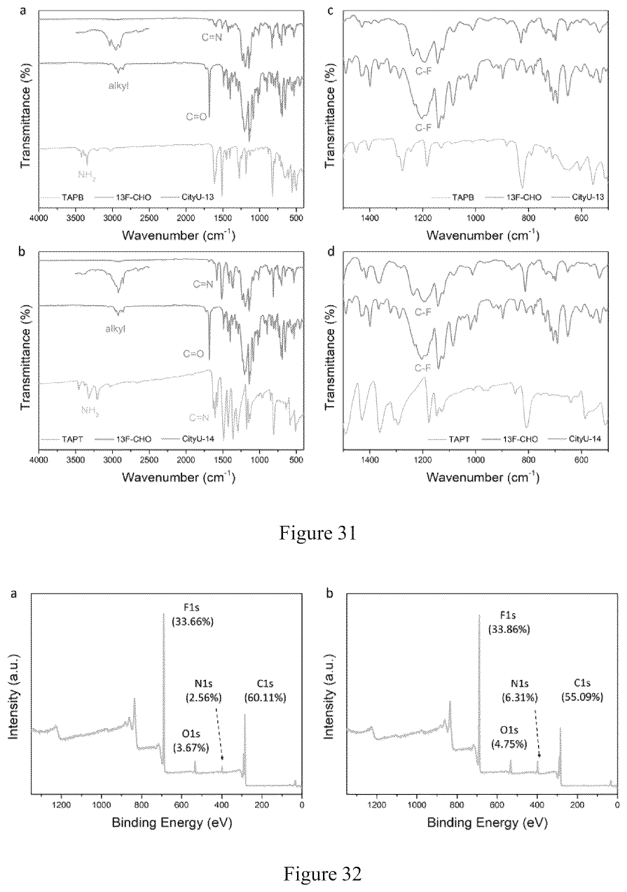

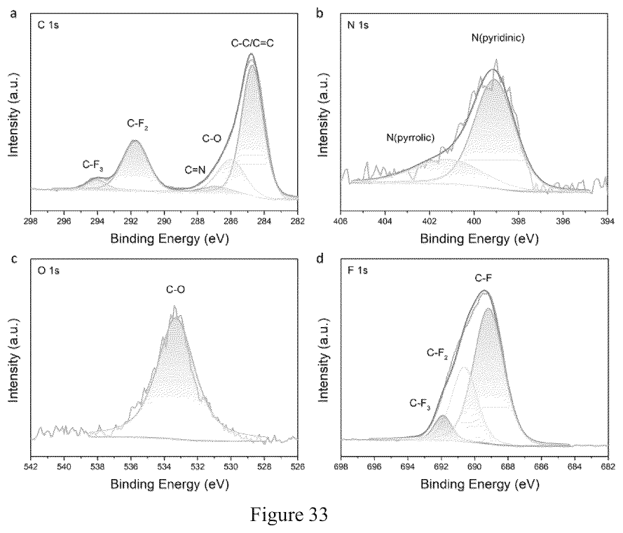

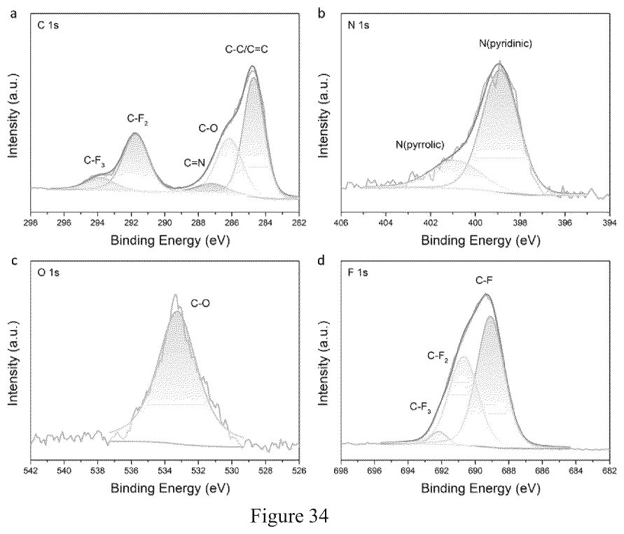

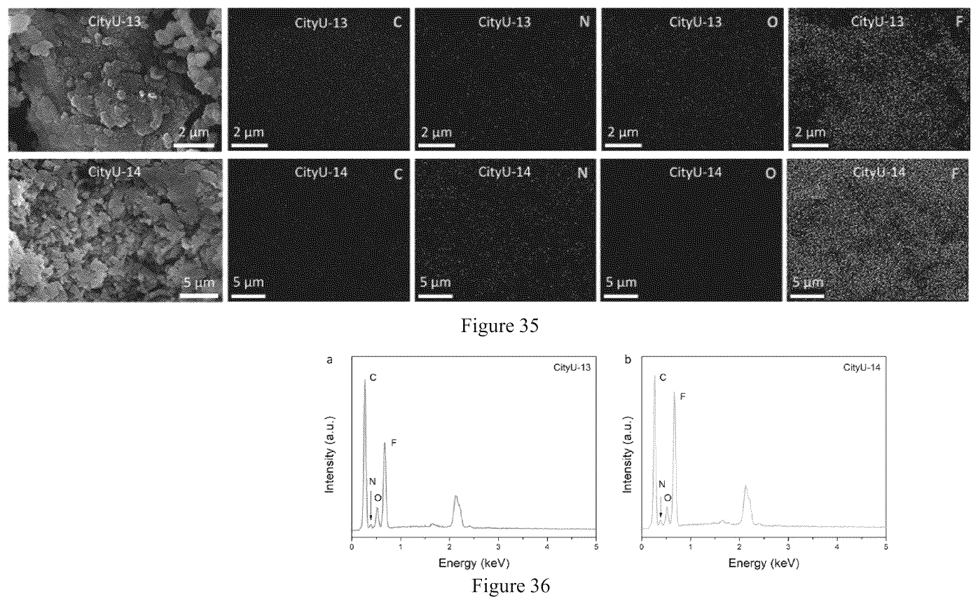

The structures of CityU-13 and CityU-14 were firstly characterized by FTIR spectra. The vanished peaks at 1680 cm⁻¹ (C═O) and in the range of 3200-3470 cm⁻¹ (NH₂),^{[10]} the newly appeared peaks at 1620 cm⁻¹ (C═N),^{[11]} and the unchanged peak at 1200 cm⁻¹ (C—F) in the FTIR spectra of both COFs (FIG. 31) indicated the successful polycondensation between 13F—CHO and TAPB/TAPT.^{[12]} The C═N bond (399.0 eV in N1s spectra) and fluorinated alkyl groups (291.7 eV and 293.9 eV in C1s spectra; 689.1 eV, 690.6 eV and 692.1 eV in Fis spectra) were further confirmed by X-ray photoelectron spectroscopy (XPS) spectra (FIG. 32-34).^{[13]} Moreover, the existence of several elements (C, N, F, O) were further proven by scanning electron microscopy (SEM) with elemental mapping patterns and energy-dispersive X-ray spectroscopy (EDS), where both COFs exhibited conspicuous and uniformly distributed fluorine signals (FIGS. 35 and 36).

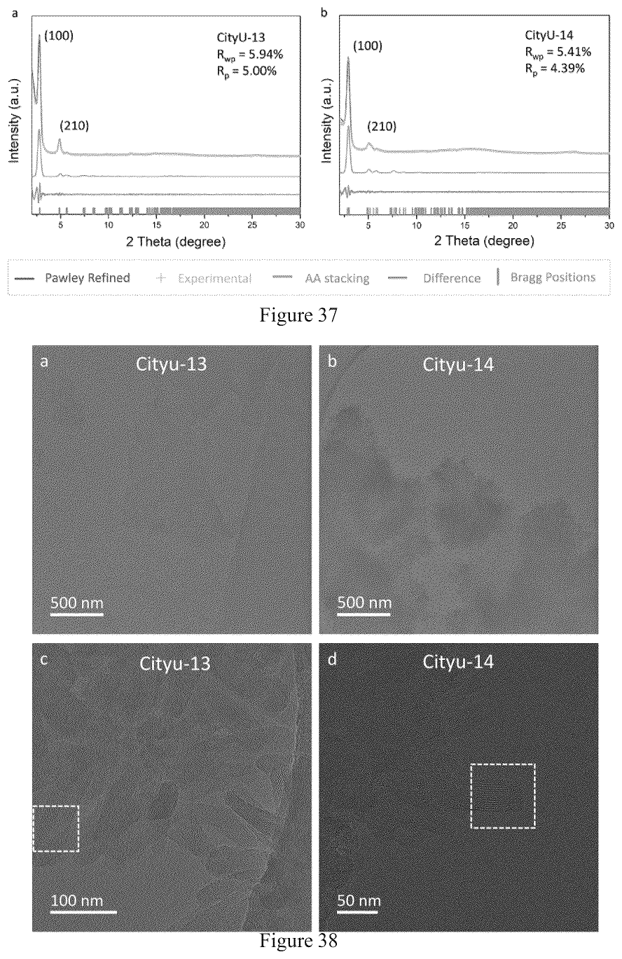

The powder X-ray diffraction (PXRD) patterns of both COFs displayed several sharp peaks, indicating their high crystallinity. The diffraction peaks at 2.78° for CityU-13 and 2.91° for CityU-14 belong to the (100) facet, while the diffraction signal at 4.890 for CityU-13 and 5.10° for CityU-14 are ascribed to the (110) facet (FIG. 37). The structures of CityU-13 and CityU-14 were also studied through structural simulation with geometrical energy minimizations (Materials Studio 2020, BIOVIA).^{[14]} Both COFs displayed layer structures with an AA-stacking model (FIG. 25b). The simulated diffraction profiles showed satisfied consistency with the experimental results. After Pawley refinement, the unit cell parameters of both COFs were iteratively optimized with the convergence of R_{wp} and Rₚ. The R_{wp} and Rₚ for CityU-13 are 5.94% and 5.00% (FIG. 37a) with the unit cell parameters of a=35.2503 Å, b=36.0951 Å, c=5.6874 Å, α=88.3437°, β=88.7346°, γ=63.8707° (Table S1). The R_{wp} and Rₚ for CityU-14 are 5.41% and 4.39% (FIG. 37b) with the unit cell parameters of a=36.1192 Å, b=36.3344 Å, c=5.5680 Å, α=88.1417°, β=92.1170°, γ=61.2965° (Table S2). The satisfactory converged values of Rₚ and R_{wp} imply the high consistency between experimental and Pawley refined profiles.

TABLE S1
Fractional atomic coordinates for the unit cell of CityU-13.
CityU-13: space group P1
a = 35.2503 Å, b = 36.0951 Å, c = 5.6874 Å
α = 88.3437°, β = 88.7346°, γ = 63.8707°

| | | | | | U_iso | | |
|---|---|---|---|---|---|---|---|
| | | | | | or | | occu- |
| label | atom | fract_x | fract_y | fract_z | equiv | adp_type | pancy |
| C1 | C | 0.33999 | 0.21936 | 0.0809 | 0 | Uiso | 1 |
| C2 | C | 0.37852 | 0.2019 | 0.95509 | 0 | Uiso | 1 |
| C3 | C | 0.40058 | 0.15912 | 0.9338 | 0 | Uiso | 1 |
| C4 | C | 0.38485 | 0.13277 | 0.03506 | 0 | Uiso | 1 |
| C5 | C | 0.34603 | 0.15018 | 0.15841 | 0 | Uiso | 1 |
| C6 | C | 0.32375 | 0.19299 | 0.17907 | 0 | Uiso | 1 |
| N7 | N | 0.41097 | 0.08892 | 0.02536 | 0 | Uiso | 1 |
| C8 | C | 0.40131 | 0.05923 | 0.0907 | 0 | Uiso | 1 |
| C9 | C | 0.43372 | 0.01549 | 0.08781 | 0 | Uiso | 1 |
| C10 | C | 0.47764 | 0.00442 | 0.07882 | 0 | Uiso | 1 |
| C11 | C | 0.50676 | 0.96316 | 0.06743 | 0 | Uiso | 1 |
| C12 | C | 0.49376 | 0.93167 | 0.07514 | 0 | Uiso | 1 |
| C13 | C | 0.44987 | 0.94223 | 0.08653 | 0 | Uiso | 1 |
| C14 | C | 0.42055 | 0.98385 | 0.09734 | 0 | Uiso | 1 |
| C15 | C | 0.52715 | 0.88879 | 0.06583 | 0 | Uiso | 1 |
| N16 | N | 0.52086 | 0.85826 | 0.15141 | 0 | Uiso | 1 |
| C17 | C | 0.54978 | 0.81537 | 0.13442 | 0 | Uiso | 1 |
| C18 | C | 0.58067 | 0.80045 | 0.95576 | 0 | Uiso | 1 |
| C19 | C | 0.60508 | 0.75799 | 0.93164 | 0 | Uiso | 1 |
| C20 | C | 0.59908 | 0.72973 | 0.08552 | 0 | Uiso | 1 |
| C21 | C | 0.56872 | 0.74489 | 0.26614 | 0 | Uiso | 1 |
| C22 | C | 0.5443 | 0.78734 | 0.28784 | 0 | Uiso | 1 |
| C23 | C | 0.62174 | 0.68486 | 0.04219 | 0 | Uiso | 1 |
| C24 | C | 0.66399 | 0.66744 | 0.96629 | 0 | Uiso | 1 |
| C25 | C | 0.68378 | 0.62682 | 0.88767 | 0 | Uiso | 1 |
| C26 | C | 0.66115 | 0.60313 | 0.88955 | 0 | Uiso | 1 |
| C27 | C | 0.61888 | 0.61978 | 0.96976 | 0 | Uiso | 1 |
| C28 | C | 0.59984 | 0.66045 | 0.05121 | 0 | Uiso | 1 |
| C29 | C | 0.59336 | 0.5967 | 0.93844 | 0 | Uiso | 1 |
| C30 | C | 0.61228 | 0.5533 | 0.95319 | 0 | Uiso | 1 |
| C31 | C | 0.59011 | 0.53183 | 0.88102 | 0 | Uiso | 1 |
| C32 | C | 0.54912 | 0.55248 | 0.79714 | 0 | Uiso | 1 |
| C33 | C | 0.52885 | 0.59565 | 0.7995 | 0 | Uiso | 1 |
| C34 | C | 0.55076 | 0.61767 | 0.86868 | 0 | Uiso | 1 |
| N35 | N | 0.53073 | 0.52819 | 0.69792 | 0 | Uiso | 1 |
| O36 | O | 0.43439 | 0.91239 | 0.09224 | 0 | Uiso | 1 |
| O37 | O | 0.49348 | 0.03364 | 0.07249 | 0 | Uiso | 1 |
| C38 | C | 0.5009 | 0.54047 | 0.54347 | 0 | Uiso | 1 |
| C39 | C | 0.48832 | 0.51059 | 0.43986 | 0 | Uiso | 1 |
| C40 | C | 0.51617 | 0.46786 | 0.42656 | 0 | Uiso | 1 |
| C41 | C | 0.50098 | 0.4406 | 0.35076 | 0 | Uiso | 1 |
| C42 | C | 0.4589 | 0.45476 | 0.28148 | 0 | Uiso | 1 |
| C43 | C | 0.43211 | 0.4978 | 0.27332 | 0 | Uiso | 1 |
| C44 | C | 0.44696 | 0.52509 | 0.35487 | 0 | Uiso | 1 |
| C45 | C | 0.44347 | 0.42385 | 0.23587 | 0 | Uiso | 1 |
| N46 | N | 0.40559 | 0.43076 | 0.30203 | 0 | Uiso | 1 |
| O47 | O | 0.55834 | 0.45086 | 0.50011 | 0 | Uiso | 1 |
| O48 | O | 0.38992 | 0.51431 | 0.20092 | 0 | Uiso | 1 |
| C49 | C | 0.72678 | 0.61049 | 0.78411 | 0 | Uiso | 1 |
| C50 | C | 0.73716 | 0.63471 | 0.62051 | 0 | Uiso | 1 |
| C51 | C | 0.77744 | 0.61914 | 0.5173 | 0 | Uiso | 1 |
| C52 | C | 0.8074 | 0.57915 | 0.57284 | 0 | Uiso | 1 |
| C53 | C | 0.79771 | 0.55542 | 0.74128 | 0 | Uiso | 1 |
| C54 | C | 0.75745 | 0.57065 | 0.8445 | 0 | Uiso | 1 |
| N55 | N | 0.8489 | 0.56431 | 0.47376 | 0 | Uiso | 1 |
| C56 | C | 0.86835 | 0.5289 | 0.37348 | 0 | Uiso | 1 |
| C57 | C | 0.91417 | 0.51108 | 0.32021 | 0 | Uiso | 1 |
| C58 | C | 0.93718 | 0.53485 | 0.31487 | 0 | Uiso | 1 |
| C59 | C | 0.98077 | 0.51564 | 0.27638 | 0 | Uiso | 1 |
| C60 | C | 0.00266 | 0.47278 | 0.24986 | 0 | Uiso | 1 |
| C61 | C | 0.97977 | 0.44886 | 0.25632 | 0 | Uiso | 1 |
| C62 | C | 0.93594 | 0.46827 | 0.28692 | 0 | Uiso | 1 |
| C63 | C | 0.04924 | 0.45434 | 0.22517 | 0 | Uiso | 1 |
| N64 | N | 0.07102 | 0.41584 | 0.17283 | 0 | Uiso | 1 |
| C65 | C | 0.11626 | 0.39467 | 0.16889 | 0 | Uiso | 1 |
| C66 | C | 0.14067 | 0.40375 | 0.32816 | 0 | Uiso | 1 |
| C67 | C | 0.18459 | 0.38121 | 0.32778 | 0 | Uiso | 1 |
| C68 | C | 0.20493 | 0.34977 | 0.16417 | 0 | Uiso | 1 |
| C69 | C | 0.18031 | 0.34011 | 0.00915 | 0 | Uiso | 1 |
| C70 | C | 0.13624 | 0.36189 | 0.01643 | 0 | Uiso | 1 |
| C71 | C | 0.25162 | 0.32796 | 0.15358 | 0 | Uiso | 1 |
| C72 | C | 0.27515 | 0.35023 | 0.17749 | 0 | Uiso | 1 |
| C73 | C | 0.31919 | 0.33058 | 0.17966 | 0 | Uiso | 1 |
| C74 | C | 0.34023 | 0.28776 | 0.14881 | 0 | Uiso | 1 |
| C75 | C | 0.31738 | 0.26474 | 0.11469 | 0 | Uiso | 1 |
| C76 | C | 0.27285 | 0.28524 | 0.11938 | 0 | Uiso | 1 |
| C77 | C | 0.34259 | 0.35535 | 0.214 | 0 | Uiso | 1 |
| C78 | C | 0.32847 | 0.38673 | 0.38111 | 0 | Uiso | 1 |
| C79 | C | 0.34957 | 0.41119 | 0.40556 | 0 | Uiso | 1 |
| C80 | C | 0.38565 | 0.40412 | 0.27141 | 0 | Uiso | 1 |
| C81 | C | 0.39898 | 0.37382 | 0.09851 | 0 | Uiso | 1 |
| C82 | C | 0.37754 | 0.34962 | 0.07039 | 0 | Uiso | 1 |
| C83 | C | 0.43754 | 0.89259 | 0.87297 | 0 | Uiso | 1 |
| C84 | C | 0.42755 | 0.85535 | 0.91275 | 0 | Uiso | 1 |
| C85 | C | 0.45049 | 0.8222 | 0.73023 | 0 | Uiso | 1 |
| C86 | C | 0.43138 | 0.79045 | 0.70905 | 0 | Uiso | 1 |
| C87 | C | 0.46112 | 0.75506 | 0.54598 | 0 | Uiso | 1 |
| C88 | C | 0.43995 | 0.72625 | 0.5014 | 0 | Uiso | 1 |
| C89 | C | 0.46956 | 0.69018 | 0.34647 | 0 | Uiso | 1 |
| C90 | C | 0.44479 | 0.66399 | 0.2967 | 0 | Uiso | 1 |
| C91 | C | 0.4948 | 0.05025 | 0.29573 | 0 | Uiso | 1 |
| C92 | C | 0.50508 | 0.08689 | 0.25013 | 0 | Uiso | 1 |
| C93 | C | 0.48256 | 0.12206 | 0.42048 | 0 | Uiso | 1 |
| C94 | C | 0.49313 | 0.15917 | 0.35438 | 0 | Uiso | 1 |
| C95 | C | 0.46128 | 0.19889 | 0.47575 | 0 | Uiso | 1 |
| C96 | C | 0.47012 | 0.23572 | 0.38772 | 0 | Uiso | 1 |
| C97 | C | 0.45201 | 0.27108 | 0.56804 | 0 | Uiso | 1 |
| C98 | C | 0.48964 | 0.26878 | 0.71963 | 0 | Uiso | 1 |
| C99 | C | 0.38437 | 0.5118 | 0.95416 | 0 | Uiso | 1 |
| C100 | C | 0.33709 | 0.52747 | 0.90096 | 0 | Uiso | 1 |
| C101 | C | 0.2736 | 0.51094 | 0.88384 | 0 | Uiso | 1 |
| C102 | C | 0.25942 | 0.47632 | 0.93748 | 0 | Uiso | 1 |
| C103 | C | 0.21289 | 0.49235 | 0.85451 | 0 | Uiso | 1 |
| C104 | C | 0.19807 | 0.45856 | 0.90424 | 0 | Uiso | 1 |
| C105 | C | 0.15068 | 0.4769 | 0.82451 | 0 | Uiso | 1 |
| C106 | C | 0.58618 | 0.45987 | 0.34787 | 0 | Uiso | 1 |
| C107 | C | 0.63064 | 0.43915 | 0.45296 | 0 | Uiso | 1 |
| C108 | C | 0.70358 | 0.43795 | 0.47606 | 0 | Uiso | 1 |
| C109 | C | 0.72558 | 0.46694 | 0.45993 | 0 | Uiso | 1 |
| C110 | C | 0.77366 | 0.43914 | 0.4896 | 0 | Uiso | 1 |
| C111 | C | 0.79542 | 0.46602 | 0.55367 | 0 | Uiso | 1 |
| C112 | C | 0.84453 | 0.43776 | 0.53278 | 0 | Uiso | 1 |
| F113 | F | 0.47497 | 0.29709 | 0.90127 | 0 | Uiso | 1 |
| F114 | F | 0.51643 | 0.28041 | 0.58622 | 0 | Uiso | 1 |
| F115 | F | 0.51362 | 0.22982 | 0.8149 | 0 | Uiso | 1 |
| F116 | F | 0.41769 | 0.27077 | 0.70115 | 0 | Uiso | 1 |
| F117 | F | 0.43356 | 0.31009 | 0.45374 | 0 | Uiso | 1 |
| F118 | F | 0.51283 | 0.2243 | 0.34219 | 0 | Uiso | 1 |
| F119 | F | 0.45001 | 0.25058 | 0.17056 | 0 | Uiso | 1 |
| F120 | F | 0.46468 | 0.19358 | 0.71765 | 0 | Uiso | 1 |
| F121 | F | 0.41873 | 0.20797 | 0.43075 | 0 | Uiso | 1 |
| F122 | F | 0.49202 | 0.16501 | 0.11098 | 0 | Uiso | 1 |
| F123 | F | 0.53497 | 0.14838 | 0.41832 | 0 | Uiso | 1 |
| F124 | F | 0.4958 | 0.10572 | 0.64402 | 0 | Uiso | 1 |
| F125 | F | 0.43965 | 0.13199 | 0.41457 | 0 | Uiso | 1 |
| F126 | F | 0.46972 | 0.62863 | 0.1699 | 0 | Uiso | 1 |
| F127 | F | 0.40831 | 0.68547 | 0.16915 | 0 | Uiso | 1 |
| F128 | F | 0.43399 | 0.64876 | 0.50184 | 0 | Uiso | 1 |
| F129 | F | 0.48199 | 0.70344 | 0.13638 | 0 | Uiso | 1 |
| F130 | F | 0.50849 | 0.66579 | 0.45959 | 0 | Uiso | 1 |
| F131 | F | 0.4322 | 0.71058 | 0.71503 | 0 | Uiso | 1 |
| F132 | F | 0.40096 | 0.74797 | 0.39322 | 0 | Uiso | 1 |
| F133 | F | 0.46879 | 0.77134 | 0.33309 | 0 | Uiso | 1 |
| F134 | F | 0.50132 | 0.73279 | 0.64625 | 0 | Uiso | 1 |
| F135 | F | 0.42663 | 0.77586 | 0.93251 | 0 | Uiso | 1 |
| F136 | F | 0.39046 | 0.8115 | 0.61881 | 0 | Uiso | 1 |
| F137 | F | 0.44871 | 0.84272 | 0.51643 | 0 | Uiso | 1 |
| F138 | F | 0.4929 | 0.8039 | 0.79299 | 0 | Uiso | 1 |
| O139 | O | 0.91727 | 0.5779 | 0.31886 | 0 | Uiso | 1 |
| O140 | O | 0.99998 | 0.40598 | 0.22976 | 0 | Uiso | 1 |
| C141 | C | 0.90979 | 0.59568 | 0.54657 | 0 | Uiso | 1 |
| C142 | C | 0.94407 | 0.60913 | 0.60241 | 0 | Uiso | 1 |
| C143 | C | 0.9296 | 0.64252 | 0.7912 | 0 | Uiso | 1 |
| C144 | C | 0.96664 | 0.64328 | 0.9361 | 0 | Uiso | 1 |
| C145 | C | 0.99635 | 0.65337 | 0.77003 | 0 | Uiso | 1 |
| C146 | C | 0.02942 | 0.65959 | 0.91756 | 0 | Uiso | 1 |
| C147 | C | 0.06445 | 0.66165 | 0.75541 | 0 | Uiso | 1 |
| C148 | C | 0.10435 | 0.61966 | 0.77344 | 0 | Uiso | 1 |
| C149 | C | 0.00992 | 0.38273 | 0.44694 | 0 | Uiso | 1 |
| C150 | C | 0.04182 | 0.33782 | 0.39599 | 0 | Uiso | 1 |
| C151 | C | 0.07983 | 0.32263 | 0.55891 | 0 | Uiso | 1 |
| C152 | C | 0.11121 | 0.27595 | 0.51759 | 0 | Uiso | 1 |
| C153 | C | 0.1546 | 0.26678 | 0.62684 | 0 | Uiso | 1 |
| C154 | C | 0.18354 | 0.21971 | 0.61411 | 0 | Uiso | 1 |
| C155 | C | 0.2276 | 0.21049 | 0.70892 | 0 | Uiso | 1 |
| C156 | C | 0.25578 | 0.16288 | 0.67713 | 0 | Uiso | 1 |
| F157 | F | 0.13453 | 0.61813 | 0.60831 | 0 | Uiso | 1 |
| F158 | F | 0.09533 | 0.58664 | 0.73378 | 0 | Uiso | 1 |
| F159 | F | 0.12261 | 0.61412 | 0.9933 | 0 | Uiso | 1 |
| F160 | F | 0.05153 | 0.67177 | 0.52166 | 0 | Uiso | 1 |
| F161 | F | 0.07436 | 0.69326 | 0.82694 | 0 | Uiso | 1 |
| F162 | F | 0.00895 | 0.69643 | 0.03775 | 0 | Uiso | 1 |
| F163 | F | 0.04637 | 0.62859 | 0.09016 | 0 | Uiso | 1 |
| F164 | F | 0.97337 | 0.68876 | 0.63536 | 0 | Uiso | 1 |
| F165 | F | 0.0162 | 0.62133 | 0.61204 | 0 | Uiso | 1 |
| F166 | F | 0.98856 | 0.60445 | 0.04787 | 0 | Uiso | 1 |
| F167 | F | 0.95012 | 0.67215 | 0.11386 | 0 | Uiso | 1 |
| F168 | F | 0.90173 | 0.63542 | 0.94242 | 0 | Uiso | 1 |
| F169 | F | 0.9054 | 0.68004 | 0.67994 | 0 | Uiso | 1 |
| F170 | F | 0.26274 | 0.15183 | 0.44202 | 0 | Uiso | 1 |
| F171 | F | 0.23939 | 0.13841 | 0.78975 | 0 | Uiso | 1 |
| F172 | F | 0.29623 | 0.15027 | 0.76806 | 0 | Uiso | 1 |
| F173 | F | 0.22577 | 0.2211 | 0.94625 | 0 | Uiso | 1 |
| F174 | F | 0.24589 | 0.23395 | 0.5906 | 0 | Uiso | 1 |
| F175 | F | 0.16629 | 0.19763 | 0.747 | 0 | Uiso | 1 |
| F176 | F | 0.18693 | 0.20627 | 0.3834 | 0 | Uiso | 1 |
| F177 | F | 0.14959 | 0.28005 | 0.85936 | 0 | Uiso | 1 |
| F178 | F | 0.17278 | 0.28977 | 0.50726 | 0 | Uiso | 1 |
| F179 | F | 0.11506 | 0.26897 | 0.27594 | 0 | Uiso | 1 |
| F180 | F | 0.0932 | 0.25103 | 0.61221 | 0 | Uiso | 1 |
| F181 | F | 0.0983 | 0.34918 | 0.51971 | 0 | Uiso | 1 |
| F182 | F | 0.06376 | 0.33007 | 0.78792 | 0 | Uiso | 1 |
| F183 | F | 0.13515 | 0.44725 | 0.84591 | 0 | Uiso | 1 |
| F184 | F | 0.14497 | 0.48958 | 0.5898 | 0 | Uiso | 1 |
| F185 | F | 0.12403 | 0.50951 | 0.96147 | 0 | Uiso | 1 |
| F186 | F | 0.2013 | 0.44729 | 0.14483 | 0 | Uiso | 1 |
| F187 | F | 0.22352 | 0.4213 | 0.78677 | 0 | Uiso | 1 |
| F188 | F | 0.18633 | 0.52831 | 0.97022 | 0 | Uiso | 1 |
| F189 | F | 0.20984 | 0.50233 | 0.61477 | 0 | Uiso | 1 |
| F190 | F | 0.28619 | 0.43946 | 0.82375 | 0 | Uiso | 1 |
| F191 | F | 0.26309 | 0.46574 | 0.17749 | 0 | Uiso | 1 |
| F192 | F | 0.24749 | 0.548 | 0.99269 | 0 | Uiso | 1 |
| F193 | F | 0.27212 | 0.52055 | 0.64282 | 0 | Uiso | 1 |
| F194 | F | 0.3483 | 0.45852 | 0.87806 | 0 | Uiso | 1 |
| C195 | C | 0.32008 | 0.49622 | 0.97134 | 0 | Uiso | 1 |
| F196 | F | 0.32232 | 0.49184 | 0.21379 | 0 | Uiso | 1 |
| F197 | F | 0.86781 | 0.45609 | 0.63577 | 0 | Uiso | 1 |
| F198 | F | 0.85774 | 0.39914 | 0.63906 | 0 | Uiso | 1 |
| F199 | F | 0.85827 | 0.43227 | 0.29998 | 0 | Uiso | 1 |
| F200 | F | 0.78263 | 0.50216 | 0.40818 | 0 | Uiso | 1 |
| F201 | F | 0.78405 | 0.48101 | 0.78352 | 0 | Uiso | 1 |
| F202 | F | 0.78119 | 0.40902 | 0.66598 | 0 | Uiso | 1 |
| F203 | F | 0.7909 | 0.41825 | 0.28165 | 0 | Uiso | 1 |
| F204 | F | 0.70912 | 0.49738 | 0.63359 | 0 | Uiso | 1 |
| F205 | F | 0.71753 | 0.48923 | 0.24477 | 0 | Uiso | 1 |
| F206 | F | 0.72514 | 0.40161 | 0.34996 | 0 | Uiso | 1 |
| F207 | F | 0.70165 | 0.42507 | 0.70953 | 0 | Uiso | 1 |
| F208 | F | 0.65681 | 0.46386 | 0.13661 | 0 | Uiso | 1 |
| C209 | C | 0.65752 | 0.46132 | 0.37977 | 0 | Uiso | 1 |
| F210 | F | 0.63621 | 0.50171 | 0.45657 | 0 | Uiso | 1 |
| H211 | H | 0.39187 | 0.22119 | 0.87455 | 0 | Uiso | 1 |
| H212 | H | 0.4307 | 0.14612 | 0.84326 | 0 | Uiso | 1 |
| H213 | H | 0.33284 | 0.13111 | 0.2417 | 0 | Uiso | 1 |
| H214 | H | 0.29412 | 0.20542 | 0.27596 | 0 | Uiso | 1 |
| H215 | H | 0.36946 | 0.06574 | 0.14632 | 0 | Uiso | 1 |
| H216 | H | 0.54001 | 0.95597 | 0.05692 | 0 | Uiso | 1 |
| H217 | H | 0.38731 | 0.99107 | 0.10717 | 0 | Uiso | 1 |
| H218 | H | 0.55752 | 0.8835 | 0.98987 | 0 | Uiso | 1 |
| H219 | H | 0.58447 | 0.82113 | 0.82682 | 0 | Uiso | 1 |
| H220 | H | 0.62707 | 0.74721 | 0.78492 | 0 | Uiso | 1 |
| H221 | H | 0.56287 | 0.72403 | 0.38624 | 0 | Uiso | 1 |
| H222 | H | 0.51988 | 0.7989 | 0.41922 | 0 | Uiso | 1 |
| H223 | H | 0.68133 | 0.68589 | 0.9636 | 0 | Uiso | 1 |
| H224 | H | 0.67581 | 0.57266 | 0.81332 | 0 | Uiso | 1 |
| H225 | H | 0.5671 | 0.67399 | 0.10998 | 0 | Uiso | 1 |
| H226 | H | 0.6447 | 0.53557 | 0.01208 | 0 | Uiso | 1 |
| H227 | H | 0.60569 | 0.49873 | 0.87257 | 0 | Uiso | 1 |
| H228 | H | 0.49627 | 0.61248 | 0.74733 | 0 | Uiso | 1 |
| H229 | H | 0.53475 | 0.65114 | 0.85466 | 0 | Uiso | 1 |
| H230 | H | 0.48591 | 0.57223 | 0.48065 | 0 | Uiso | 1 |
| H231 | H | 0.52226 | 0.40792 | 0.35599 | 0 | Uiso | 1 |
| H232 | H | 0.42537 | 0.55772 | 0.35578 | 0 | Uiso | 1 |
| H233 | H | 0.46529 | 0.3942 | 0.16669 | 0 | Uiso | 1 |
| H234 | H | 0.71386 | 0.66539 | 0.56995 | 0 | Uiso | 1 |
| H235 | H | 0.78545 | 0.63779 | 0.39178 | 0 | Uiso | 1 |
| H236 | H | 0.82159 | 0.52513 | 0.79241 | 0 | Uiso | 1 |
| H237 | H | 0.75041 | 0.55122 | 0.97118 | 0 | Uiso | 1 |
| H238 | H | 0.85171 | 0.51029 | 0.34626 | 0 | Uiso | 1 |
| H239 | H | 0.99724 | 0.53497 | 0.26564 | 0 | Uiso | 1 |
| H240 | H | 0.91933 | 0.44912 | 0.28859 | 0 | Uiso | 1 |
| H241 | H | 0.06514 | 0.47374 | 0.25159 | 0 | Uiso | 1 |
| H242 | H | 0.12564 | 0.42775 | 0.455 | 0 | Uiso | 1 |
| H243 | H | 0.20271 | 0.38877 | 0.45372 | 0 | Uiso | 1 |
| H244 | H | 0.19514 | 0.31586 | 0.88184 | 0 | Uiso | 1 |
| H245 | H | 0.11712 | 0.35385 | 0.90285 | 0 | Uiso | 1 |
| H246 | H | 0.25913 | 0.38342 | 0.18951 | 0 | Uiso | 1 |
| H247 | H | 0.37435 | 0.27226 | 0.15919 | 0 | Uiso | 1 |
| H248 | H | 0.25457 | 0.26804 | 0.09861 | 0 | Uiso | 1 |
| H249 | H | 0.30059 | 0.39309 | 0.48944 | 0 | Uiso | 1 |
| H250 | H | 0.33707 | 0.43697 | 0.52216 | 0 | Uiso | 1 |
| H251 | H | 0.42513 | 0.3695 | 0.98211 | 0 | Uiso | 1 |
| H252 | H | 0.38791 | 0.32666 | 0.93575 | 0 | Uiso | 1 |
| H253 | H | 0.46983 | 0.88191 | 0.79366 | 0 | Uiso | 1 |
| H254 | H | 0.41502 | 0.91464 | 0.74286 | 0 | Uiso | 1 |
| H255 | H | 0.393 | 0.86565 | 0.90645 | 0 | Uiso | 1 |
| H256 | H | 0.439 | 0.84113 | 0.08859 | 0 | Uiso | 1 |
| H257 | H | 0.51957 | 0.02647 | 0.40838 | 0 | Uiso | 1 |
| H258 | H | 0.464 | 0.06028 | 0.3917 | 0 | Uiso | 1 |
| H259 | H | 0.49516 | 0.09906 | 0.06963 | 0 | Uiso | 1 |
| H260 | H | 0.53959 | 0.07671 | 0.26108 | 0 | Uiso | 1 |
| H261 | H | 0.40241 | 0.47964 | 0.89006 | 0 | Uiso | 1 |
| H262 | H | 0.39638 | 0.53163 | 0.85529 | 0 | Uiso | 1 |
| H263 | H | 0.33247 | 0.53382 | 0.70922 | 0 | Uiso | 1 |
| H264 | H | 0.31846 | 0.55624 | 0.99763 | 0 | Uiso | 1 |
| H265 | H | 0.57549 | 0.49386 | 0.3288 | 0 | Uiso | 1 |
| H266 | H | 0.587 | 0.44784 | 0.16807 | 0 | Uiso | 1 |
| H267 | H | 0.64695 | 0.40666 | 0.3968 | 0 | Uiso | 1 |
| H268 | H | 0.62803 | 0.43909 | 0.647 | 0 | Uiso | 1 |
| H269 | H | 0.9089 | 0.57399 | 0.68813 | 0 | Uiso | 1 |
| H270 | H | 0.87805 | 0.62295 | 0.54515 | 0 | Uiso | 1 |
| H271 | H | 0.95199 | 0.6221 | 0.43996 | 0 | Uiso | 1 |
| H272 | H | 0.97271 | 0.5815 | 0.65976 | 0 | Uiso | 1 |
| H273 | H | 0.98069 | 0.38332 | 0.53058 | 0 | Uiso | 1 |
| H274 | H | 0.02332 | 0.39675 | 0.57486 | 0 | Uiso | 1 |
| H275 | H | 0.05396 | 0.33607 | 0.21277 | 0 | Uiso | 1 |
| H276 | H | 0.02644 | 0.31693 | 0.41486 | 0 | Uiso | 1 |

TABLE S2
Fractional atomic coordinates for the unit cell of CityU-14.
CityU-14: space group P1
a = 36.1192 Å, b = 36.3344 Å, c = 5.5680 Å
α = 88.1417°, β = 92.1170°, γ = 61.2965°

| | | | | | U_iso | | |
|---|---|---|---|---|---|---|---|
| | | | | | or | | occu- |
| label | atom | fract_x | fract_y | fract_z | equiv | adp_type | pancy |
| C1 | C | 0.34467 | 0.22895 | 0.87604 | 0 | Uiso | 1 |
| C2 | C | 0.3889 | 0.20986 | 0.90927 | 0 | Uiso | 1 |
| C3 | C | 0.41314 | 0.16608 | 0.92732 | 0 | Uiso | 1 |
| C4 | C | 0.39405 | 0.14056 | 0.91051 | 0 | Uiso | 1 |
| C5 | C | 0.34967 | 0.15963 | 0.8849 | 0 | Uiso | 1 |
| C6 | C | 0.32531 | 0.20339 | 0.86779 | 0 | Uiso | 1 |
| N7 | N | 0.42106 | 0.09595 | 0.9049 | 0 | Uiso | 1 |
| C8 | C | 0.40998 | 0.06721 | 0.8744 | 0 | Uiso | 1 |
| C9 | C | 0.44217 | 0.02257 | 0.85697 | 0 | Uiso | 1 |
| C10 | C | 0.4853 | 0.01017 | 0.82899 | 0 | Uiso | 1 |
| C11 | C | 0.51468 | 0.96749 | 0.82767 | 0 | Uiso | 1 |
| C12 | C | 0.50172 | 0.93686 | 0.84079 | 0 | Uiso | 1 |
| C13 | C | 0.45824 | 0.94896 | 0.84513 | 0 | Uiso | 1 |
| C14 | C | 0.42886 | 0.99186 | 0.85899 | 0 | Uiso | 1 |
| C15 | C | 0.5343 | 0.89231 | 0.84108 | 0 | Uiso | 1 |
| N16 | N | 0.5277 | 0.8633 | 0.9394 | 0 | Uiso | 1 |
| C17 | C | 0.55515 | 0.8189 | 0.94164 | 0 | Uiso | 1 |
| C18 | C | 0.59854 | 0.80096 | 0.90275 | 0 | Uiso | 1 |
| C19 | C | 0.62277 | 0.75749 | 0.88428 | 0 | Uiso | 1 |
| C20 | C | 0.60386 | 0.73155 | 0.90075 | 0 | Uiso | 1 |
| C21 | C | 0.56086 | 0.74967 | 0.94943 | 0 | Uiso | 1 |
| C22 | C | 0.53716 | 0.79285 | 0.97414 | 0 | Uiso | 1 |
| C23 | C | 0.62784 | 0.68637 | 0.85448 | 0 | Uiso | 1 |
| N24 | N | 0.67032 | 0.66707 | 0.83459 | 0 | Uiso | 1 |
| C25 | C | 0.69148 | 0.62619 | 0.77866 | 0 | Uiso | 1 |
| N26 | N | 0.66975 | 0.60501 | 0.74055 | 0 | Uiso | 1 |
| C27 | C | 0.62752 | 0.62379 | 0.76347 | 0 | Uiso | 1 |
| N28 | N | 0.60683 | 0.66439 | 0.82087 | 0 | Uiso | 1 |
| C29 | C | 0.60394 | 0.60062 | 0.71973 | 0 | Uiso | 1 |
| C30 | C | 0.62243 | 0.56321 | 0.59949 | 0 | Uiso | 1 |
| C31 | C | 0.59971 | 0.54208 | 0.55117 | 0 | Uiso | 1 |
| C32 | C | 0.55827 | 0.55765 | 0.61917 | 0 | Uiso | 1 |
| C33 | C | 0.53975 | 0.59465 | 0.7445 | 0 | Uiso | 1 |
| C34 | C | 0.56256 | 0.61594 | 0.79454 | 0 | Uiso | 1 |
| N35 | N | 0.53728 | 0.53344 | 0.5715 | 0 | Uiso | 1 |
| O36 | O | 0.44612 | 0.9175 | 0.83106 | 0 | Uiso | 1 |
| O37 | O | 0.49943 | 0.03992 | 0.81523 | 0 | Uiso | 1 |
| C38 | C | 0.4967 | 0.54775 | 0.54188 | 0 | Uiso | 1 |
| C39 | C | 0.47904 | 0.51869 | 0.50877 | 0 | Uiso | 1 |
| C40 | C | 0.50491 | 0.47455 | 0.50137 | 0 | Uiso | 1 |
| C41 | C | 0.48658 | 0.4485 | 0.49884 | 0 | Uiso | 1 |
| C42 | C | 0.44257 | 0.46515 | 0.4964 | 0 | Uiso | 1 |
| C43 | C | 0.41635 | 0.50946 | 0.4798 | 0 | Uiso | 1 |
| C44 | C | 0.43481 | 0.53549 | 0.49208 | 0 | Uiso | 1 |
| C45 | C | 0.42614 | 0.43515 | 0.52513 | 0 | Uiso | 1 |
| N46 | N | 0.3899 | 0.44635 | 0.61554 | 0 | Uiso | 1 |
| O47 | O | 0.54903 | 0.45536 | 0.51617 | 0 | Uiso | 1 |
| O48 | O | 0.37205 | 0.52874 | 0.45655 | 0 | Uiso | 1 |
| C49 | C | 0.7381 | 0.60409 | 0.76859 | 0 | Uiso | 1 |
| C50 | C | 0.76246 | 0.62047 | 0.87597 | 0 | Uiso | 1 |
| C51 | C | 0.8066 | 0.59732 | 0.88724 | 0 | Uiso | 1 |
| C52 | C | 0.82702 | 0.5575 | 0.79499 | 0 | Uiso | 1 |
| C53 | C | 0.80304 | 0.54186 | 0.67701 | 0 | Uiso | 1 |
| C54 | C | 0.75886 | 0.56506 | 0.66359 | 0 | Uiso | 1 |
| N55 | N | 0.87218 | 0.53442 | 0.82261 | 0 | Uiso | 1 |
| C56 | C | 0.89551 | 0.4939 | 0.83621 | 0 | Uiso | 1 |
| C57 | C | 0.94205 | 0.47449 | 0.87983 | 0 | Uiso | 1 |
| C58 | C | 0.96284 | 0.499 | 0.89619 | 0 | Uiso | 1 |
| C59 | C | 0.00611 | 0.4795 | 0.95832 | 0 | Uiso | 1 |
| C60 | C | 0.02944 | 0.436 | 0.00756 | 0 | Uiso | 1 |
| C61 | C | 0.00946 | 0.41107 | 0.97826 | 0 | Uiso | 1 |
| C62 | C | 0.96607 | 0.43048 | 0.91557 | 0 | Uiso | 1 |
| C63 | C | 0.07317 | 0.41902 | 0.10747 | 0 | Uiso | 1 |
| N64 | N | 0.08689 | 0.39277 | 0.29447 | 0 | Uiso | 1 |
| C65 | C | 0.12692 | 0.37976 | 0.42101 | 0 | Uiso | 1 |
| C66 | C | 0.14255 | 0.40792 | 0.4592 | 0 | Uiso | 1 |
| C67 | C | 0.18282 | 0.39355 | 0.57096 | 0 | Uiso | 1 |
| C68 | C | 0.20775 | 0.35095 | 0.64742 | 0 | Uiso | 1 |
| C69 | C | 0.19045 | 0.32381 | 0.62577 | 0 | Uiso | 1 |
| C70 | C | 0.15033 | 0.33839 | 0.51438 | 0 | Uiso | 1 |
| C71 | C | 0.25262 | 0.33393 | 0.7292 | 0 | Uiso | 1 |
| N72 | N | 0.27127 | 0.35847 | 0.71963 | 0 | Uiso | 1 |
| C73 | C | 0.31354 | 0.34168 | 0.76828 | 0 | Uiso | 1 |
| N74 | N | 0.33722 | 0.29986 | 0.82428 | 0 | Uiso | 1 |
| C75 | C | 0.31881 | 0.27505 | 0.83473 | 0 | Uiso | 1 |
| N76 | N | 0.27663 | 0.29232 | 0.78585 | 0 | Uiso | 1 |
| C77 | C | 0.3339 | 0.36834 | 0.74155 | 0 | Uiso | 1 |
| C78 | C | 0.30912 | 0.41204 | 0.69893 | 0 | Uiso | 1 |
| C79 | C | 0.32846 | 0.43692 | 0.66488 | 0 | Uiso | 1 |
| C80 | C | 0.37221 | 0.41909 | 0.66308 | 0 | Uiso | 1 |
| C81 | C | 0.39724 | 0.37555 | 0.71109 | 0 | Uiso | 1 |
| C82 | C | 0.37822 | 0.35034 | 0.74986 | 0 | Uiso | 1 |
| C83 | C | 0.40355 | 0.9283 | 0.75929 | 0 | Uiso | 1 |
| C84 | C | 0.40239 | 0.88773 | 0.70091 | 0 | Uiso | 1 |
| C85 | C | 0.42658 | 0.86747 | 0.47811 | 0 | Uiso | 1 |
| C86 | C | 0.41866 | 0.83129 | 0.37975 | 0 | Uiso | 1 |
| C87 | C | 0.43073 | 0.79591 | 0.57817 | 0 | Uiso | 1 |
| C88 | C | 0.43372 | 0.75539 | 0.46784 | 0 | Uiso | 1 |
| C89 | C | 0.48045 | 0.72303 | 0.44249 | 0 | Uiso | 1 |
| C90 | C | 0.48101 | 0.6847 | 0.31503 | 0 | Uiso | 1 |
| C91 | C | 0.51887 | 0.04121 | 0.59762 | 0 | Uiso | 1 |
| C92 | C | 0.53092 | 0.0763 | 0.60922 | 0 | Uiso | 1 |
| C93 | C | 0.4994 | 0.11663 | 0.46394 | 0 | Uiso | 1 |
| C94 | C | 0.50965 | 0.15303 | 0.51593 | 0 | Uiso | 1 |
| C95 | C | 0.47556 | 0.19479 | 0.38638 | 0 | Uiso | 1 |
| C96 | C | 0.48259 | 0.23129 | 0.4775 | 0 | Uiso | 1 |
| C97 | C | 0.46298 | 0.26953 | 0.29078 | 0 | Uiso | 1 |
| C98 | C | 0.49965 | 0.26739 | 0.13903 | 0 | Uiso | 1 |
| C99 | C | 0.35546 | 0.52465 | 0.22737 | 0 | Uiso | 1 |
| C100 | C | 0.30706 | 0.54382 | 0.23126 | 0 | Uiso | 1 |
| C101 | C | 0.24107 | 0.54522 | 0.00125 | 0 | Uiso | 1 |
| C102 | C | 0.22241 | 0.53692 | 0.23055 | 0 | Uiso | 1 |
| C103 | C | 0.18235 | 0.53252 | 0.16846 | 0 | Uiso | 1 |
| C104 | C | 0.19588 | 0.48575 | 0.14147 | 0 | Uiso | 1 |
| C105 | C | 0.15694 | 0.4831 | 0.02532 | 0 | Uiso | 1 |
| C106 | C | 0.56731 | 0.44999 | 0.28777 | 0 | Uiso | 1 |
| C107 | C | 0.61513 | 0.43264 | 0.33334 | 0 | Uiso | 1 |
| C108 | C | 0.68212 | 0.43074 | 0.18474 | 0 | Uiso | 1 |
| C109 | C | 0.70143 | 0.43876 | 0.95816 | 0 | Uiso | 1 |
| C110 | C | 0.74244 | 0.44106 | 0.03204 | 0 | Uiso | 1 |
| C111 | C | 0.73156 | 0.48748 | 0.0441 | 0 | Uiso | 1 |
| C112 | C | 0.77127 | 0.48843 | 0.16238 | 0 | Uiso | 1 |
| F113 | F | 0.52516 | 0.22773 | 0.05914 | 0 | Uiso | 1 |
| F114 | F | 0.52539 | 0.27897 | 0.273 | 0 | Uiso | 1 |
| F115 | F | 0.48365 | 0.29597 | 0.93962 | 0 | Uiso | 1 |
| F116 | F | 0.44436 | 0.30788 | 0.40673 | 0 | Uiso | 1 |
| F117 | F | 0.42877 | 0.27148 | 0.14914 | 0 | Uiso | 1 |
| F118 | F | 0.52525 | 0.21858 | 0.53222 | 0 | Uiso | 1 |
| F119 | F | 0.463 | 0.24355 | 0.69578 | 0 | Uiso | 1 |
| F120 | F | 0.47803 | 0.19228 | 0.13856 | 0 | Uiso | 1 |
| F121 | F | 0.43411 | 0.20274 | 0.42789 | 0 | Uiso | 1 |
| F122 | F | 0.55026 | 0.14167 | 0.44118 | 0 | Uiso | 1 |
| F123 | F | 0.51172 | 0.15643 | 0.76436 | 0 | Uiso | 1 |
| F124 | F | 0.45903 | 0.12539 | 0.52294 | 0 | Uiso | 1 |
| F125 | F | 0.5025 | 0.10705 | 0.2228 | 0 | Uiso | 1 |
| F126 | F | 0.52239 | 0.65265 | 0.2921 | 0 | Uiso | 1 |
| F127 | F | 0.46168 | 0.66557 | 0.44239 | 0 | Uiso | 1 |
| F128 | F | 0.46237 | 0.69518 | 0.08359 | 0 | Uiso | 1 |
| F129 | F | 0.50147 | 0.74069 | 0.31224 | 0 | Uiso | 1 |
| F130 | F | 0.50295 | 0.70978 | 0.66951 | 0 | Uiso | 1 |
| F131 | F | 0.41561 | 0.73764 | 0.61785 | 0 | Uiso | 1 |
| F132 | F | 0.41049 | 0.76462 | 0.24748 | 0 | Uiso | 1 |
| F133 | F | 0.46805 | 0.78792 | 0.70753 | 0 | Uiso | 1 |
| F134 | F | 0.39969 | 0.81115 | 0.74626 | 0 | Uiso | 1 |
| F135 | F | 0.37604 | 0.8484 | 0.29975 | 0 | Uiso | 1 |
| F136 | F | 0.44221 | 0.81532 | 0.17836 | 0 | Uiso | 1 |
| F137 | F | 0.41374 | 0.89994 | 0.30004 | 0 | Uiso | 1 |
| F138 | F | 0.46942 | 0.85317 | 0.53082 | 0 | Uiso | 1 |
| O139 | O | 0.94008 | 0.54285 | 0.89258 | 0 | Uiso | 1 |
| O140 | O | 0.03169 | 0.3672 | 0.01562 | 0 | Uiso | 1 |
| C141 | C | 0.94486 | 0.56453 | 0.68779 | 0 | Uiso | 1 |
| C142 | C | 0.9242 | 0.61154 | 0.73417 | 0 | Uiso | 1 |
| C143 | C | 0.93954 | 0.62298 | 0.96736 | 0 | Uiso | 1 |
| C144 | C | 0.98707 | 0.61255 | 0.97147 | 0 | Uiso | 1 |
| C145 | C | 0.9995 | 0.62204 | 0.22122 | 0 | Uiso | 1 |
| C146 | C | 0.04248 | 0.62251 | 0.21266 | 0 | Uiso | 1 |
| C147 | C | 0.03484 | 0.66782 | 0.18339 | 0 | Uiso | 1 |
| C148 | C | 0.07688 | 0.66575 | 0.10926 | 0 | Uiso | 1 |
| C149 | C | 0.05247 | 0.346 | 0.80883 | 0 | Uiso | 1 |
| C150 | C | 0.0771 | 0.29819 | 0.86736 | 0 | Uiso | 1 |
| C151 | C | 0.11501 | 0.28464 | 0.04903 | 0 | Uiso | 1 |
| C152 | C | 0.14604 | 0.23585 | 0.05489 | 0 | Uiso | 1 |
| C153 | C | 0.1884 | 0.22646 | 0.19006 | 0 | Uiso | 1 |
| C154 | C | 0.21643 | 0.17803 | 0.23045 | 0 | Uiso | 1 |
| C155 | C | 0.25889 | 0.16822 | 0.35988 | 0 | Uiso | 1 |
| C156 | C | 0.2878 | 0.1192 | 0.36544 | 0 | Uiso | 1 |
| F157 | F | 0.07481 | 0.7048 | 0.12533 | 0 | Uiso | 1 |
| F158 | F | 0.08484 | 0.65491 | 0.87257 | 0 | Uiso | 1 |
| F159 | F | 0.11169 | 0.63727 | 0.25512 | 0 | Uiso | 1 |
| F160 | F | 0.02234 | 0.68783 | 0.4002 | 0 | Uiso | 1 |
| F161 | F | 0.00191 | 0.69169 | 0.01049 | 0 | Uiso | 1 |
| F162 | F | 0.06692 | 0.59835 | 0.03131 | 0 | Uiso | 1 |
| F163 | F | 0.06672 | 0.60408 | 0.42671 | 0 | Uiso | 1 |
| F164 | F | 0.96777 | 0.65942 | 0.30418 | 0 | Uiso | 1 |
| F165 | F | 0.00367 | 0.59025 | 0.38736 | 0 | Uiso | 1 |
| F166 | F | 0.01343 | 0.57005 | 0.92968 | 0 | Uiso | 1 |
| F167 | F | 0.99305 | 0.63569 | 0.78617 | 0 | Uiso | 1 |
| F168 | F | 0.93137 | 0.60408 | 0.16511 | 0 | Uiso | 1 |
| F169 | F | 0.91305 | 0.66576 | 0.98094 | 0 | Uiso | 1 |
| F170 | F | 0.32519 | 0.10629 | 0.50732 | 0 | Uiso | 1 |
| F171 | F | 0.30078 | 0.10519 | 0.13795 | 0 | Uiso | 1 |
| F172 | F | 0.26882 | 0.09691 | 0.45781 | 0 | Uiso | 1 |
| F173 | F | 0.27873 | 0.18835 | 0.24147 | 0 | Uiso | 1 |
| F174 | F | 0.25344 | 0.18301 | 0.59381 | 0 | Uiso | 1 |
| F175 | F | 0.22343 | 0.1587 | 0.01157 | 0 | Uiso | 1 |
| F176 | F | 0.19638 | 0.16097 | 0.36922 | 0 | Uiso | 1 |
| F177 | F | 0.20909 | 0.24352 | 0.05397 | 0 | Uiso | 1 |
| F178 | F | 0.18144 | 0.24592 | 0.40977 | 0 | Uiso | 1 |
| F179 | F | 0.12648 | 0.21483 | 0.16265 | 0 | Uiso | 1 |
| F180 | F | 0.15281 | 0.22249 | 0.81975 | 0 | Uiso | 1 |
| F181 | F | 0.13492 | 0.30805 | 0.98874 | 0 | Uiso | 1 |
| F182 | F | 0.09863 | 0.29677 | 0.27339 | 0 | Uiso | 1 |
| F183 | F | 0.16502 | 0.44143 | 0.02025 | 0 | Uiso | 1 |
| F184 | F | 0.14757 | 0.49885 | 0.78841 | 0 | Uiso | 1 |
| F185 | F | 0.12084 | 0.50354 | 0.15251 | 0 | Uiso | 1 |
| F186 | F | 0.23157 | 0.4666 | 0.00272 | 0 | Uiso | 1 |
| F187 | F | 0.20975 | 0.46327 | 0.36772 | 0 | Uiso | 1 |
| F188 | F | 0.15982 | 0.55765 | 0.96381 | 0 | Uiso | 1 |
| F189 | F | 0.15511 | 0.54707 | 0.35477 | 0 | Uiso | 1 |
| F190 | F | 0.25283 | 0.50206 | 0.37008 | 0 | Uiso | 1 |
| F191 | F | 0.21242 | 0.57074 | 0.37469 | 0 | Uiso | 1 |
| F192 | F | 0.22359 | 0.58845 | 0.94644 | 0 | Uiso | 1 |
| F193 | F | 0.23196 | 0.52841 | 0.80059 | 0 | Uiso | 1 |
| F194 | F | 0.3072 | 0.53014 | 0.82571 | 0 | Uiso | 1 |
| C195 | C | 0.29058 | 0.52543 | 0.03787 | 0 | Uiso | 1 |
| F196 | F | 0.30999 | 0.48239 | 0.09039 | 0 | Uiso | 1 |
| F197 | F | 0.76502 | 0.52942 | 0.16379 | 0 | Uiso | 1 |
| F198 | F | 0.80796 | 0.4655 | 0.0436 | 0 | Uiso | 1 |
| F199 | F | 0.77934 | 0.4739 | 0.40195 | 0 | Uiso | 1 |
| F200 | F | 0.69552 | 0.50997 | 0.17584 | 0 | Uiso | 1 |
| F201 | F | 0.72028 | 0.5078 | 0.8117 | 0 | Uiso | 1 |
| F202 | F | 0.77262 | 0.42216 | 0.86235 | 0 | Uiso | 1 |
| F203 | F | 0.76135 | 0.41821 | 0.24923 | 0 | Uiso | 1 |
| F204 | F | 0.7109 | 0.40581 | 0.80687 | 0 | Uiso | 1 |
| F205 | F | 0.67192 | 0.4751 | 0.82437 | 0 | Uiso | 1 |
| F206 | F | 0.684 | 0.45385 | 0.37545 | 0 | Uiso | 1 |
| F207 | F | 0.70393 | 0.38848 | 0.26823 | 0 | Uiso | 1 |
| F208 | F | 0.62924 | 0.42472 | 0.92286 | 0 | Uiso | 1 |
| C209 | C | 0.6347 | 0.4438 | 0.12296 | 0 | Uiso | 1 |
| F210 | F | 0.61071 | 0.48697 | 0.07338 | 0 | Uiso | 1 |
| H211 | H | 0.40485 | 0.22874 | 0.91396 | 0 | Uiso | 1 |
| H212 | H | 0.44723 | 0.15158 | 0.94307 | 0 | Uiso | 1 |
| H213 | H | 0.33325 | 0.14149 | 0.87408 | 0 | Uiso | 1 |
| H214 | H | 0.29104 | 0.21732 | 0.84466 | 0 | Uiso | 1 |
| H215 | H | 0.37705 | 0.07512 | 0.85906 | 0 | Uiso | 1 |
| H216 | H | 0.54793 | 0.95822 | 0.82298 | 0 | Uiso | 1 |
| H217 | H | 0.39573 | 0.0015 | 0.8741 | 0 | Uiso | 1 |
| H218 | H | 0.56336 | 0.88446 | 0.75495 | 0 | Uiso | 1 |
| H219 | H | 0.61372 | 0.82022 | 0.88551 | 0 | Uiso | 1 |
| H220 | H | 0.65598 | 0.74423 | 0.84911 | 0 | Uiso | 1 |
| H221 | H | 0.54507 | 0.73071 | 0.96137 | 0 | Uiso | 1 |
| H222 | H | 0.50393 | 0.80686 | 0.00691 | 0 | Uiso | 1 |
| H223 | H | 0.65456 | 0.55014 | 0.54293 | 0 | Uiso | 1 |
| H224 | H | 0.61457 | 0.51322 | 0.46151 | 0 | Uiso | 1 |
| H225 | H | 0.50802 | 0.60708 | 0.80488 | 0 | Uiso | 1 |
| H226 | H | 0.54791 | 0.6445 | 0.89104 | 0 | Uiso | 1 |
| H227 | H | 0.47509 | 0.5813 | 0.54385 | 0 | Uiso | 1 |
| H228 | H | 0.50742 | 0.41482 | 0.50942 | 0 | Uiso | 1 |
| H229 | H | 0.41398 | 0.56911 | 0.49054 | 0 | Uiso | 1 |
| H230 | H | 0.44583 | 0.403 | 0.47704 | 0 | Uiso | 1 |
| H231 | H | 0.74741 | 0.65044 | 0.95832 | 0 | Uiso | 1 |
| H232 | H | 0.82532 | 0.60949 | 0.97597 | 0 | Uiso | 1 |
| H233 | H | 0.81818 | 0.51172 | 0.59726 | 0 | Uiso | 1 |
| H234 | H | 0.74088 | 0.552 | 0.57569 | 0 | Uiso | 1 |
| H235 | H | 0.88106 | 0.47374 | 0.83212 | 0 | Uiso | 1 |
| H236 | H | 0.02054 | 0.4993 | 0.98706 | 0 | Uiso | 1 |
| H237 | H | 0.95112 | 0.4109 | 0.90283 | 0 | Uiso | 1 |
| H238 | H | 0.09204 | 0.43257 | 0.03901 | 0 | Uiso | 1 |
| H239 | H | 0.12383 | 0.44084 | 0.40095 | 0 | Uiso | 1 |
| H240 | H | 0.1948 | 0.41551 | 0.59521 | 0 | Uiso | 1 |
| H241 | H | 0.20827 | 0.29088 | 0.68888 | 0 | Uiso | 1 |
| H242 | H | 0.13786 | 0.31714 | 0.49431 | 0 | Uiso | 1 |
| H243 | H | 0.27477 | 0.42696 | 0.69008 | 0 | Uiso | 1 |
| H244 | H | 0.30966 | 0.47048 | 0.63994 | 0 | Uiso | 1 |
| H245 | H | 0.43135 | 0.36076 | 0.71705 | 0 | Uiso | 1 |
| H246 | H | 0.39817 | 0.31651 | 0.77888 | 0 | Uiso | 1 |
| H247 | H | 0.39162 | 0.9509 | 0.59844 | 0 | Uiso | 1 |
| H248 | H | 0.38272 | 0.94296 | 0.91008 | 0 | Uiso | 1 |
| H249 | H | 0.36914 | 0.89447 | 0.67242 | 0 | Uiso | 1 |
| H250 | H | 0.41756 | 0.86518 | 0.85689 | 0 | Uiso | 1 |
| H251 | H | 0.4969 | 0.04672 | 0.43669 | 0 | Uiso | 1 |
| H252 | H | 0.54813 | 0.01092 | 0.57967 | 0 | Uiso | 1 |
| H253 | H | 0.56257 | 0.06563 | 0.53873 | 0 | Uiso | 1 |
| H254 | H | 0.5325 | 0.08343 | 0.80039 | 0 | Uiso | 1 |
| H255 | H | 0.36992 | 0.49084 | 0.18332 | 0 | Uiso | 1 |
| H256 | H | 0.36323 | 0.54116 | 0.08125 | 0 | Uiso | 1 |
| H257 | H | 0.29188 | 0.57847 | 0.20138 | 0 | Uiso | 1 |
| H258 | H | 0.29938 | 0.53622 | 0.40941 | 0 | Uiso | 1 |
| H259 | H | 0.55268 | 0.48075 | 0.17968 | 0 | Uiso | 1 |
| H260 | H | 0.56146 | 0.42756 | 0.1818 | 0 | Uiso | 1 |
| H261 | H | 0.63094 | 0.39802 | 0.36643 | 0 | Uiso | 1 |
| H262 | H | 0.62094 | 0.44576 | 0.49846 | 0 | Uiso | 1 |
| H263 | H | 0.92836 | 0.56028 | 0.52588 | 0 | Uiso | 1 |
| H264 | H | 0.97853 | 0.55199 | 0.64425 | 0 | Uiso | 1 |
| H265 | H | 0.88989 | 0.62322 | 0.74688 | 0 | Uiso | 1 |
| H266 | H | 0.92794 | 0.62944 | 0.58079 | 0 | Uiso | 1 |
| H267 | H | 0.07424 | 0.35756 | 0.74682 | 0 | Uiso | 1 |
| H268 | H | 0.02873 | 0.35268 | 0.65677 | 0 | Uiso | 1 |
| H269 | H | 0.08839 | 0.28258 | 0.69709 | 0 | Uiso | 1 |
| H270 | H | 0.05564 | 0.28767 | 0.9439 | 0 | Uiso | 1 |

The ordered structure of both COFs was further confirmed by ultralow-dose high resolution transmission electron microscope (HRTEM) images (FIG. 38). The regular lattice fringes with spacing of 3.01 nm for CityU-13 and 3.04 nm for CityU-14 in the HRTEM images were ascribed to the (100) plane (FIG. 25c). Moreover, the lattice spacing of both COFs were consistent with the pore diameters of CityU-13 (27.98 Å) and CityU-14 (28.33 Å) in the simulated COF structures (FIG. 25b), indicating that the grid stacking along the c axis led to the distinct one-dimensional open channels with a uniform diameter.^{[15]}

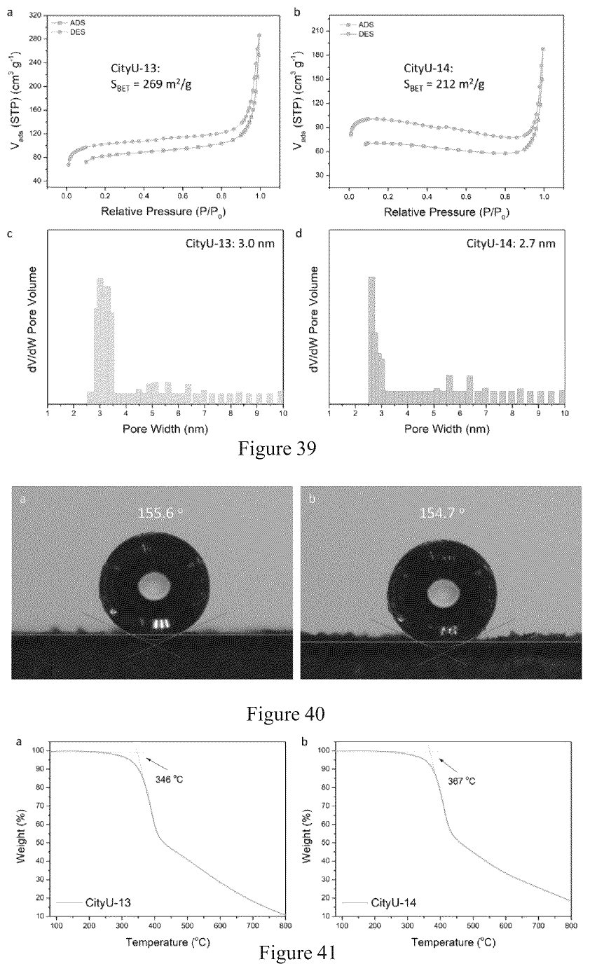

CityU-13 and CityU-14 showed the Brunauer-Emmett-Teller (BET) surface areas of 269 and 212 m²/g, respectively (FIG. 39). The pore sizes of CityU-13 and CityU-14 were around 3.0 and 2.7 nm, respectively, as derived from the corresponding N₂ adsorption isotherm using the nonlocal density functional theory method. The pore sizes of both COFs agreed well with theoretical structural analysis (FIG. 25b), further confirming their layered stacking model.

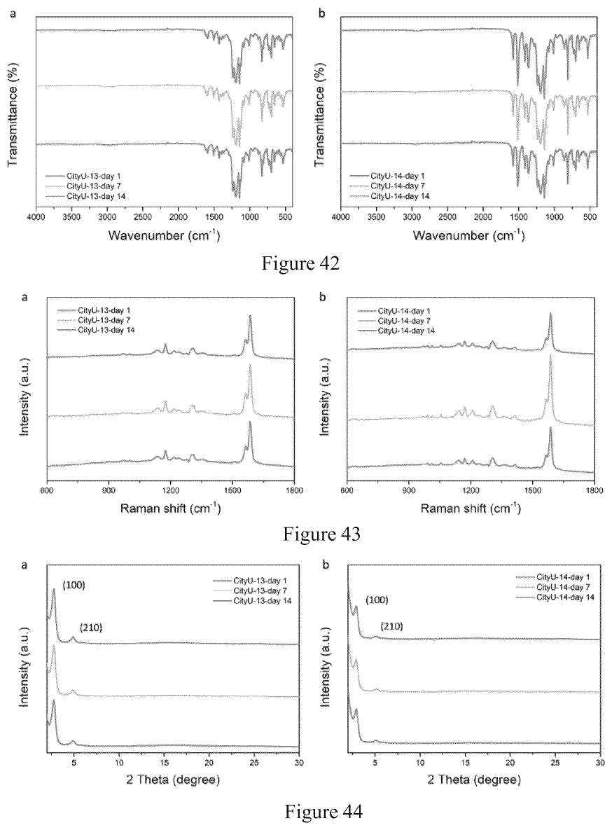

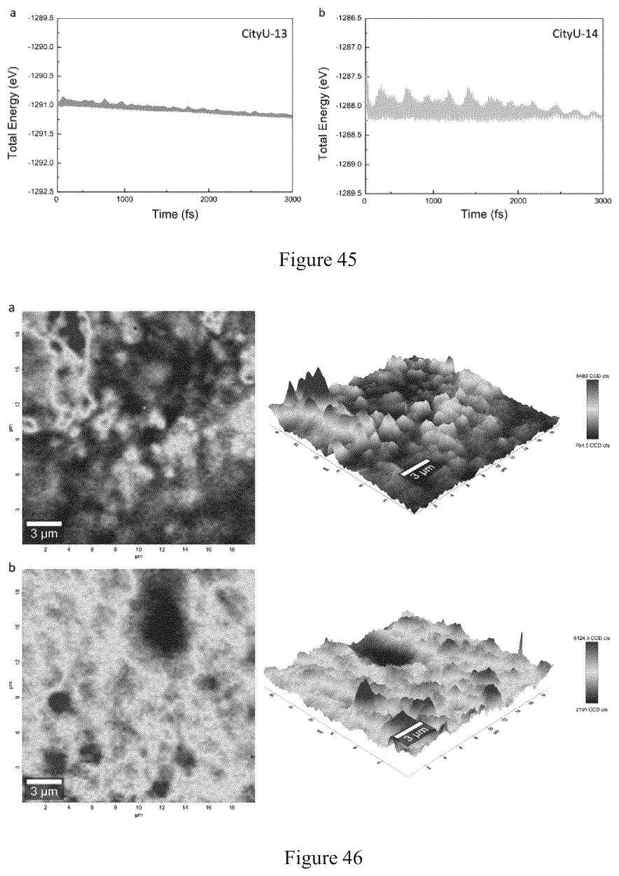

Since fluorinated chains are uniformly distributed in order pores of both COFs, the superhydrophobicity of both COFs were further studied. High water contact angles of 155.6° and 154.7° were obtained for CityU-13 and CityU-14, respectively (FIG. 40), which were even higher than poly(tetrafluoroethylene) (PTFE, 120°).^{[16]} Besides, both COFs displayed good thermal stability since there was no obvious weight loss until 346° C. for CityU-13 (FIG. 41a) and 367° C. for CityU-14 (FIG. 41b), demonstrating their great potentials in practical applications. According to the FTIR, Raman spectra and PXRD patterns over extended periods (FIGS. 42-44), both COFs demonstrated remarkable consistency in their physical properties, indicating their remarkable stability. Furthermore, AIMD simulations at a temperature of 300K were conducted to assess the thermal resilience. These simulations revealed that even in a single-layer configuration, both COFs maintained their thermal stability (FIG. 45), further validating their potential for use in demanding environments.

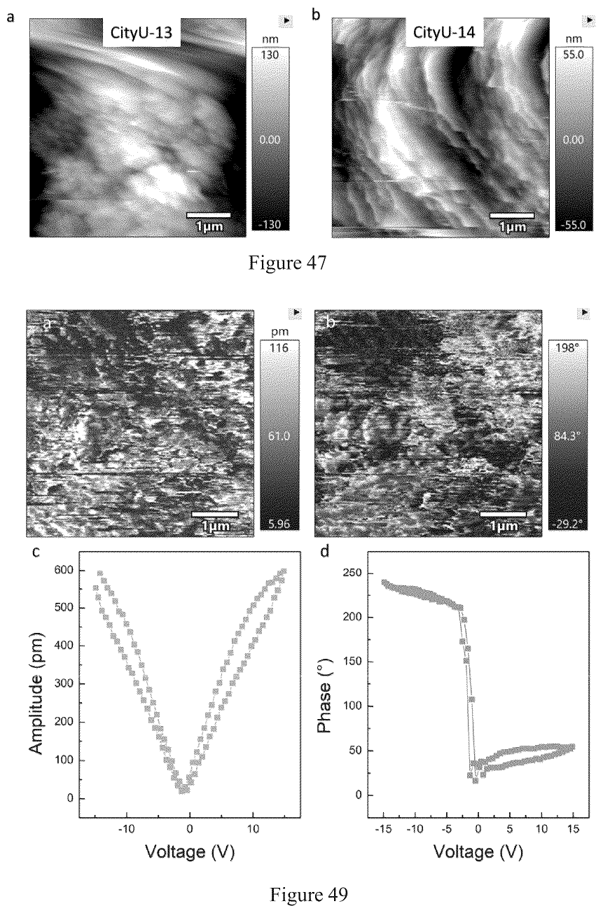

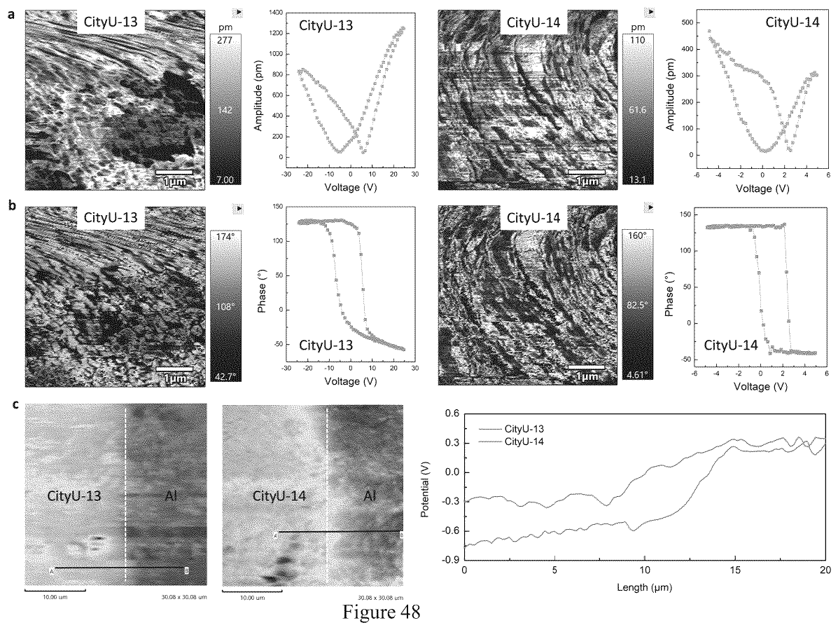

The parallel orientations of fluorinated chains in both 2D COFs gave rise to a polar structure with phonon confinement.^{[17]} The phonon confinement in both 2D COFs was demonstrated by location dependent Raman mapping (FIG. 46). By comparing Raman spectra at different positions, changes in phonon vibrational modes can be observed. In nanoscale crystalline materials, the non-uniformity of charge distribution (polarization) can be modulated by phonon confinement.^{[18]} Therefore, dipole alignment and net spontaneous polarization would be caused to induce piezoelectricity upon mechanical stress.^{[3a]} The piezoelectric responses of both COFs were performed using atomic force microscopy (AFM) with piezoelectric response force microscopy (PFM) and Kelvin probe force microscopy (KPFM).^{[3a, 5a, 19]} The topographic image of CityU-13 and CityU-14 acquired within the range of 5×5 μm² (FIG. 47). The vertical PFM amplitude and corresponding phase diagrams of both COFs were polled by the tip voltage of ±25 V from the same region. The dramatic differences in amplitude and corresponding phases with the change in bias indicate significant changes of intensity and polarization direction in the domain structures (FIGS. 48a, 48b).^{[20]} In order to verify the ferroelectric domains and switchable polarization in both COFs, nanoscale hysteresis loops underneath the PFM tip were further carried out. The butterfly shaped amplitude hysteresis loops and corresponding phase hysteresis loops dependent on bias voltage confirmed that the polarization of both samples could be switched by electric field.

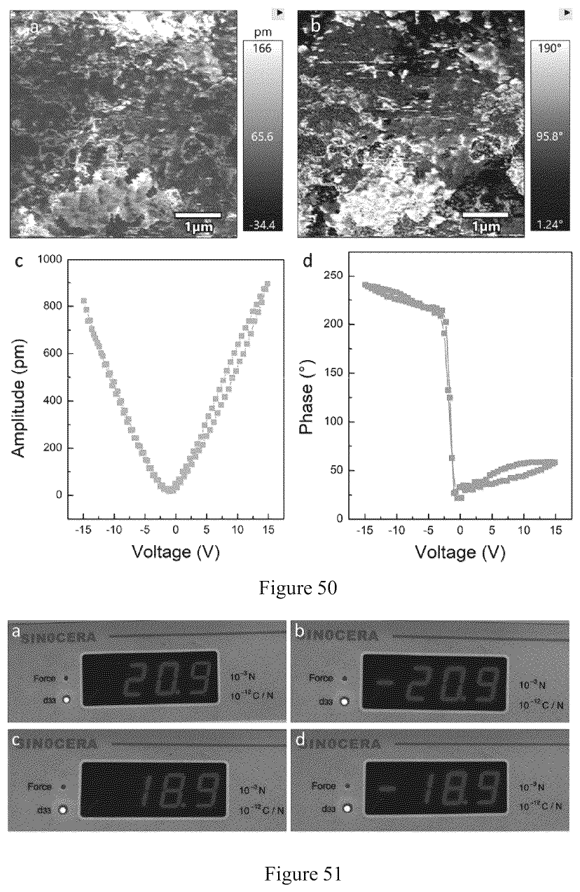

Moreover, lateral PFM amplitudes and phase images were collected. The corresponding amplitude butterfly loops and piezoresponse phase hysteresis loops further exhibited the change in polarization with the variation of bias voltages (FIGS. 49 and 50). Such a switchable nature of the domain structures provides robust evidence for the occurrence of piezoelectricity in CityU-13 and CityU-14.

TABLE S3
DFT simulation for d₃₃ coefficients of CityU-13.
Piezoelectric stress constants eᵢⱼ of CityU-13 is as follow:
$[ \begin{matrix}- 0.01382 & 0.03002 & - 0.0017 & 0.00905 & 0.02437 & 0.00745 \\ 0.01675 & 0.03327 & 0.00015 & 0.01134 & 0.02317 & 0.0077 \\ 0.01206 & - 0.00881 & - 0.00176 & - 0.00205 & - 0.00047 & - 0.01693\end{matrix} ]$

TABLE S4
DFT simulation for d₃₃ coefficients of CityU-14.
Piezoelectric stress constants eᵢⱼ of CityU-14 is as follow:
$[ \begin{matrix}0.00125 & 0.01315 & 0.00043 & 0.01357 & 0.01053 & 0.00432 \\ 0.01724 & 0.03052 & 0.00125 & 0.00983 & 0.01953 & 0.00929 \\ 0.00146 & - 0.01308 & - 0.00055 & - 0.00619 & - 0.00363 & - 0.01709\end{matrix} ]$
Elastic stiffness constants cᵢⱼ of CityU-14 is as follow:
$[ \begin{matrix}- 6.2 & 14.63 & 24.65 & 7.64 & - 6.2 & - 9.62 \\ 14.63 & 39.27 & 21.24 & - 4.02 & - 20.77 & - 5.35 \\ 24.65 & 21.24 & 59.65 & - 9.9 & 9.01 & - 16.47 \\ 7.64 & - 4.02 & - 9.9 & 13.27 & - 0.16 & - 24.21 \\ - 6.2 & - 20.77 & 9.01 & - 0.16 & 15.67 & 0.7 \\ - 9.62 & - 5.35 & - 16.47 & - 24.21 & 0.7 & - 20.75\end{matrix} ]$
Then piezoelectric strain constants matrix dᵢⱼ of CityU-14 is as follow:
$[ \begin{matrix}4.37 & - 13.8 & 5.25 & - 2.45 & - 18.23 & - 0.81 \\ 6.25 & - 24.43 & 10.07 & - 4.63 & - 33.22 & - 1.21 \\ 2.41 & - 8.54 & 3.23 & - 2.3 & - 11.75 & - 0.83\end{matrix} ]$

Furthermore, the direct piezoelectric effect of both COFs was determined by a quasi-static method. High piezoelectric coefficient d₃₃ (FIG. 51) was obtained for CityU-13 (20.9 pC/N) and CityU-14 (18.9 pC/N). In addition, the elastic stiffness tensor cᵢⱼ and piezoelectric stress coefficients eᵢⱼ of both COFs were calculated using density functional theory (DFT) to further investigate the piezoelectricity (Tables S3 and S4). The elastic stiffness tensor cᵢⱼ of CityU-13 is:

$$[ \begin{matrix}- 17.69 & 15.74 & 11.31 & - 3.93 & - 8.43 & 8.53 \\ 15.74 & 13.43 & - 22.07 & - 1.86 & - 18.45 & - 0.38 \\ 11.31 & - 22.07 & 12.25 & - 1.12 & 4.04 & 8.81 \\ - 3.93 & - 1.86 & - 1.12 & 9.47 & - 3.03 & - 2.86 \\ - 8.43 & - 18.45 & 4.04 & - 3.03 & 26.32 & 4.87 \\ 8.53 & - 0.38 & 8.81 & - 2.86 & 4.87 & - 27.29\end{matrix} ]$$

And the cᵢⱼ of CityU-14 is provided in Table S4. Subsequently, the piezoelectric strain constants matrix dᵢⱼ can be derived from the equation as follow:

$${d}_{ij} = {e}_{ik} \times {s}_{ij}$$

where sᵢⱼ denotes elastic compliance constants.^{[21]} The elastic compliance constants sᵢⱼ could be directly obtained by inverting the elastic stiffness matrix cᵢⱼ.

The di matrix of CityU-13 are as follows:

$$[ \begin{matrix}- 28.23 & - 33.48 & - 15.99 & - 32.31 & - 29. & - 15.86 \\ - 30.63 & - 36.16 & - 17.25 & - 34.72 & - 31.56 & - 17.2 \\ - 10.94 & - 12.96 & - 5.24 & - 13.18 & - 11.98 & - 6.92\end{matrix} ]$$

In the dᵢⱼ matrix, the piezoelectric strain constants d₃₃ of CityU-13 and CityU-14 were −5.24 pC/N and 3.2 pC/N, respectively. Therefore, the PFM results were consistent with the calculated dᵢⱼ matrix and experimental d₃₃ value, testifying the high piezoelectric response of both COFs.

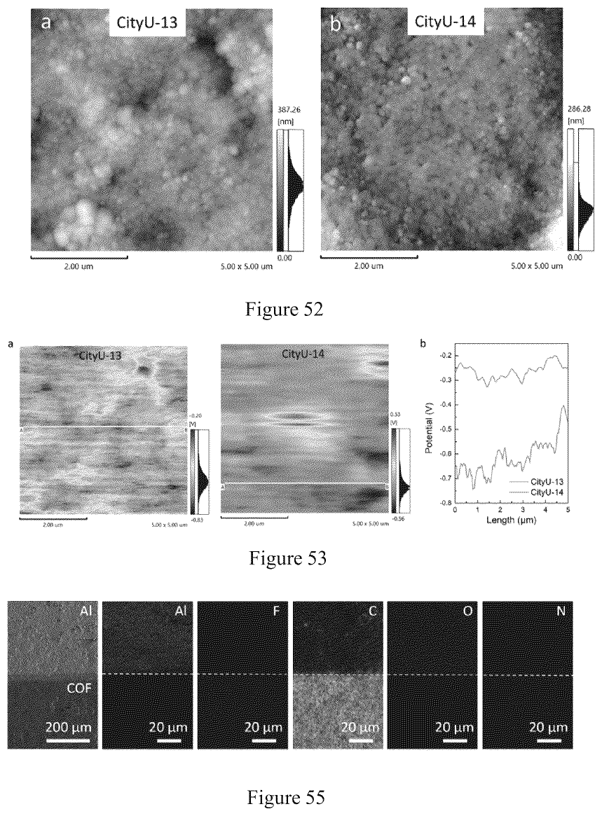

Furthermore, the Kelvin probe force microscope (KPFM) was employed to measure surface potential and provide powerful evidence for piezoelectric properties. The topological images showed uniform morphologies of both COFs (FIG. 52). Under the stress of the KPFM probe tip, an inward built-in electric field formed inside both COFs could migrate electrons outward to the surface of COFs. Therefore, a negative surface voltage of about 0.6 V for CityU-13 and 0.3 V for CityU-14 was generated (FIG. 53).^{[5a]} The negative surface potential was uniformed distributed on the surface of both COFs, which was more obvious compared to the aluminum (Al) substrate (FIG. 48c).

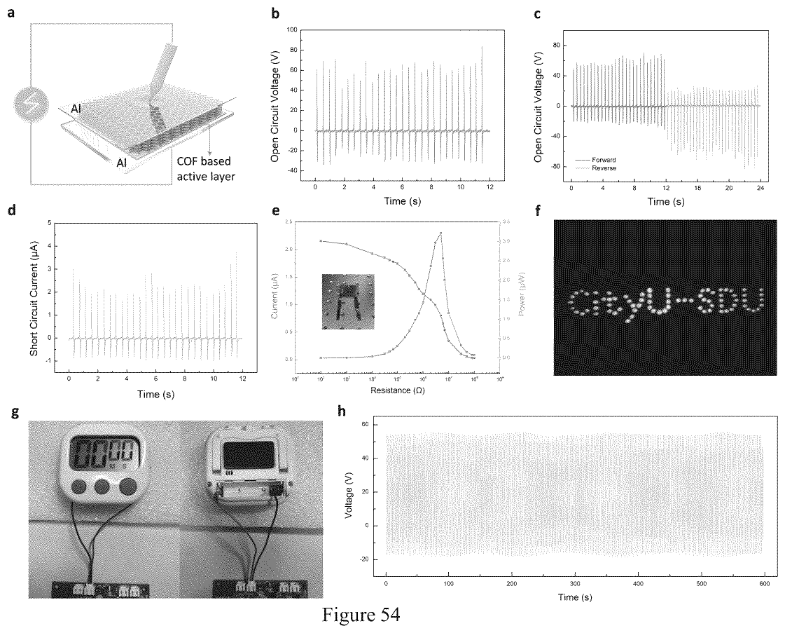

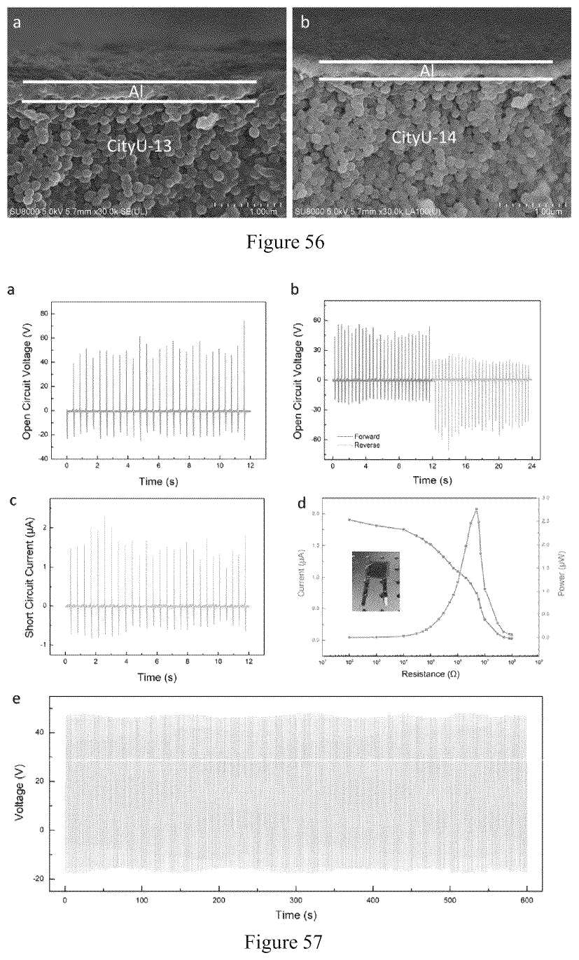

The exceptional piezoelectric properties of both COFs inspire us to further investigate their energy harvesting performance. PENGs with a sandwiched structure of Al/COF/Al were fabricated (FIG. 54a), which was confirmed through cross-sectional SEM image and corresponding elemental mappings (FIGS. 55 and 56). The superior piezoelectricity of both COFs brings about high open circuit voltage (V_{oc}) for PENG based on CityU-13 (60 V, FIG. 54b) or CityU-14 (50 V, FIG. 57a). The negative voltage was ascribed to the damping effect and inherent piezoelectric properties.^{[6]} The positive voltage originated from the dipole alignment and net spontaneous polarization upon mechanical contact, where dipoles came from substituted F-containing alkyl chains. The dipole alignment and net spontaneous polarization were further confirmed by the polarity switching test (FIG. 54c and FIG. 57b), where the voltage peaks could be positive for forward connection and negative for reverse connection. This result confirms that the origin of electrical output is only from the COFs, further evidencing the dipole alignment in both COFs.^{[22]}

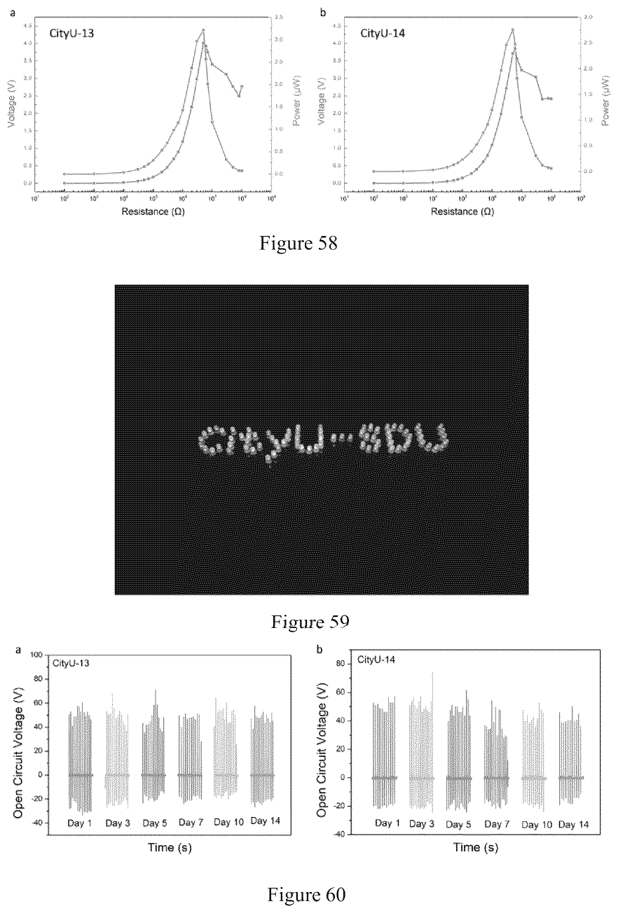

The short circuit current (I_{sc}) of PENGs based on CityU-13 and CityU-14 were 2.4 (FIG. 54d) and 1.5 μA (FIG. 57c). The output current gradually raised with the increase of external loading resistances (FIG. 54e and FIG. 57d). The currents of COFs-based PENGs were changed upon various resistance, indicating the current originated from both COFs rather than the input power source. Furthermore, the instantaneous power (P) of PENGs based on both COFs was calculated by varying the loading resistances from 100Ω to 100 MΩ. The instantaneous power of the CityU-13 based PENG device reached 3.3 μW at the 5 MΩ load resistance (FIG. 54e and FIG. 58a) while the CityU-14 based PENG device obtained 2.8 μW at the same load resistance (FIGS. 57d and 58b). The exceptional power output of both COFs demonstrates their proficiency in harvesting sustainable energy from mechanical impacts. Hence, they will be well-suited for self-powered electronic devices, eliminating the necessity for any external batteries. To verify the practical performance, CityU-13 based PENG was employed to directly power up an array of 76 light emitting diodes (LEDs) with the word pattern of “CityU-SDU” (FIG. 54f, FIG. 59) and a LED display timer (FIG. 54g) by using the full-wave bridge rectifier circuit and capacitor.

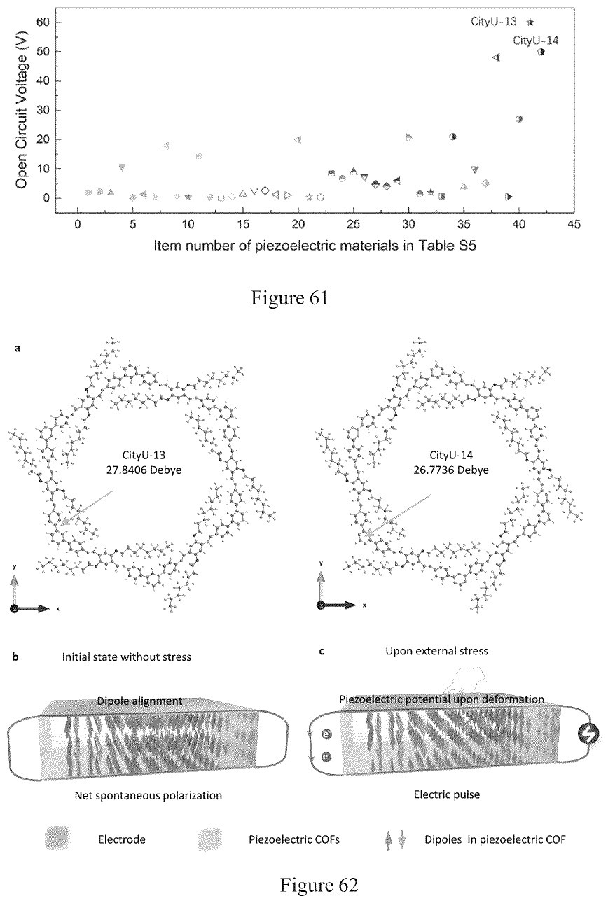

Crest factor (CF) and energy conversion efficiency (i) were used as the evaluation indicator of the output performance of PENG. According to the equations in previous reports,^{[4c, 23]} CF was obtained as 1.69 and 1.45 for PENG based on CityU-13 and CityU-14 separately. The η of PENG based on CityU-13 and CityU-14 were reached to 34.5% and 13.5%, respectively. Moreover, stability is another pivotal parameter for practical applications. Superior long-term durability of both COFs without any noticeable degradations of piezoelectric output voltage were further revealed under continuous force over a long period (600s: FIG. 54h and FIG. 57e; or even longer duration of 14 days: FIG. 60). The steady energy generation provides a good foundation for the device integration. The exceptional performance of both COFs-based PENGs, especially for the output open circuit voltage, is currently the highest among all reported organic piezoelectric materials (FIG. 61 and Table S5).

To get more insight into the mechanism of PENGs based on piezoelectric COFs, dipole moments and polarization were analyzed. The high electron-negativity of the F atom within the fluorinated side chains of both COFs results in robust polarized C—F bonds, analogous to those found in polyvinylidene fluoride (PVDF), ultimately leading to a significant dipole moment. The dipole moments of CityU-13 and CityU-14 were calculated to be 27.84 Debye and 26.77 Debye, respectively (FIG. 62a). Besides, both isoreticular COFs with ordered nanostructures could direct in-situ alignment of methylene (—CH₂) and difluoromethylene (—CF₂) moieties in fluorinated polar chains. The dipoles from those moieties in polar fluorinated alkyl chains can be aligned, leading to non-zero net spontaneous polarization (permanent polarization, FIG. 62b).^{[1b, 2-3]} When the mechanical stress is applied on the COF-based PENG devices, the opposite electrical charges will be spatially separated to generate piezoelectric potentials due to the dipoles (FIG. 62c).^{[24]} The free electrons present within the circuit traverse the external load, compensating for the field established by the dipoles. This compensation gives rise to an electric pulse as a reaction to the mechanical deformation. Furthermore, when the external pressure exerted on the PENG device is lifted, the piezoelectric potential existing between the electrodes dissipates. Consequently, the electrons that had accumulated at the upper electrode revert back to the lower side through the circuit, thereby generating an electric pulse in the converse direction.^{[1a]}

TABLE S5
Comparison of piezoelectric performances with other organic piezoelectric materials.

| # | Piezoelectric materials | V_{oc} | | I_{sc} | | d₃₃ | | Reference |
|---|---|---|---|---|---|---|---|---|
| 1 | Acceptor-donor-acceptor | 2.0 | V | 29.15 | nA/cm² | 36 | pm/V | Chem. Mater. 2023, |
| | (ADA)-type ambipolar | | | | | | | 35, 6463-6471 |
| | π-systems: AD₁A | | | | | | | |
| 2 | Acceptor-donor-acceptor | 2.2 | V | 45.64 | nA/cm² | 68 | pm/V | Chem. Mater. 2023, |
| | (ADA)-type ambipolar | | | | | | | 35, 6463-6471 |
| | π-systems: AD₂A | | | | | | | |
| 3 | Acceptor-donor-acceptor | 1.9 | V | 27.00 | nA/cm² | 26 | pm/V | Chem. Mater. 2023, |
| | (ADA)-type ambipolar | | | | | | | 35, 6463-6471 |
| | π-systems: AD₃A | | | | | | | |
| 4 | TMAB | 10.85 | V | 0.42 | μA | 10-16 | pC/N | Angew. Chem. Int. |
| | | | | | | | | Ed. 2020, 59, 7808- |
| | | | | | | | | 7812 |
| 5 | BTA-C₆-NDI₃ | 0.25 | V | 0.4 | μA | 5 | pm/V | Chem. Mater. 2023, |
| | | | | | | | | 35, 3316-3328 |
| 6 | Pro-Phe-Phe assemblies | 1.4 | V | 52 | nA | 2.2 | pm/V | Nat. Commun. 2021, |
| | | | | | | | | 12, 2634 |
| 7 | Hyp-Phe-Phe assemblies | 0.45 | V | 39.3 | nA | 4 | pm/V | Nat. Commun. 2021, |
| | | | | | | | | 12, 2634 |
| 8 | HFPD | 18 | V | / | | ~138 | pC/N | Science 2024, 383, |
| | | | | | | | | 1492-1498 |
| 9 | Aliphatic amino acid | 0.84 | V | 35 | nA | 11.4 | pm/V | ACS Appl. Mater. |
| | arrays | | | | | | | Interfaces 2022, 14, |
| | | | | | | | | 46304-46312 |
| 10 | γ-glycine | 0.45 | V | / | | 9.93 | pm/V | Nat. |
| | | | | | | | | Mater. 2018, 17, 180- |
| | | | | | | | | 186 |
| 11 | β-glycine nanocrystalline | 14.5 | V | 4 | μA | 11 | pm/V | Nat. Commun. 2023, |
| | films | | | | | | | 14, 4094 |
| 12 | L-Tyr crystal film | 0.5 | V | 35 | nA | / | | ACS Nano 2019, 13, |
| | | | | | | | | 12, 14477-14485 |
| 13 | DL-alanine network | ~150 | mV | 0.587 ± | | 10.34 | pm/V | Nat. Commun. 2023, |
| | | | | 0.118 nA | | | | 14, 6562 |
| 14 | 4,4′-Bpy crystal film | 0.65 | V | / | | / | | ACS Nano 2020, 14, |
| | | | | | | | | 8, 10704-10715 |
| 15 | FF-microrod | 1.4 | V | 39.2 | nA | 17.9 | pm/V | Nat. Commun. 2016, |
| | | | | | | | | 7, 13566 |
| 16 | FF-nanotube | 2.8 | V | 37.4 | nA | / | | ACS Nano 2018, 12, |
| | | | | | | | | 8, 8138-8144 |
| 17 | FF/porphyrin film | 2.6 | V | 5.8 | μA/cm2 | 62.3 | pm/V | |
| 18 | Cyclo-GW | 1.2 | V | 1.75 | nA | / | | Adv. Mater. 2019, |
| | | | | | | | | 31, 1807481 |
| 19 | Boc-Dip-Dip | 1 | V | 60 | nA | 73 | pC/N | ACS Nano 2020, 14, |
| | | | | | | | | 6, 7025-7037 |
| 20 | [Bn(4-BrBn)NMe₂]•BF₄ | 20 | V | 4 | μA | 2.16 | pC/N | Chem. - Asian |
| | | | | | | | | J. 2021, 16, 4122- |
| | | | | | | | | 4129 |
| 21 | BPA/Ac-D-Ala | 0.32 | V | / | | / | | J. Am. Chem. Soc. |
| | | | | | | | | 2022, 144, |
| | | | | | | | | 18375-18386. |
| 22 | BPA/Ac-L-Ala | 0.35 | V | / | | / | | J. Am. Chem. Soc. |
| | | | | | | | | 2022, 144, |
| | | | | | | | | 18375-18386. |
| 23 | DPDP•PF₆/PDMS | 8.5 | V | 0.28 | μA cm⁻² | 8 | pC/N | Angew. Chem., Int. |
| | | | | | | | | Ed. 2018, 57, 9054- |
| | | | | | | | | 9058 |
| 24 | DPDP•PF₆/TPU | 6.73 | V | 0.47 | μA | / | | Chem. Mater. 2019, |
| | | | | | | | | 31, 15, 5964-5972 |
| 25 | DPDP•BF₄/TPU | 8.95 | V | 0.89 | μA | 7 | pC/N | Chem. Mater. 2019, |
| | | | | | | | | 31, 15, 5964-5972 |
| 26 | TPAP•BF₄/TPU | 7.37 | V | 0.61 | μA | 3 | pC/N | Chem. Mater. 2019, |
| | | | | | | | | 31, 15, 5964-5972 |
| 27 | TIAP•BF₄/TPU | 4.75 | V | 0.41 | μA | 3 | pC/N | Chem. Mater. 2019, |
| | | | | | | | | 31, 15, 5964-5972 |
| 28 | glycine-PVA films | 4.1 | V | 360 | nA | 5 to 6 | | Science 2021, 373, |
| | | | | | | pC/N | | 337-342 |
| 29 | Nylon-11 fiber | 6 | V | 3.8 | μA | / | | Adv. Funct. Mater. |
| | | | | | | | | 2021, 31, 2004326 |
| 30 | PVDF nanofiber | 20.8 | V | 0.06 | μA | 5 | pC/N | ACS Appl. Electron. |
| | | | | | | | | Mater. 2021, 3, 6, |
| | | | | | | | | 2738-2747 |
| 31 | PVDF-TrFE (0.73:0.27) | 1.5 | V | 40 | nA | / | | Nat. Commun. 2013, |
| | | | | | | | | 4, 1633. |
| 32 | PVDF-TrFE | ~2 | V | ~0.5 | μA | 37 | pm/V | Adv. Sci. 2023, 10, |
| | | | | | | | | 2205942 |
| 33 | Electrospun P(VDF- | 700 | mV | 170 | nA | 56 | pm/V | J. Mater. Chem. |
| | TrFE) nanofibers | | | | | | | A, 2016, 4, 2293- |
| | | | | | | | | 2304 |
| 34 | Porous electrospun | 21.0 ± | | 21.1 ± | | / | | Nano Energy 2019, |
| | P(VDF-TrFE) nanofibers | 1.2 V | | 1.2 μA | | | | 62, 594-600 |
| 35 | PVDF₇₉PU₂₁ nanofibers | 3.8 | V | 0.65 | μA | 7.64 | pm/V | ACS Appl. Polym. |
| | | | | | | | | Mater. 2022, 4, |
| | | | | | | | | 4751-4764 |
| 36 | PVDF-PANI nanofibers | 10 | V | 4 | μA/cm² | 32 | pC/N | ACS Appl. Polym. |
| | | | | | | | | Mater. 2020, 2, |
| | | | | | | | | 862-878 |
| 37 | 2FNC/PVDF | 5.11 | V | / | | / | | ACS Appl. Polym. |
| | | | | | | | | Mater. 2020, 2, |
| | | | | | | | | 2550-2562 |
| 38 | Ionic liquid based PVDF | 48 | V | 1.4 | μA | 17 | pC/N | ACS Appl. Electron. |
| | nanofibers P-IL-5 | | | | | | | Mater. 2021, 3, |
| | | | | | | | | 2738-2747 |
| 39 | 5 wt % UiO-66/PVDF | 0.536 | V | / | | / | | ACS Appl. Nano |
| | | | | | | | | Mater. 2020, 3, |
| | | | | | | | | 8742-8752 |
| 40 | 5 wt % MOF-5/PDMS | 27 | V | 2.9 | μA | / | | Nano Energy 2022, |
| | | | | | | | | 96, 107128 |
| 41 | Two-dimensional imine- | 60 | V | 2.4 | μA | 20.9 | pC/N | This work |
| | linked COF: CityU-13 | | | | | | | |
| 42 | Two-dimensional imine- | 50 | V | 1.5 | μA | 18.9 | pC/N | This work |

linked COF: CityU-14

### Materials

All reagents and solvents were purchased and used directly without further purification. Cesium carbonate (Cs₂CO₃), sodium hydroxide (NaOH) and p-Toluenesulfonyl Chloride (TsCl) were purchased from Macklin Ltd (Shanghai, China). 3,3,4,4,5,5,6,6,7,7,8,8,8-Tridecafluorooctan-1-ol (13F—OH) was bought from TCI Development Co. Ltd (Shanghai, China). 2,5-Dihydroxyterephthalaldehyde (DHTP), 1,3,5-tris(4-aminophenyl)benzene (TAPB) and tris(4-aminophenyl)triazine (TAPT) were obtained from Leyan Ltd (Shanghai, China). Acetic acid glacial (HOAc, >99%) was obtained from Sigma-Aldrich Chemical Ltd. (Shanghai, China). N,N-Dimethylformamide (DMF), tetrahydrofuran (THF), acetone, dichloromethane (DCM) and n-hexane were obtained from Anaqua Global International Inc Limited. Superdry 1,2-dichlorobenzene (DCB), n-butyl alcohol (n-BuOH) and acetonitrile (CH₃CN) were purchased from J&K chemical Ltd (Shanghai, China). Chloroform-d(CDCl₃, 99.8 atom % D) was obtained from CIL (Cambridge Isotope Laboratories Inc). Polydimethylsiloxane (PDMS) was obtained from Dow chemical company. Deionized (DI) water was obtained using a Stakpure OmniaLab40ED+ ultrapure water system (18.2 MΩ/cm). The synthetic routes of precursors 3,3,4,4,5,5,6,6,7,7,8,8,8-tridecafluorooctyl 4-methylbenzenesulfonate (13F-OTs) and 2,5-bis((3,3,4,4,5,5,6,6,7,7,8,8,8-tridecafluorooctyl)oxy) terephthalaldehyde (13F—CHO) were shown in Scheme S1 and S2 according to the previous reports with minor modifications.

### Instrumentation and Characterization

Fourier transform infrared (FTIR) spectra were recorded from 400 to 4000 cm⁻¹ on a PerkinElmer Spectrum Two spectrometer. ¹H NMR, ¹⁹F NMR and ¹³C NMR spectra were collected using a Bruker 400 MHz “AVANCE III” Nuclear Magnetic Resonance (NMR) spectrometer, where tetramethylsilane (TMS) was used as the internal standard for chemical shift. Powder X-ray diffraction (PXRD) patterns were recorded on a Rigaku X-ray Diffractometer (SmartLab™ 9 kW) with Cu Kα monochromatic radiation (λ=1.5406 Å) at 30 kV and 10 mA with scan rate of 0.1 degree/s. Ultralow-dose high-resolution transmission electron microscopy (HRTEM) images were conducted on a Cs-corrected FEI Titan Cube transmission electron microscope at an acceleration voltage of 300 kV. Prior to the TEM test, the suspension of two COFs in ethanol was ultrasonicated and dropped onto the carbon support copper grids for drying naturally in air. SEM images and elemental mappings of two COFs were taken on a Quattro S scanning electron microscope (Thermo Fisher), while the cross-section information and corresponding energy-dispersive spectroscopy (EDS) mapping of the active layers based on COFs with aluminum electrodes were observed through field emission scanning electron microscope (FEI-SEM, Helios G4 UC). Elemental information was further captured on a Thermo Fisher ESCALAB XI⁺ X-ray photoelectron spectrometer (XPS) with a monochromatic Al Kα radiation source (1486.6 eV), where the binding energy of C1s signal at 284.6 eV was used for the correction of different elements. Thermogravimetric analysis (TGA) curves were measured using a PerkinElmer Simultaneous Thermal Analyzer (STA) 6000 under N2 atmosphere (heating rate: 10° C./min). Prior to testing the nitrogen (N₂) sorption isotherms, two COFs were degassed at 120° C. overnight. The N₂ sorption isotherms for two COFs were tested on a Micromeritics 3Flex 3500 Multi-port High Throughput Gas Adsorption Analyzer at 77 K. The surface areas were evaluated using the Brunauer-Emmett-Teller (BET) approach and the pore size distributions were derived from the N₂ adsorption isotherm using the nonlocal density functional theory (NLDFT) method. The water contact angle measurements were performed on a Dataphysics OCA 15EC Contact Angle measuring device with SCA 20 software to calculate surface and interfacial tension from the contours of pendant and sessile drops. The piezoelectric coefficient d₃₃ was determined by the quasi-static d₃₃ meter (Zj-2, Institute of acoustics, Chinese Academy of Sciences).

### Synthesis of 13F-OTs

13F-OTs was synthesized according to the previous report with slight modification.[31] To be specific, 560 mg (13.76 mmol) of NaOH was loaded in the round bottom flask (RBF) before adding 16 mL of DI water and 16 mL of THF. After cooling down in the ice bath, 2.072 mL (9.6 mmol) of 13F—OH was injected to form solution 1.

Solution 2 was prepared by dissolving 1.664 g (8.96 mmol) of TsCl in 16 mL of THF. Then, solution 2 was dropwise added into solution 1. The resulted mixture was stirred for 8 hours before pouring into ice water. The white powder was harvested in 70% yield after filtration and dried under vacuum. ¹H NMR (400 MHz, CDCl₃) δ 7.80 (d, J=8.3 Hz, 2H), 7.37 (d, J=8.1 Hz, 2H), 4.30 (t, J=6.7 Hz, 2H), 2.52 (ddd, J=18.2, 12.4, 6.8 Hz, 2H), 2.46 (s, 3H). ¹⁹F NMR (376 MHz, CDCl₃) δ −80.87 (tt, J=10.1, 2.2 Hz), −113.46-−113.75 (m), −121.96 (d, J=11.4 Hz), −122.95 (dd, J=9.5, 3.7 Hz), −123.64 (d, J=13.5 Hz), −126.23 (ttd, J=10.4, 7.0, 3.4 Hz).

### Synthesis of 13F—CHO

13F—CHO was synthesized according to the previous literature with slight modification. [32] To be specific, 14 mg (0.084 mmol) of DHTP, 255 mg (0.492 mmol) of 13F-OTs and 243 mg of Cs₂CO₃ (0.748 mmol) were loaded into the RBF before three cycles of vacuum-N₂ purge. After injecting 10 mL of superdry CH₃CN into the RBF, the mixture was refluxed for 12 hours before extracting with dichloromethane (DCM), brine and DI water subsequently. The organic layer was collected, combined and evaporated. The resulting mixture was purified by column chromatography using the mixed solvent of DCM and n-hexane (1:2 in v/v) as eluent to afford pale yellow 13F—CHO. ¹H NMR (300 MHz, CDCl₃) δ 10.48 (s, 1H), 7.48 (s, 1H), 4.44 (t, J=6.2 Hz, 2H), 2.81-2.60 (m, 2H). 19F NMR (376 MHz, CDCl₃) δ −80.73 (t, J=9.9 Hz), −121.80 (s), −122.83 (s), −123.46 (s), −126.11 (td, J=14.8, 6.8 Hz).

### Synthesis of CityU-13

The synthesis of CityU-13 was similar to the previous report with minor modification.[33]. The details are shown below:

In a pyrex tube, 16 mg of 13F—CHO (0.018 mmol) and 4.5 mg of TAPB (0.012 mmol) were loaded. Then, 0.25 mL of superdry DCB and 0.25 mL of superdry n-BuOH were injected as the solvent and 50 μL of aqueous acetic acid solution (HOAc, 6M) was used as the catalyst. The resulted mixture was ultrasonicated for 15 minutes and degassed through three freeze-pump-thaw cycles before sealing under vacuum. Then, the sealed pyrex tube was put into the oven (120° C.) for 72 hours before cooled down to room temperature. The obtained yellow product was filtered and washed by DMF, THF, acetone and n-hexane subsequently. CityU-13 was obtained in 68% yield after Soxhlet extraction in THF overnight.

### Synthesis of CityU-14

The synthesis procedure of CityU-14 was similar to that of CityU-13 by replacing 4.5 mg of TAPB with 4.6 mg of TAPT. The washing process and Soxhlet extraction were also similar to that of CityU-13. Yield: 63%.

### Piezoresponse Force Microscopy (PFM) and Kelvin Probe Force Microscopy (KPFM) Measurements

The measurement of piezoresponse was carried out on Oxford PFM instrument (MFP-3D) at room temperature. Conductive Pt/Ir-coated silicon probes (EFM-10, Nanoworld) were used for domain imaging and polarization switching studies, with a force constant k of ˜2.8 N/m and a free-air resonance frequency of ˜75 kHz. We conducted the PFM experiments at contact resonance. KPFM measurement was performed in SPM9700HT atomic force microscope (Shimadzu). The interleave scan of Kelvin probe force microscopy (KPFM) was about 10 nm higher than the sample surface and the topographic scan was performed using a tapping mode. The spatial resolution of KPFM was in the order of several tens of nanometers. To minimize the tip effect, all the tests were conducted on the same tip for a specific sample. [34]

### Preparation of Active Layer and Fabrication of Piezoelectric Nanogenerator (PENG)

CityU-13 based active layer: The PDMS solution was prepared by adding a curing agent to the base at a weight ratio of 1:10. Then, CityU-13 (15 wt %) was blended into the PDMS solution. Followed by removing bubbles in a vacuum oven and spreading onto a glass substrate as piezoelectric film, the active layer was obtained after curing in an oven (80° C.) for 10 hours. [35]

CityU-14 based active layer: The preparation of CityU-14 based active layer was similar to that of CityU-13 based active layer except using CityU-14 (15 wt %) as active layers.

Piezoelectric nanogenerator (PENG): The as-fabricated active layer based on CityU-13 or CityU-14 was used as the intermediate part of a PENG device. Al metal electrodes with a thickness of ˜100 nm were evaporated on the COF film. The Cu tapes are attached onto Al metal electrodes using silver (Ag) paste for the characterization of the output voltage and current signals. All the devices were encapsulated with PET tape before testing. The output voltage and current of the as-fabricated PENGs were measured by applying a normal force in a linear motor. Piezoelectric output performances were characterized by the digital oscilloscope and the source meter (Keithley 6514).

### Structure Determination

Materials Studio (MS) 2020 suite of programs (BIOVIA) was employed to simulate the intrinsic connectivity and geometry of CityU-13 and CityU-14. [36] The initial structures of CityU-13 and CityU-14 were geometrically refined with the Forcite molecular dynamics module (Universal force fields, Ewald summations) to afford optimized arrangement with an AA-stacking pattern. [37] Then, Pawley refinement was performed in MS 2020 using the implemented Reflex package. The space groups of final unit cells of both COFs belong to P1 after the convergence of R_{wp} and Rₚ (See Table S4 and S5).

### DFT Simulations of Dipole Moment and Piezoelectric Coefficient

The geometry optimization and dipole moment were calculated at B3LYP/6-31G(d) level with Gaussian 16 software. Density functional theory (DFT) calculations were carried out using a Vienna ab initio simulation package (VASP). [38] The projected augmented wave (PAW) method is used to describe the electron-ion interactions. The exchange-correlation interaction is treated by the generalized gradient approximation (GGA) [39] in the form of Perdew-Burke-Ernzerh (PBE) functional. The cutoff energy was set to 550 eV. A k-grid of 2×2×3 was employed in electronic structure calculations. The DFT-D3 method was adopted to correct the van der Waals interaction. The convergence criteria for energy and force were set to 1×10⁻⁵ eV and 0.01 eV/A, respectively. The elastic stiffness was calculated with a finite difference method. [40] The piezoelectric constants were calculated using density functional perturbation theory (DFPT), [41] where the elastic stiffness constants of COFs were presented as a 6×6 matrix:

$$[ \begin{matrix}{c}_{11} & {c}_{12} & {c}_{13} & {c}_{14} & {c}_{15} & {c}_{16} \\ {c}_{21} & {c}_{22} & {c}_{23} & {c}_{24} & {c}_{25} & {c}_{26} \\ {c}_{31} & {c}_{32} & {c}_{33} & {c}_{34} & {c}_{35} & {c}_{36} \\ {c}_{41} & {c}_{42} & {c}_{43} & {c}_{44} & {c}_{45} & {c}_{46} \\ {c}_{51} & {c}_{52} & {c}_{53} & {c}_{54} & {c}_{55} & {c}_{56} \\ {c}_{61} & {c}_{62} & {c}_{63} & {c}_{64} & {c}_{65} & {c}_{66}\end{matrix} ]$$

The piezoelectric strain constants of COFs can be described by a third rank tensor in the form of a 3×6 matrix:

$$[ \begin{matrix}{d}_{11} & {d}_{12} & {d}_{13} & {d}_{14} & {d}_{15} & {d}_{16} \\ {d}_{21} & {d}_{22} & {d}_{23} & {d}_{24} & {d}_{25} & {d}_{26} \\ {d}_{31} & {d}_{32} & {d}_{33} & {d}_{34} & {d}_{35} & {d}_{36}\end{matrix} ]$$

The piezoelectric strain constants matrix dᵢⱼ can be derived from the following equation:

$${d}_{ij} = {e}_{ik} \times {s}_{ij}$$

where eᵢₖ and sᵢⱼ denote piezoelectric stress constants and elastic compliance constants that obtained from theoretical calculations. sᵢⱼ equal to the inverse matrix of cᵢⱼ. By inverting the elastic stiffness matrix cᵢⱼ, we can directly obtain the elastic compliance.

### PENG Output Measurement

The value of the instantaneous power of each PENG was obtained according to the equation as follow,

$$P = {I}^{2} R$$

where I illustrates the measured current while R represents the load resistance.

Crest factor is used as the evaluation indicator of the output performance of PENG. According to the previous reports, crest factor (CF) can be determined using the root-mean-square value (I^{RMS}) and extreme difference value (I_{EDV}) with the following equation.

$${I}_{RMS} = \sqrt{\frac{{\int }_{0}^{t} {I}_{SC}^{2} dt}{t}} = \sqrt{\frac{{\sum }_{i = 1}^{n} {I}_{SCi}^{2}}{n}}$$

$${I}_{ED} = {I}_{MAX} - {I}_{MI}$$

$$CF = \frac{{I}_{MAX}}{{I}_{RMS}}$$

For CityU-13 based PENG, IMAX was determined as 4.17 μA, I_{RMS} was determined as 2.46, therefore CF was obtained as 1.69. For CityU-14 based PENG, IMAX and I_{RMS} were 2.33 μA and 1.61, which produced a smaller CF of 1.45.

The energy conversion efficiency (η) is defined as the ratio of output electrical to input kinetic energy. In this case, the output energy is the electrical energy from PENG, while the input energy is the kinetic energy (KE) applied to PENG. KE is defined as: [43]

$${E}_{k} = \frac{1}{2} {mv}^{2}$$

m is load mass and v is moving velocity of load. The average load mass (m) was calculated to be 0.05 kg. The velocity (v) was calculated as 0.2 m/s using v=x/t, where x=20 cm is the moving distance of the load mass and t is 1 s. So, kinetic energy (KE) can be calculated as 0.001 J. The output electrical energy (E_{electrical}) of both COF based PENG was measured on the load resistance of 5 MΩ. The E_{electrical} can be calculated as follows: [43]

$${E}_{electrical} = {\int }_{0}^{t} {I}^{2} Rdt$$

I is the measured current, R is load resistance, and t is measuring time. The output electric energy from PENG of CityU-13 and CityU-14 were obtained as 0.000345 J and 0.000135 J. The energy conversion efficiency (η) was calculated as

$$Energy Conversion Efficiency ( η ) = \frac{output electric energy}{input kinetic energy} \times 100 \%$$

Consequently, the η of PENG based on CityU-13 and CityU-14 were reached to 34.5% and 13.5%, respectively.

In summary, one can see from the above that fluorinated alkyl chains-functionalized 2D COFs (CityU-13 and CityU-14) can introduce asymmetry, polarity and net spontaneous polarization for piezoelectricity. Benefiting from the collective contribution of high crystallinity and ordered arrangement for dipole alignment, both COFs displayed excellent piezoelectricity. Moreover, PENGs based on both COFs displayed high d₃₃, impressive output open circuit voltage, short circuit current, instantaneous power and excellent stability. We also demonstrated that the PENGs can successfully power up an array of 76 LEDs and the LED display timer, confirming the practical application of piezoelectric COFs. Our results would provide an alternative strategy to design new piezoelectric materials for self-powering systems.

The exemplary embodiments are thus fully described. Although the description referred to particular embodiments, it will be clear to one skilled in the art that the invention may be practiced with variation of these specific details. Hence this invention should not be construed as limited to the embodiments set forth herein.

While the embodiments have been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

What is claimed is:

1. A two-dimensional covalent organic framework (COF) compound, comprising piezoelectric monomers which are inter-connected via covalent bonds; each of the piezoelectric monomers comprising fluorinated alkyl chains tethered on peripheral positions of an aromatic moiety.

2. The COF compound of claim 1, wherein the fluorinated alkyl chains tethered on the aromatic moiety comprise asymmetric fluorinated alkyl chains.

3. The COF compound of claim 1, wherein one of the fluorinated alkyl chains comprises at least one methylene group that is substituted by fluorine.

4. The COF compound of claim 1, wherein at least some of the fluorinated alkyl chains have different lengths.

5. The COF compound of claim 1, wherein one of the fluorinated alkyl chains comprises at least one carbon.

6. The COF compound of claim 5, wherein the one of the fluorinated alkyl chains is 3,3,4,4,5,5,6,6,7,7,8,8,8-trideca-fluorooctyl side chain.

7. The COF compound of claim 1, wherein at least one of the fluorinated alkyl chains comprises one or more difluoromethylene (—CF₂) moieties.

8. The COF compound of claim 7, wherein each one of the fluorinated alkyl chains comprises one or more difluoromethylene (—CF₂) moieties.

9. The COF compound of claim 1, wherein the piezoelectric monomers comprise two or more different monomers connected to each other.

10. The COF compound of claim 9, wherein the two or more different monomers comprise triamino-/tetraamino-monomers in condensation reaction with dialdehyde-monomers, or diamino-monomers in condensation reaction with trialdehyde-/tetraaldehyde-monomers.

11. A method of synthesizing the COF compound of claim 1, comprising steps of:
a) providing an aromatic precursor; and
b) anchoring fluorinated alkyl chains onto the aromatic precursor.

12. The method of claim 11, wherein the aromatic precursor comprises hydroxyl groups or halogen atoms on the peripheral positions, which facilitate fluorinated reaction.

13. The method of claim 11, wherein Step b) further comprises:
c) mixing the aromatic precursor with a fluorinated agent and a solvent to obtain a reaction mixture.

14. The method of claim 13, wherein the solvent is chosen from a group consisting of N,N-Dimethylformamide, 1,2-dichlorobenzene, dichloromethane, 1,2-dichloroethane, tetrahydrofuran, n-hexane, ethyl acetate, toluene, acetonitrile, or a mixture thereof.

15. The method of claim 13, wherein Step c) is conducted in an alkaline condition.

16. The method of claim 13, further comprising steps of:
d) quenching the reaction mixture;
e) filtering the reaction mixture;
f) washing the reaction mixture; and
g) drying the reaction mixture.

17. A piezoelectric nanogenerator device, comprising:
a) a top electrode;
b) a bottom electrode; and
c) an active layer configured between the top electrode and the bottom electrode; the active layer being fabricated using the COF compound of claim 1.

18. A method of preparing an active layer for a piezoelectric nanogenerator device, comprising the steps of:
a) mixing a COF compound of claim 1 with a polymer material and a solvent to form a mixture; and
b) drying the mixture to obtain the active layer.
